(19) United States
(12) Patent Application Publication
(10) Pub. No.: US 2026/0131026 A1
(43) Pub. Date: May 14, 2026

(54) GENE THERAPY FOR OCULAR DISEASE

(71) Applicant: Opus Genetics Inc., Durham, NC (US)

(72) Inventors: Mayur Choudhary, Morrisville, NC (US); Ashwath Jayagopal, Durham, NC (US); Benjamin Yerxa, Raleigh, NC (US)

(21) Appl. No.: 19/259,625

(22) Filed: Jul. 3, 2025

Related U.S. Application Data
(63) Continuation of application No. PCT/US2024/010335, filed on Jan. 4, 2024.
(60) Provisional application No. 63/437,346, filed on Jan. 5, 2023.
(60) Provisional application No. 63/497,850, filed on Apr. 24, 2023.

(51) Int. Cl. A61K 48/00; A61K 38/45; A61P 27/02; C12N 9/12; C12N 15/86

(52) U.S. Cl. CPC A61K 48/005; A61K 38/45; A61K 48/0075; A61P 27/02; C12N 9/12; C12N 15/86; C12Y 207/10001; C12N 2750/14143

(57) ABSTRACT

Methods and compositions for gene therapy of retinal degeneration related to mutations in MER proto-oncogene, tyrosine kinase (MERTK).

### CROSS-REFERENCE

This Application is a Continuation of Application PCT/US2024/010335 filed on Jan. 4, 2024. Application PCT/US2024/010335 claims the benefit of U.S. Provisional Application 63/437,346 filed on Jan. 5, 2023, and U.S. Provisional Application 63/497,850 filed on Apr. 24, 2023. The entire contents of these applications are incorporated herein by reference in their entirety.

### SUBMISSION OF SEQUENCE LISTING XML

The instant application contains a Sequence Listing which has been submitted electronically in XML format and is hereby incorporated by reference in its entirety. Said XML file, created on Jul. 3, 2025, is named “219504_seqlist.xml” and is 58,749 bytes in size.

### BACKGROUND

MER proto-oncogene, tyrosine kinase (MERTK) is a transmembrane protein that is a part of the MER/AXL/TYRO3 receptor kinase family. MERTK is involved in vision, and mutations in the MERTK gene are implicated in multiple ocular diseases.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Provided herein are methods of treating a subject with an eye disease or disorder, the methods comprising administering to the subject a nucleic acid comprising a nucleotide sequence at least 90% identical to any one of SEQ ID NOs: 3-6. In some embodiments, the nucleic acid comprises a nucleotide sequence at least 90% identical to SEQ ID NO: 3. In some embodiments, the nucleic acid comprises a nucleotide sequence at least 95% identical to SEQ ID NO: 3. In some embodiments, wherein the nucleic acid comprises a nucleotide sequence of SEQ ID NO: 3. In some embodiments, the nucleic acid comprises a nucleotide sequence at least 90% identical to SEQ ID NO: 4. In some embodiments, the nucleic acid comprises a nucleotide sequence at least 95% identical to SEQ ID NO: 4. In some embodiments, the nucleic acid comprises a nucleotide sequence of SEQ ID NO: 4. In some embodiments, the nucleic acid comprises a nucleotide sequence at least 90% identical to SEQ ID NO: 5. In some embodiments, the nucleic acid comprises a nucleotide sequence at least 95% identical to SEQ ID NO: 5. In some embodiments, the nucleic acid comprises a nucleotide sequence of SEQ ID NO: 5. In some embodiments, the nucleic acid comprises a nucleotide sequence at least 90% identical to SEQ ID NO: 6. In some embodiments, the nucleic acid comprises a nucleotide sequence at least 95% identical to SEQ ID NO: 6. In some embodiments, the nucleic acid comprises a nucleotide sequence of SEQ ID NO: 6. In some embodiments, the nucleotide sequence is comprised in a vector. In some embodiments, the vector is an adeno-associated viral (AAV) vector. In some embodiments, the AAV vector is a recombinant AAV (rAAV) vector. In some embodiments, the rAAV vector is selected from the group consisting of AAV2, AAV5, AAV8, AAV9, AAV2/5, AAV 2ŁYF, and AAV2.7m8. In some embodiments, the vector comprises an AAV capsid protein. In some embodiments, the AAV capsid protein is selected from the group consisting of an AAV2 capsid protein, an AAV2YF capsid protein, an AAV5 capsid protein, an AAV8 capsid protein, an AAV9 capsid protein, and a AAV2.7m8 capsid protein. In some embodiments, the vector further comprises: 1) a 5′ AAV ITR, and 2) a 3′ AAV ITR. In some embodiments, the vector comprises an AAV expression cassette. In some embodiments, the vector further comprises a rhodopsin (RHO) promoter. In some embodiments, the vector further comprises a CBA promoter. In some embodiments, the vector further comprises a VMD2 promoter. In some embodiments, the vector further comprises a human rhodopsin kinase (hGRK1) promoter. In some embodiments, the vector further comprises a CASI promoter. In some embodiments, the disease or disorder comprises a MERTK-mediated disease or disorder. In some embodiments, the MERTK-mediated disease comprises retinitis pigmentosa. In some embodiments, the disease or disorder comprises vision loss. In some embodiments, the administering occurs prior to onset of the disease or disorder. In some embodiments, the administering occurs after onset of the disease or disorder. In some embodiments, the administering occurs in at least one eye of the subject. In some embodiments, the administering is performed by subretinal injection, intravitreal injection, or suprachoroidal injection. In some embodiments, the administering is performed at an amount of at least 109 viral particles per mL. In some embodiments, the administering restores at least partial vision of the subject. In some embodiments, the administration improves vision loss by at least 10% when measured using a visual field test. In some embodiments, the subject is a mammal. In some embodiments, the subject is a human.

Also provided herein are functional MERTK nucleic acid sequences, or fragments thereof, comprising a nucleic acid sequence at least 90%, 95%, or 99% identical to any one of SEQ ID NOs: 3-6. Also provided herein are compositions comprising functional MERTK nucleic acid sequences, or fragments thereof. In some embodiments, the compositions further comprise a promoter which expresses a product of the functional MERTK nucleic acid sequence in a plurality of photoreceptor cells or retinal pigment epithelium cells. In some embodiments, the compositions further comprise a pharmaceutically acceptable carrier. In some embodiments, the nucleic acid sequence comprises a nucleotide sequence at least 90% identical to SEQ ID NO: 3. In some embodiments, the nucleic acid sequence comprises a nucleotide sequence at least 95% identical to SEQ ID NO: 3. In some embodiments, the nucleic acid sequence comprises a nucleotide sequence of SEQ ID NO: 3. In some embodiments, the nucleic acid sequence comprises a nucleotide sequence at least 90% identical to SEQ ID NO: 4. In some embodiments, the nucleic acid sequence comprises a nucleotide sequence at least 95% identical to SEQ ID NO: 4. In some embodiments, the nucleic acid sequence comprises a nucleotide sequence of SEQ ID NO: 4. In some embodiments, the nucleic acid sequence comprises a nucleotide sequence at least 90% identical to SEQ ID NO: 5. In some embodiments, the nucleic acid sequence comprises a nucleotide sequence at least 95% identical to SEQ ID NO: 5. In some embodiments, the nucleic acid sequence comprises a nucleotide sequence of SEQ ID NO: 5. In some embodiments, the nucleic acid sequence comprises a nucleotide sequence at least 90% identical to SEQ ID NO: 6. In some embodiments, the nucleic acid sequence comprises a nucleotide sequence at least 95% identical to SEQ ID NO: 6. In some embodiments, the nucleic acid sequence comprises a nucleotide sequence of SEQ ID NO: 6. In some embodiments, the nucleic acid sequence is comprised in a vector. In some embodiments, the vector is an adeno-associated viral (AAV) vector. In some embodiments, the AAV vector is a recombinant AAV (rAAV) vector. In some embodiments, the rAAV vector is selected from the group consisting of AAV2, AAV5, AAV8, AAV9, AAV2/5, AAV 2tYF, and AAV2.7m8. In some embodiments, the vector comprises an AAV capsid protein. In some embodiments, the AAV capsid protein is selected from the group consisting of an AAV2 capsid protein, an AAV2YF capsid protein, an AAV5 capsid protein, an AAV8 capsid protein, an AAV9 capsid protein, and a AAV2.7m8 capsid protein. In some embodiments, the vector further comprises: 1) a 5′ AAV ITR, and 2) a 3′ AAV ITR. In some embodiments, the vector comprises an AAV expression cassette. In some embodiments, the vector further comprises a rhodopsin (RHO) promoter. In some embodiments, the vector further comprises a CBA promoter. In some embodiments, the vector further comprises a human rhodopsin kinase (hGRK1) promoter. In some embodiments, the vector further comprises a CASI promoter. In some embodiments, the vector further comprises a VMD2 promoter. In some embodiments, the composition is for use to treat a disease or disorder. In some embodiments, the disease or disorder comprises a MERTK-mediated disease or disorder. In some embodiments, the MERTK-mediated disease comprises retinitis pigmentosa. In some embodiments, the disease or disorder comprises vision loss. In some embodiments, the composition is administered to a subject prior to onset of the disease or disorder. In some embodiments, the composition is administered to a subject after onset of the disease or disorder. In some embodiments, the composition is administered to a subject in at least one eye. In some embodiments, the composition is administered to a subject by subretinal injection, intravitreal injection, or suprachoroidal injection. In some embodiments, the composition comprises at least about 10⁹ viral particles per mL. In some embodiments, the composition, when administered to a subject, restores at least partial vision to the subject. In some embodiments, the composition, when administered to a subject, improves vision loss of the subject by at least 10% when measured using a visual field test.

Also provided herein are uses of the functional MERTK nucleic acid sequences described herein, or fragments thereof, for the manufacture of a medicament. Also provided herein are kits comprising the functional MERTK nucleic acid sequences described herein, or fragments thereof, and optionally instructions for use.

### BRIEF DESCRIPTION OF THE DRAWINGS

FIG. 1A depicts an exemplary MERTK gene construct. FIG. 1B depicts an exemplary plasmid comprising a MERTK gene construct.

FIG. 2 depicts an outline of a mouse experiment utilizing sub-retinal delivery of AAV2-VMD2-hMERTK.

FIG. 3 depicts exemplary MERTK plasmids. FIG. 3, Panel A depicts VMD2-MERTKwt, which is a MERTK plasmid that contains a vitelliform macular dystrophy-2 (VMD2) (bestrophin-1 (BEST1)) promoter with an unmodified wild type (wt) MERTK sequence, resulting in a 4336 base pair (bp) AAV transfer region. FIG. 3, Panel B depicts CBA-MERTKwt, a MERTK plasmid which contains a cytomegalovirus (CMV) early enhancer and a chicken β-actin (CBA) promoter, and an unmodified wt MERTK sequence resulting in a 4374 bp AAV transfer region. FIG. 3, Panel C depicts VMD2-MERTKopt, a MERTK plasmid which contains a VMD2 promoter with a codon optimized (codop) MERTK sequence, resulting in a 4336 bp AAV transfer region. FIG. 3, Panel D depicts CBA-MERTKopt, a MERTK plasmid which contains a CMV enhancer and CBA promoter with a codop MERTK sequence, resulting in a 4374 bp AAV transfer region.

FIG. 4 is a schematic representation of an exemplary MERTK plasmid experiment.

FIG. 5 shows MERTK gene expression measured using dPCR with 1 nanogram (ng) cDNA input. Gene of interest (GOI) (MERTKwt or MERTKcodop) copies/uL was normalized to loading control RPP30 copies/uL run in the same experiments with the same cDNA input. n=3, p<0.01, ns: not significant, relative to the respective control.

FIG. 6A and FIG. 6B show representative immunoblots showing MERTK from total cell lysates of 293T cells (FIG. 6A) and ARPE-19 cells (FIG. 6B) following 48-hour incubation and 72 hour post reverse transfection. GAPDH was used as a loading control. Sample “A” uses the plasmid from FIG. 3, Panel A; sample “B” uses the plasmid from FIG. 3, Panel B; sample “C” uses the plasmid from FIG. 3, Panel C; and sample “D” uses the plasmid from FIG. 3, Panel D.

FIG. 7A and FIG. 7B show percent cell death, cell density, visualized with phase-contrast microscopy of ARPE19 cells. Sample “A” uses the plasmid from FIG. 3, Panel A; sample “B” uses the plasmid from FIG. 3, Panel B; sample “C” uses the plasmid from FIG. 3, Panel C; and sample “D” uses the plasmid from FIG. 3, Panel D.

FIG. 8 shows the results of an ARPE-19 phagocytosis assay wherein transfected ARPE-19 cells were treated with bPOS-FITC and analyzed using flow cytometry to determine the percentage of FITC+ cells, as a measure of MERTK-driven phagocytic activity. n=3, p<0.00001, relative to control.

FIG. 9 shows a representative immunoblot showing rhodopsin internalization (top) or total rhodopsin (bottom) from cell lysates of ARPE-19 cells following a 30-minute, 60 minute, or 120 minute incubation with bPOS(+). Sample “A” uses the plasmid from FIG. 3, Panel A; sample “B” uses the plasmid from FIG. 3, Panel B; sample “C” uses the plasmid from FIG. 3, Panel C; and sample “D” uses the plasmid from FIG. 3, Panel D.

FIG. 10 shows relative MERTK gene expression in cell culture models of RPE cells, namely iPSC-RPE, ARPE19, differentiated ARPE19 cells. 293T cells were used as a control cell line.

FIG. 11 shows relative native MERTK and codon optimized MERTK gene expression in ARPE19 cells in response to AAV2-VMD2-MERTKwt and AAV2-VMD2-MERTK codop vectors, respectively.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, components, and/or groups, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. The nucleic acids provided herein can comprise RNA, DNA, an DNA/RNA hybrid, a nucleic acid analog, a chemically modified nucleic acid, a chimera composed of two or more nucleic acids or nucleic acid analogs, or any combination thereof. A nucleic acid comprising DNA may be transcribed from DNA into RNA. A nucleic acid comprising DNA may be transcribed from DNA into RNA upon administration into a subject. The nucleic acids provided herein can comprise one or more nucleic acid sequences.

Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, modified nucleic acid sequences, synthetic nucleic acid sequences, and primers. cDNA encoding for a gene or gene fragment referred herein may comprise at least one region encoding for exon sequences without an intervening intron sequence in the genomic equivalent sequence.

The term “percent (%) identity”, “sequence identity”, “percent sequence identity”, or “percent identical” refers to a quantitative measurement of similarity between two sequences (nucleic acids or amino acids).

“Percent identity,” “sequence identity,” “percent sequence identity” or “percent identical” between a query nucleic acid sequence and a subject nucleic acid sequence is the “identities” value, expressed as a percentage, that is calculated using a suitable algorithm or software, such as BLASTN, FASTA, DNASTAR Lasergene, GeneDoc, Bioedit, EMBOSS needle or EMBOSS infoalign, over the entire length of the query sequence after a pair-wise global sequence alignment has been performed using a suitable algorithm or software, such as BLASTN. FASTA, ClustalW, MUSCLE, MAFFT. EMBOSS Needle, T-Coffee, and DNASTAR Lasergene. Importantly, a query nucleic acid sequence may be described by a nucleic acid sequence identified in one or more claims herein.

“Percent identity,” “sequence identity,” “percent sequence identity” or “percent identical” between a query amino acid sequence and a subject amino acid sequence is the “identities” value, expressed as a percentage, that is calculated using a suitable algorithm or software, such as BLASTP, FASTA, DNASTAR Lasergene, GencDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT. EMBOSS Needle, T-Coffee, and DNASTAR Lasergene. Importantly, a query amino acid sequence may be described by an amino acid sequence identified in one or more claims herein. The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

In some embodiments, length of sequence identity comparison may be over the full-length of the genome, the full-length of a gene coding sequence, or a fragment of at least about 500 to 5000 nucleotides. In some embodiments, identity among smaller fragments, e.g. of at least about nine nucleotides, at least about 20 to 24 nucleotides, at least about 28 to 32 nucleotides, at least about 36 or more nucleotides, may also be desired.

A “subject” in need thereof, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease.

The terms “treat,” “treating,” or “treatment,” and its grammatical equivalents as used herein, can include alleviating, abating, or ameliorating at least one symptom of a disease or a condition, preventing additional symptoms, inhibiting the disease or the condition, e.g., delaying, decreasing, suppressing, attenuating, diminishing, arresting, or stabilizing the development or progression of a disease or the condition, relieving the disease or the condition, causing regression of the disease or the condition, relieving a condition caused by the disease or the condition, reducing disease severity, or stopping the symptoms of the disease or the condition either prophylactically and/or therapeutically. “Treating” also includes lessening the frequency of occurrence or recurrence, or the severity, of any symptoms or other ill effects related to a disease or condition and/or the side effects associated with the disease or condition. “Treating” does not necessarily require curative results. It is appreciated that, although not precluded, treating a disorder or condition also does not require that the disorder, condition, or symptoms associated therewith be completely eliminated. The term “treating” encompasses the concept of “managing” which refers to reducing the severity of a particular disease or disorder in a patient or delaying its recurrence, e.g., lengthening the period of remission in a patient who had suffered from the disease. “Treating” may refer to the application or administration or a composition to a subject after the onset, or suspected onset, of a disease or condition.

The term “treating” further encompasses the concept of “prevent,” “preventing,” and “prevention.” The terms “prevent,” “preventing,” and “prevention,” as used herein, refer to a decrease in the occurrence of pathology of a condition in a subject, who does not have, but is at risk of or susceptible to developing a disease or condition. The prevention may be complete, e.g., the total absence of pathology of a condition in a subject. The prevention may also be partial, such that the occurrence of pathology of a condition in a subject is less than that which would have occurred without the present disclosure.

Administering” and its grammatical equivalents as used herein can refer to providing pharmaceutical compositions described herein to a subject or a patient. Conventional methods, known to those of ordinary skill in the art of medicine, can be used to administer the composition to the subject, depending upon the type of disease to be treated or the site of the disease. For example, the composition can be administered, e.g., orally, parenterally, topically, or via an implanted reservoir. In some embodiments, the composition is administered via an injection suitable for delivery to the eye (e.g., via an intravitreal, a subretinal, or a suprachoroidal injection). One or more such routes can be employed.

### MERTK Gene Therapy

MERTK is a receptor tyrosine kinase that transduces signals from the extracellular matrix into the cytoplasm by binding to several ligands (e.g., LGALS3, TUB, TULP1 or GAS6). MERTK regulates many physiological processes including cell survival, migration, differentiation, and phagocytosis of apoptotic cells (efferocytosis). Ligand binding at the cell surface induces autophosphorylation of MERTK on its intracellular domain that provides docking sites for downstream signaling molecules. Following activation by a ligand, MERTK interacts with GRB2 or PLCG2 and induces phosphorylation of MAPKI, MAPK2, FAK/PTK2 or RACI. MERTK signaling plays a role in various processes such as macrophage clearance of apoptotic cells, platelet aggregation, cytoskeleton reorganization and engulfment. The MERTK gene is expressed in several hematopoietic tissues (e.g., macrophages), epithelial tissues (e.g., retinal pigment epithelium tissues), and reproductive tissues.

In the eye, MERTK functions in the retinal pigment epithelium (RPE) as a regulator of photoreceptor outer segments (e.g., rod outer segment fragment phagocytosis). Phagocytosis of photoreceptor outer segments (POS) involves three primary steps: 1) binding of POS to integrins, 2) the binding is transduced into an intracellular signal through an increase in intracellular inositol 1,4,5-triphosphate (InsP3), leading to 3) POS ingestion facilitated by the macrophage receptor CD36 and signaling by MERTK and focal adhesion kinase (FAK). MERTK also plays an important role in the inhibition of Toll-like receptor (TLR)-mediated innate immune response by activating STAT1, which selectively induces production of suppressors of cytokine signaling SOCS1 and SOCS3. Mutations in MERTK can be associated with disruption of the retinal pigment epithelium (RPE) phagocytosis pathway and onset of vision-related diseases and disorders (e.g., autosomal recessive retinitis pigmentosa (RP)).

The present methods include sequences encoding human MERTK, or biologically active fragments thereof. In some embodiments, the sequence encoding MERTK comprises SEQ ID NO: 1, or a sequence that is at least about 70%, at least about 75%, at least about 80%, at least about 85%, at least about 90%, at least about 95%, at least about 97%, at least about 98%, or at least about 99% identical to SEQ ID NO: 1. In some embodiments, the sequence encoding MERTK comprises SEQ ID NO: 2, or a sequence that is at least about 70%, at least about 75%, at least about 80%, at least about 85%, at least about 90%, at least about 95%, at least about 97%, at least about 98%, or at least about 99% identical to SEQ ID NO: 2. Table 1 provides exemplary sequences of MERTK.

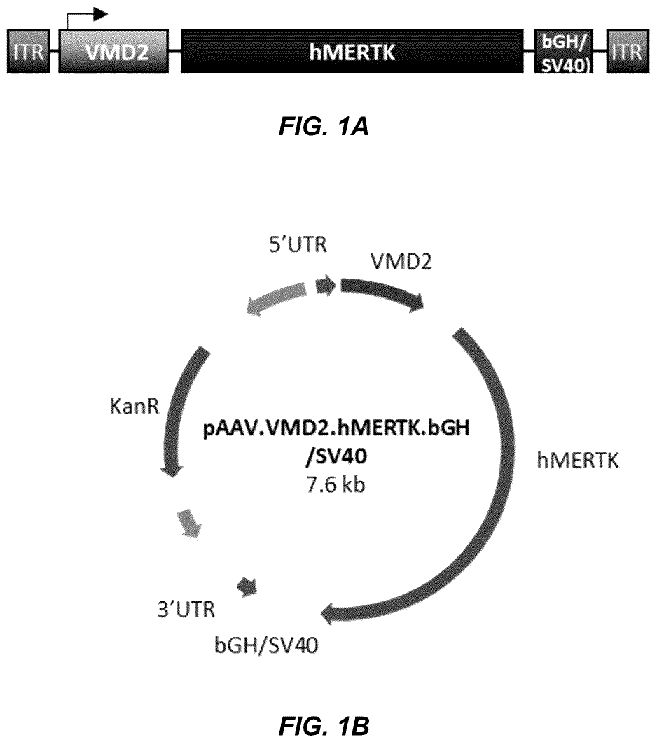

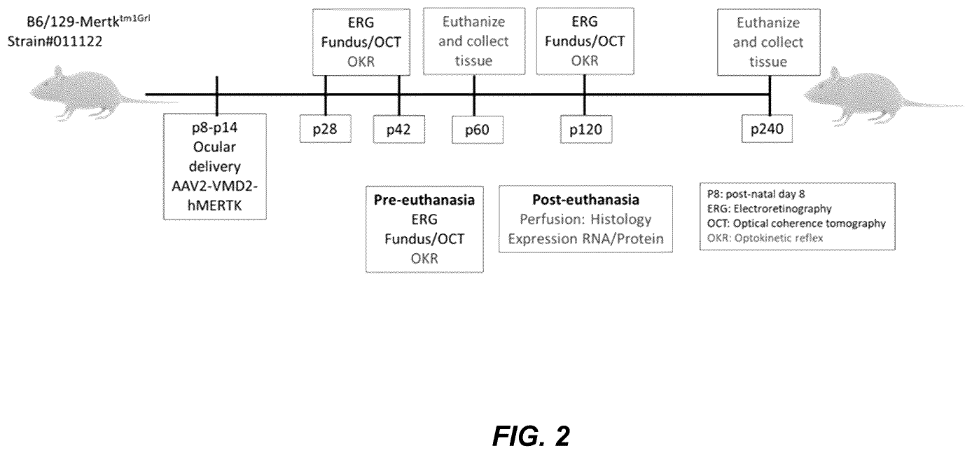

In some embodiments, the sequence encoding MERTK can be wild type. In some embodiments, the sequence encoding MERTK can be codon optimized so that it can be more efficiently translated into an amino acid sequence. In some embodiments, the MERTK sequence can take the form of FIG. 1A.

In one aspect, a codon optimized, engineered nucleic acid comprising a nucleic acid sequence encoding human MERTK is provided. In some embodiments, the MERTK coding nucleic acid sequence is codon optimized for expression in humans. In some embodiments, the codon optimized MERTK coding sequence has less than about 80% identity, preferably about 76% identity or less to the wildtype or native MERTK coding sequence (SEQ ID NO: 1). In some embodiments, the codon optimized MERTK coding sequence has about 76% identity with the native MERTK coding sequence of SEQ ID NO: 1. In some embodiments, the codon optimized MERTK coding sequence has about 64% identity with the native MERTK coding sequence of SEQ ID NO: 1. In some embodiments, the codon optimized MERTK coding sequence is characterized by improved translation rate as compared to the wildtype or native MERTK following delivery in a plasmid or a recombinant viral vector (e.g., rAAV). In some embodiments, the codon optimized MERTK coding sequence shares less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or less identity to the wildtype MERTK coding sequence of SEQ ID NO: 1. In some embodiments, the codon optimized nucleic acid sequence comprises any one of SEQ ID NOs: 3-6. In some embodiments, the codon optimized nucleic acid sequence comprises SEQ ID NO: 3. In some embodiments, the codon optimized nucleic acid sequence comprises SEQ ID NO: 4. In some embodiments, the codon optimized nucleic acid sequence comprises SEQ ID NO: 5. In some embodiments, the codon optimized nucleic acid sequence comprises SEQ ID NO: 6. In some embodiments, the codon optimized nucleic acid sequence comprises a variant of SEQ ID NOs: 3-6. In some embodiments, the codon optimized nucleic acid sequence is a sequence sharing about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or greater identity with any one of SEQ ID NOs: 3-6.

In some embodiments, the nucleic acid comprising a nucleotide sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identical to any one of SEQ ID NOs: 3-6. In some embodiments, the nucleic acid comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NO: 3. In some embodiments, the nucleic acid comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NO: 4. In some embodiments, the nucleic acid comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NO: 5. In some embodiments, the nucleic acid comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NO: 6.

In some embodiments, a codon optimized MERTK comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NOs: 2.

In some embodiments, the MERTK construct comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 7. In some embodiments, the MERTK construct comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 8. In some embodiments, the MERTK construct comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 9. In some embodiments, the MERTK construct comprises a sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 10.

In some embodiments, the nucleic acid comprising the MERTK coding nucleic acid sequence can comprise a non-coding sequence or a regulatory sequence. In some embodiments, the MERTK coding nucleic acid sequence further comprises a non-coding sequence or a regulatory sequence. In some embodiments, the non-coding sequence or regulatory sequence can comprise a 5′ untranslated region (5′-UTR), a 3′ untranslated region (3′-UTR), a promoter, an enhancer, a polyadenylation (poly-A) sequence, or a cis-regulatory element. In some embodiments, the cis-regulatory element comprises a silencer, an operator, a woodchuck hepatitis virus post-transcriptional regulatory element (WPRE), or a minute virus of mice (MVM) intron. In some embodiments, the non-coding sequence or regulatory sequence can comprise a portion of an exon or a portion of an intron. In some embodiments, polyadenylation sequence comprises a bovine growth hormone polyadenylation signal (bGHpA).

In some embodiments, the non-coding sequence or the regulatory sequence can comprise a promoter. In some cases, the promoter can include, but is not limited to, a Cytomegalovirus (CMV) promoter, a hybrid CMV enhancer/chicken β-acting (CBA) promoter, a CAG promoter, a rhodopsin (RHO) promoter (e.g., a human rhodopsin (hRHO) promoter, a Rho 194 promoter), a CASI promoter, a rhodopsin kinase promoter (e.g., a GRK1 promoter), an interphotoreceptor retinoid binding protein (IRBP) promoter, a red opsin promoter, a vitelliform macular dystrophy (VMD2) promoter, and a cadherin promoter (e.g., a CDH5 promoter or a CD144 promoter). Vectors can include promoters that drive expression in many cell types (e.g., CAG, CMV, or CASI). Alternatively, vectors can include promoters that drive expression in specific cell types, such as photoreceptor cells (RHO, rhodopsin kinase (GRK1), cone arrestin (CAR)) or RPE cells (e.g., promotors for RPE-specific proteins such as VMD2, RPE65, RLBP1, RGR, or TIMP3). In some embodiments, the promoter can be CAG, CAGGS, CASI, CBh, GFAP, TRE3G, SMVP, Spc512, HI/TO, CK7-miniCMV, pICAM2, HCRhAATp, or a Tight promoter. Synthetic promoters ProC1 and ProD5 can also be used. In some embodiments, the promoter can be a hybrid promoter. For example, a promoter can be a CASI promoter, which includes a CMV enhancer and a CBA promoter. In some cases, a CASI promoter further comprises a ubiquitin (UBC) enhancer.

In some embodiments, the non-coding sequence or the regulatory sequence can comprise an enhancer or a portion thereof. An enhancer can include, but is not limited to a CMV enhancer, a ubiquitin enhancer (e.g., a UBC enhancer), a CBA/CAG enhancer, a RHO enhancer, a rhodopsin kinase enhancer, an IRBP enhancer, a red opsin enhancer, a VMD2 enhancer, a CASI enhancer, and a cadherin enhancer.

TABLE 1
MERTK Sequences

| SEQ ID | Sequence | |
|---|---|---|
| NO | Name | Sequence |
| 1 | MERTK | ATGGGGCCGGCCCCGCTGCCGCTGCTGCTGGGCCTCTTCCTCCCC |
| | nucleic | GCGCTCTGGCGTAGAGCTATCACTGAGGCAAGGGAAGAAGCCA |
| | acid | AGCCTTACCCGCTATTCCCGGGACCTTTTCCAGGGAGCCTGCAA |
| | sequence | ACTGACCACACACCGCTGTTATCCCTTCCTCACGCCAGTGGGTAC |
| | | CAGCCTGCCTTGATGTTTTCACCAACCCAGCCTGGAAGACCACA |
| | | TACAGGAAACGTAGCCATTCCCCAGGTGACCTCTGTCGAATCAA |
| | | AGCCCCTACCGCCTCTTGCCTTCAAACACACAGTTGGACACATA |
| | | ATACTTTCTGAACATAAAGGTGTCAAATTTAATTGCTCAATCAGT |
| | | GTACCTAATATATACCAGGACACCACAATTTCTTGGTGGAAAGA |
| | | TGGGAAGGAATTGCTTGGGGCACATCATGCAATTACACAGTTTT |
| | | ATCCAGATGATGAAGTTACAGCAATAATCGCTTCCTTCAGCATA |
| | | ACCAGTGTGCAGCGTTCAGACAATGGGTCGTATATCTGTAAGAT |
| | | GAAAATAAACAATGAAGAGATCGTGTCTGATCCCATCTACATCG |
| | | AAGTACAAGGACTTCCTCACTTTACTAAGCAGCCTGAGAGCATG |
| | | AATGTCACCAGAAACACAGCCTTCAACCTCACCTGTCAGGCTGT |
| | | GGGCCCGCCTGAGCCCGTCAACATTTTCTGGGTTCAAAACAGTA |
| | | GCCGTGTTAACGAACAGCCTGAAAAATCCCCCTCCGTGCTAACT |
| | | GTTCCAGGCCTGACGGAGATGGCGGTCTTCAGTTGTGAGGCCCA |
| | | CAATGACAAAGGGCTGACCGTGTCCAAGGGAGTGCAGATCAAC |
| | | ATCAAAGCAATTCCCTCCCCACCAACTGAAGTCAGCATCCGTAA |
| | | CAGCACTGCACACAGCATTCTGATCTCCTGGGTTCCTGGTTTTGA |
| | | TGGATACTCCCCGTTCAGGAATTGCAGCATTCAGGTCAAGGAAG |
| | | CTGATCCGCTGAGTAATGGCTCAGTCATGATTTTTAACACCTCTG |
| | | CCTTACCACATCTGTACCAAATCAAGCAGCTGCAAGCCCTGGCT |
| | | AATTACAGCATTGGTGTTTCCTGCATGAATGAAATAGGCTGGTCT |
| | | GCAGTGAGCCCTTGGATTCTAGCCAGCACGACTGAAGGAGCCCC |
| | | ATCAGTAGCACCTTTAAATGTCACTGTGTTTCTGAATGAATCTAG |
| | | TGATAATGTGGACATCAGATGGATGAAGCCTCCGACTAAGCAGC |
| | | AGGATGGAGAACTGGTGGGCTACCGGATATCCCACGTGTGGCAG |
| | | AGTGCAGGGATTTCCAAAGAGCTCTTGGAGGAAGTTGGCCAGAA |
| | | TGGCAGCCGAGCTCGGATCTCTGTTCAAGTCCACAATGCTACGT |
| | | GCACAGTGAGGATTGCAGCCGTCACCAGAGGGGGAGTTGGGCC |
| | | CTTCAGTGATCCAGTGAAAATATTTATCCCTGCACACGGTTGGGT |
| | | AGATTATGCCCCCTCTTCAACTCCGGCGCCTGGCAACGCAGATC |
| | | CTGTGCTCATCATCTTTGGCTGCTTTTGTGGATTTATTTTGATTGG |
| | | GTTGATTTTATACATCTCCTTGGCCATCAGAAAAAGAGTCCAGG |
| | | AGACAAAGTTTGGGAATGCATTCACAGAGGAGGATTCTGAATTA |
| | | GTGGTGAATTATATAGCAAAGAAATCCTTCTGTCGGCGAGCCAT |
| | | TGAACTTACCTTACATAGCTTGGGAGTCAGTGAGGAACTACAAA |
| | | ATAAACTAGAAGATGTTGTGATTGACAGGAATCTTCTAATTCTTG |
| | | GAAAAATTCTGGGTGAAGGAGAGTTTGGGTCTGTAATGGAAGGA |
| | | AATCTTAAGCAGGAAGATGGGACCTCTCTGAAAGTGGCAGTGAA |
| | | GACCATGAAGTTGGACAACTCTTCACAGCGGGAGATCGAGGAGT |
| | | TTCTCAGTGAGGCAGCGTGCATGAAAGACTTCAGCCACCCAAAT |
| | | GTCATTCGACTTCTAGGTGTGTGTATAGAAATGAGCTCTCAAGG |
| | | CATCCCAAAGCCCATGGTAATTTTACCCTTCATGAAATACGGGG |
| | | ACCTGCATACTTACTTACTTTATTCCCGATTGGAGACAGGACCAA |
| | | AGCATATTCCTCTGCAGACACTATTGAAGTTCATGGTGGATATTG |
| | | CCCTGGGAATGGAGTATCTGAGCAACAGGAATTTTCTTCATCGA |
| | | GATTTAGCTGCTCGAAACTGCATGTTGCGAGATGACATGACTGT |
| | | CTGTGTTGCGGACTTCGGCCTCTCTAAGAAGATTTACAGTGGCG |
| | | ATTATTACCGCCAAGGCCGCATTGCTAAGATGCCTGTTAAATGG |
| | | ATCGCCATAGAAAGTCTTGCAGACCGAGTCTACACAAGTAAAAG |
| | | TGATGTGTGGGCATTTGGCGTGACCATGTGGGAAATAGCTACGC |
| | | GGGGAATGACTCCCTATCCTGGGGTCCAGAACCATGAGATGTAT |
| | | GACTATCTTCTCCATGGCCACAGGTTGAAGCAGCCCGAAGACTG |
| | | CCTGGATGAACTGTATGAAATAATGTACTCTTGCTGGAGAACCG |
| | | ATCCCTTAGACCGCCCCACCTTTTCAGTATTGAGGCTGCAGCTAG |
| | | AAAAACTCTTAGAAAGTTTGCCTGACGTTCGGAACCAAGCAGAC |
| | | GTTATTTACGTCAATACACAGTTGCTGGAGAGCTCTGAGGGCCT |
| | | GGCCCAGGGCTCCACCCTTGCTCCACTGGACTTGAACATCGACC |
| | | CTGACTCTATAATTGCCTCCTGCACTCCCCGCGCTGCCATCAGTG |
| | | TGGTCACAGCAGAAGTTCATGACAGCAAACCTCATGAAGGACGG |
| | | TACATCCTGAATGGGGGCAGTGAGGAATGGGAAGATCTGACTTC |
| | | TGCCCCCTCTGCTGCAGTCACAGCTGAAAAGAACAGTGTTTTAC |
| | | CGGGGGAGAGACTTGTTAGGAATGGGGTCTCCTGGTCCCATTCG |
| | | AGCATGCTGCCCTTGGGAAGCTCATTGCCCGATGAACTTTTGTTT |
| | | GCTGACGACTCCTCAGAAGGCTCAGAAGTCCTGATGTGA |
| 2 | MERTK | MGPAPLPLLLGLFLPALWRRAITEAREEAKPYPLFPGPFPGSLQTDH |
| | amino | TPLLSLPHASGYQPALMFSPTQPGRPHTGNVAIPQVTSVESKPLPPL |
| | acid | AFKHTVGHIILSEHKGVKFNCSISVPNIYQDTTISWWKDGKELLGAH |
| | sequence | HAITQFYPDDEVTAIIASFSITSVQRSDNGSYICKMKINNEEIVSDPIYI |
| | | EVQGLPHFTKQPESMNVTRNTAFNLTCQAVGPPEPVNIFWVQNSSR |
| | | VNEQPEKSPSVLTVPGLTEMAVFSCEAHNDKGLTVSKGVQINIKAIP |
| | | SPPTEVSIRNSTAHSILISWVPGFDGYSPFRNCSIQVKEADPLSNGSV |
| | | MIFNTSALPHLYQIKQLQALANYSIGVSCMNEIGWSAVSPWILASTT |
| | | EGAPSVAPLNVTVFLNESSDNVDIRWMKPPTKQQDGELVGYRISHV |
| | | WQSAGISKELLEEVGQNGSRARISVQVHNATCTVRIAAVTRGGVGP |
| | | FSDPVKIFIPAHGWVDYAPSSTPAPGNADPVLIIFGCFCGFILIGLILYI |
| | | SLAIRKRVQETKFGNAFTEEDSELVVNYIAKKSFCRRAIELTLHSLG |
| | | VSEELQNKLEDVVIDRNLLILGKILGEGEFGSVMEGNLKQEDGTSLK |
| | | VAVKTMKLDNSSQREIEEFLSEAACMKDFSHPNVIRLLGVCIEMSSQ |
| | | GIPKPMVILPFMKYGDLHTYLLYSRLETGPKHIPLQTLLKFMVDIAL |
| | | GMEYLSNRNFLHRDLAARNCMLRDDMTVCVADFGLSKKIYSGDY |
| | | YRQGRIAKMPVKWIAIESLADRVYTSKSDVWAFGVTMWEIATRGM |
| | | TPYPGVQNHEMYDYLLHGHRLKQPEDCLDELYEIMYSCWRTDPLD |
| | | RPTFSVLRLQLEKLLESLPDVRNQADVIYVNTQLLESSEGLAQGSTL |
| | | APLDLNIDPDSIIASCTPRAAISVVTAEVHDSKPHEGRYILNGGSEEW |
| | | EDLTSAPSAAVTAEKNSVLPGERLVRNGVSWSHSSMLPLGSSLPDE |
| | | LLFADDSSEGSEVLM |
| 3 | MERTK-1 | ATGGGTCCCGCTCCTCTGCCTCTGCTGCTGGGCCTGTTCCTCCCT |
| | | GCCCTGTGGCGGCGGGCCATCACCGAGGCCAGAGAAGAGGCCA |
| | | AGCCCTACCCTCTGTTCCCCGGCCCTTTCCCCGGATCCCTGCAAA |
| | | CAGACCACACCCCTCTGCTCAGCCTGCCTCATGCCTCCGGCTATC |
| | | AGCCTGCCCTGATGTTCAGCCCTACTCAGCCTGGCCGGCCTCACA |
| | | CCGGCAATGTGGCCATCCCTCAGGTGACATCCGTGGAAAGCAAA |
| | | CCCCTGCCTCCTCTTGCCTTTAAGCACACAGTGGGCCACATTATC |
| | | CTGAGCGAGCACAAGGGCGTGAAGTTCAACTGCAGTATATCTGT |
| | | GCCGAACATCTATCAAGACACAACCATCAGCTGGTGGAAGGACG |
| | | GAAAAGAACTGCTGGGAGCCCACCACGCCATCACCCAGTTCTAC |
| | | CCTGATGATGAAGTGACAGCCATCATCGCCAGCTTCAGCATCAC |
| | | CAGCGTTCAAAGAAGCGACAACGGCAGCTACATCTGCAAGATG |
| | | AAGATCAATAACGAAGAGATCGTGAGCGATCCTATCTACATTGA |
| | | GGTCCAGGGACTGCCCCACTTCACCAAGCAGCCAGAAAGCATGA |
| | | ACGTTACAAGAAATACTGCCTTCAACCTGACCTGCCAGGCTGTG |
| | | GGCCCACCTGAGCCAGTGAATATCTTTTGGGTGCAAAATTCCTCT |
| | | AGAGTAAATGAGCAGCCTGAAAAGTCTCCTAGCGTGCTGACCGT |
| | | GCCTGGCCTGACCGAGATGGCCGTCTTCAGCTGCGAGGCTCACA |
| | | ACGATAAGGGACTGACAGTGAGCAAGGGCGTTCAAATCAACAT |
| | | CAAGGCCATCCCCAGCCCACCAACCGAAGTGTCCATCAGAAATA |
| | | GCACCGCCCATAGCATCCTGATTAGCTGGGTGCCTGGTTTTGACG |
| | | GCTACTCCCCCTTCCGGAACTGTAGCATCCAGGTGAAGGAAGCC |
| | | GATCCGCTGTCAAATGGATCTGTGATGATCTTCAACACCAGCGC |
| | | CCTGCCTCATCTGTACCAGATCAAGCAGCTGCAAGCTCTGGCCA |
| | | ACTACAGCATCGGCGTGTCCTGCATGAACGAGATCGGATGGTCC |
| | | GCTGTGTCCCCATGGATTCTTGCCAGCACCACCGAGGGCGCCCC |
| | | TTCTGTTGCCCCACTGAACGTGACCGTATTTCTGAACGAGAGCA |
| | | GCGACAACGTGGACATCAGATGGATGAAGCCACCTACCAAGCA |
| | | GCAGGATGGCGAGCTGGTGGGCTACAGGATCTCCCACGTGTGGC |
| | | AGAGCGCCGGCATCAGCAAGGAGCTGCTGGAAGAGGTCGGCCA |
| | | GAATGGCAGCAGAGCCCGGATCAGCGTCCAGGTTCACAACGCA |
| | | ACCTGTACAGTCAGAATCGCCGCCGTGACAAGAGGCGGCGTGGG |
| | | CCCCTTCAGCGATCCTGTTAAAATCTTCATCCCCGCCCACGGATG |
| | | GGTCGACTACGCCCCTAGCAGCACCCCTGCTCCCGGCAACGCCG |
| | | ACCCTGTGCTGATCATCTTCGGATGCTTCTGCGGCTTTATCCTGA |
| | | TCGGCCTGATCCTGTACATCTCCCTGGCCATCCGCAAGCGGGTGC |
| | | AGGAGACAAAGTTCGGCAACGCCTTCACAGAAGAGGATTCCGA |
| | | ACTGGTGGTGAACTACATCGCTAAAAAGAGCTTCTGCAGAAGAG |
| | | CCATCGAGCTGACACTGCACAGCCTGGGAGTGTCTGAGGAACTG |
| | | CAGAACAAGCTGGAAGATGTGGTCATCGACAGAAACCTGCTCAT |
| | | ACTGGGCAAAATTCTGGGCGAAGGAGAGTTCGGCAGCGTGATG |
| | | GAAGGCAACCTGAAGCAGGAGGACGGGACCAGCCTGAAGGTGG |
| | | CTGTGAAAACCATGAAGCTGGACAACAGCTCCCAGAGGGAGAT |
| | | CGAGGAATTCCTGAGTGAAGCCGCCTGCATGAAAGACTTTTCTC |
| | | ACCCCAACGTGATTAGACTGCTGGGCGTGTGTATCGAAATGAGC |
| | | TCTCAGGGCATCCCCAAGCCCATGGTGATCCTGCCCTTCATGAA |
| | | GTACGGCGATCTGCACACCTACCTGCTGTATTCTAGACTGGAGA |
| | | CAGGCCCTAAGCACATCCCTCTGCAGACCCTGCTGAAATTCATG |
| | | GTGGATATCGCCCTGGGCATGGAATACCTGAGCAACAGAAACTT |
| | | CCTGCACAGAGACCTTGCTGCCCGGAACTGCATGCTGCGGGACG |
| | | ACATGACGGTGTGCGTGGCCGACTTCGGACTGAGCAAGAAAATC |
| | | TACAGCGGCGACTACTACAGACAGGGCAGAATCGCTAAGATGCC |
| | | TGTGAAGTGGATCGCCATCGAGTCTCTGGCCGATAGAGTGTACA |
| | | CCTCGAAGAGCGATGTGTGGGCCTTTGGAGTTACAATGTGGGAG |
| | | ATCGCCACCAGAGGCATGACCCCTTATCCTGGCGTGCAGAACCA |
| | | CGAGATGTACGACTACCTGCTGCACGGGCACCGACTGAAACAGC |
| | | CCGAGGACTGCCTGGACGAGCTGTACGAGATCATGTACAGCTGT |
| | | TGGCGGACCGACCCTCTGGATCGCCCCACATTTAGCGTGCTGCG |
| | | GCTGCAGCTGGAAAAGCTGCTCGAGAGTCTGCCCGACGTGCGGA |
| | | ACCAGGCCGATGTGATTTACGTGAACACCCAGCTGCTGGAAAGC |
| | | TCCGAGGGCCTGGCCCAGGGGAGCACCCTGGCTCCTCTGGACCT |
| | | GAATATCGACCCAGACAGCATTATCGCCTCTTGTACCCCGAGAG |
| | | CCGCTATCAGCGTGGTGACCGCCGAGGTGCACGACAGCAAGCCC |
| | | CACGAGGGCAGATACATCCTGAACGGAGGCTCTGAGGAGTGGG |
| | | AAGATCTCACAAGCGCTCCTTCTGCCGCCGTGACCGCCGAGAAG |
| | | AACTCCGTGCTACCTGGCGAGAGACTTGTGCGGAACGGCGTCAG |
| | | CTGGAGCCACAGCAGCATGCTGCCTCTGGGCAGCAGTCTTCCTG |
| | | ATGAACTGCTCTTCGCCGACGACAGCAGCGAGGGTTCAGAGGTG |
| | | CTGATGTGA |
| 4 | MERTK-2 | ATGGGACCAGCTCCTCTGCCTCTGCTGCTGGGACTGTTTCTGCCA |
| | | GCTCTCTGGAGGAGGGCCATTACAGAGGCCAGAGAGGAGGCCA |
| | | AGCCTTACCCTCTGTTTCCAGGCCCTTTCCCCGGATCTCTGCAGA |
| | | CCGATCACACCCCTCTGCTGTCTCTGCCTCACGCTAGCGGATACC |
| | | AGCCAGCCCTGATGTTTAGCCCTACCCAGCCAGGCAGACCTCAC |
| | | ACAGGAAACGTGGCCATCCCTCAGGTCACAAGCGTGGAGAGCA |
| | | AGCCTCTGCCTCCTCTGGCCTTTAAGCACACCGTGGGACACATCA |
| | | TCCTGAGCGAGCACAAGGGCGTGAAGTTCAATTGCAGCATCAGC |
| | | GTGCCCAACATCTACCAGGACACCACCATCAGTTGGTGGAAGGA |
| | | CGGCAAGGAACTGCTGGGAGCTCATCACGCCATCACCCAGTTCT |
| | | ACCCCGACGACGAAGTGACCGCCATCATCGCCAGCTTCAGCATC |
| | | ACCAGCGTGCAGAGGAGCGACAACGGCAGCTACATCTGCAAGA |
| | | TGAAGATCAACAACGAGGAGATCGTGTCCGACCCCATCTACATC |
| | | GAGGTGCAGGGACTGCCTCACTTCACCAAGCAGCCAGAGAGCAT |
| | | GAACGTGACCCGGAACACCGCCTTCAACCTGACTTGCCAGGCAG |
| | | TGGGACCTCCAGAGCCCGTGAACATCTTCTGGGTGCAGAACAGC |
| | | AGCCGCGTGAACGAACAGCCAGAGAAGAGCCCTAGCGTGCTGA |
| | | CAGTGCCAGGACTGACAGAGATGGCCGTGTTCTCTTGCGAGGCC |
| | | CACAACGACAAGGGACTGACCGTGTCCAAGGGCGTGCAGATCA |
| | | ACATCAAGGCCATCCCTAGCCCTCCTACAGAGGTGTCCATCAGG |
| | | AACAGCACCGCCCACAGCATCCTGATCTCTTGGGTGCCAGGCTT |
| | | CGACGGATACAGCCCCTTCCGGAATTGCAGCATCCAGGTGAAGG |
| | | AGGCCGATCCTCTGAGCAACGGAAGCGTGATGATCTTCAACACC |
| | | AGCGCCCTGCCCCACCTGTACCAGATCAAGCAGCTGCAGGCCCT |
| | | GGCTAACTACAGCATCGGCGTGTCTTGCATGAACGAGATCGGTT |
| | | GGAGCGCCGTGAGCCCTTGGATTCTGGCCAGCACAACCGAGGGA |
| | | GCTCCATCAGTGGCTCCACTGAACGTGACCGTGTTCCTGAACGA |
| | | GAGCAGCGACAACGTGGACATCCGCTGGATGAAGCCTCCCACAA |
| | | AGCAGCAGGACGGAGAACTCGTGGGCTACAGAATCAGCCACGT |
| | | CTGGCAGAGTGCCGGAATCAGCAAGGAGCTGCTGGAGGAAGTG |
| | | GGCCAGAACGGCTCTAGAGCCAGGATCAGCGTGCAGGTGCACA |
| | | ACGCCACTTGCACCGTGAGAATCGCCGCAGTGACAAGAGGAGG |
| | | AGTGGGACCCTTCAGCGACCCCGTGAAGATCTTCATCCCAGCTC |
| | | ACGGTTGGGTGGATTACGCTCCTAGCAGCACACCAGCTCCAGGA |
| | | AACGCCGATCCCGTGCTGATCATCTTCGGCTGCTTCTGCGGCTTC |
| | | ATCCTGATCGGCCTGATCCTGTACATCAGCCTGGCCATCCGGAA |
| | | GAGAGTGCAGGAGACCAAGTTCGGCAACGCCTTCACCGAGGAG |
| | | GACAGCGAGCTGGTCGTGAACTACATCGCCAAGAAGAGCTTTTG |
| | | CCGGAGGGCCATCGAGCTGACACTGCACTCTCTGGGAGTGTCCG |
| | | AGGAGCTGCAGAACAAGCTGGAGGACGTCGTGATCGACCGGAA |
| | | CCTGCTGATTCTGGGCAAGATCCTGGGCGAGGGAGAGTTTGGCA |
| | | GCGTGATGGAGGGCAACCTGAAGCAGGAGGACGGAACAAGCCT |
| | | GAAGGTGGCCGTGAAGACCATGAAGCTGGACAACAGCAGCCAG |
| | | CGGGAGATCGAAGAGTTCCTGAGCGAGGCCGCTTGCATGAAGG |
| | | ACTTCAGCCACCCCAACGTGATCAGGCTGCTCGGCGTCTGTATC |
| | | GAGATGAGCAGCCAGGGAATCCCCAAGCCCATGGTCATCCTGCC |
| | | CTTCATGAAGTACGGCGACCTGCACACCTACCTGCTGTACAGCA |
| | | GGCTGGAGACCGGACCTAAGCACATCCCTCTGCAGACCCTGCTG |
| | | AAGTTCATGGTGGACATCGCCCTGGGCATGGAGTACCTGAGCAA |
| | | CCGGAACTTCCTGCACAGAGACCTGGCCGCTCGGAATTGCATGC |
| | | TGAGAGACGACATGACCGTCTGCGTGGCAGACTTCGGCCTGAGC |
| | | AAGAAGATCTACAGCGGCGACTACTACAGACAGGGCAGAATCG |
| | | CCAAGATGCCCGTCAAGTGGATCGCCATCGAGAGCCTGGCCGAT |
| | | AGGGTGTACACCAGCAAGAGCGACGTCTGGGCTTTCGGAGTGAC |
| | | CATGTGGGAGATCGCCACCAGAGGCATGACACCTTACCCAGGCG |
| | | TGCAGAACCACGAGATGTACGACTACCTGCTGCACGGCCACAGA |
| | | CTGAAGCAGCCAGAGGATTGCCTGGACGAGCTGTACGAGATCAT |
| | | GTACTCTTGTTGGCGGACCGACCCTCTGGACAGACCTACCTTCAG |
| | | CGTGCTGAGGCTGCAGCTGGAAAAGCTGCTGGAGAGCCTGCCAG |
| | | ACGTCAGAAATCAGGCCGACGTGATCTACGTGAACACCCAGCTG |
| | | CTGGAGAGCAGCGAAGGACTGGCTCAGGGCTCTACACTGGCCCC |
| | | TCTGGATCTGAACATCGACCCCGATAGCATCATCGCCTCTTGCAC |
| | | CCCTAGAGCCGCTATCAGCGTGGTGACAGCCGAAGTGCACGACA |
| | | GCAAGCCTCACGAGGGACGCTACATCCTGAACGGAGGAAGCGA |
| | | GGAGTGGGAGGATCTGACAAGCGCTCCTTCTGCTGCCGTGACAG |
| | | CCGAAAAGAACAGCGTGCTGCCAGGAGAGAGGCTCGTGAGAAA |
| | | CGGCGTGTCTTGGAGCCACAGCAGCATGCTGCCTCTGGGAAGCT |
| | | CTCTGCCAGACGAGCTGCTCTTTGCCGACGATAGCAGCGAGGGA |
| | | AGCGAGGTGCTGATGTAG |
| 5 | MERTK-3 | ATGGGACCTGCTCCTCTGCCTCTGCTGCTGGGACTGTTTCTGCCT |
| | | GCTCTTTGGCGGAGAGCCATCACCGAGGCCAGAGAGGAAGCCA |
| | | AGCCTTATCCTCTGTTCCCCGGACCTTTTCCAGGCAGCCTGCAGA |
| | | CCGATCACACCCCTTTGCTGTCTCTGCCTCACGCCTCTGGCTATC |
| | | AGCCCGCTCTGATGTTCAGCCCCACACAGCCAGGCAGACCTCAC |
| | | ACAGGCAATGTGGCCATTCCTCAAGTGACCAGCGTGGAAAGCAA |
| | | GCCCTTGCCTCCTCTGGCCTTCAAGCACACAGTGGGCCACATCAT |
| | | CCTGAGCGAGCACAAGGGCGTGAAGTTCAACTGCAGCATCAGCG |
| | | TGCCCAACATCTACCAGGACACCACCATCAGCTGGTGGAAGGAC |
| | | GGCAAAGAACTGCTGGGAGCCCACCACGCCATCACACAGTTCTA |
| | | CCCCGACGATGAAGTGACCGCCATCATTGCCAGCTTCAGCATCA |
| | | CCAGCGTGCAGAGAAGCGACAACGGCAGCTACATCTGCAAGAT |
| | | GAAGATCAACAACGAGGAAATCGTCAGCGACCCCATCTACATCG |
| | | AGGTGCAGGGCCTGCCTCACTTCACCAAGCAGCCCGAGAGCATG |
| | | AACGTGACCAGAAACACCGCCTTCAACCTGACCTGTCAGGCCGT |
| | | GGGACCTCCTGAGCCTGTGAACATCTTCTGGGTGCAGAACAGCT |
| | | CCAGAGTGAACGAGCAGCCTGAGAAGTCCCCTAGCGTGCTGACA |
| | | GTGCCTGGACTGACAGAGATGGCCGTGTTTTCTTGCGAGGCCCA |
| | | CAACGACAAGGGCCTGACCGTGTCTAAGGGCGTGCAGATCAATA |
| | | TCAAGGCCATTCCATCTCCACCTACCGAGGTGTCCATCCGGAAT |
| | | AGCACAGCCCACTCCATCCTGATCTCTTGGGTGCCCGGCTTCGAT |
| | | GGCTACAGCCCCTTCAGAAACTGCTCCATCCAAGTGAAAGAGGC |
| | | CGATCCTCTGAGCAACGGCTCCGTGATGATCTTCAACACAAGCG |
| | | CCCTGCCACACCTGTACCAGATCAAACAGCTGCAGGCTCTGGCC |
| | | AACTACTCCATCGGCGTGTCCTGCATGAACGAGATCGGCTGGAG |
| | | TGCCGTGTCTCCTTGGATCCTGGCCAGCACAACTGAAGGCGCTC |
| | | CATCTGTGGCCCCTCTGAATGTGACCGTGTTCCTGAACGAGAGC |
| | | AGCGACAATGTGGACATCCGGTGGATGAAGCCACCTACAAAGC |
| | | AGCAGGACGGCGAACTCGTGGGCTACAGAATCTCTCACGTGTGG |
| | | CAGTCTGCCGGCATCTCCAAAGAACTCCTCGAAGAAGTGGGCCA |
| | | GAACGGCAGCAGAGCCAGGATCTCTGTGCAGGTCCACAACGCCA |
| | | CATGCACAGTGCGGATTGCCGCTGTGACAAGAGGCGGAGTGGGC |
| | | CCTTTTAGCGACCCCGTGAAGATCTTTATCCCCGCTCACGGCTGG |
| | | GTCGACTACGCCCCATCTTCTACACCAGCTCCAGGCAACGCTGA |
| | | CCCCGTGCTGATCATCTTCGGCTGCTTTTGCGGCTTTATCCTGAT |
| | | CGGCCTGATCCTGTACATCAGCCTGGCCATCAGAAAGCGGGTGC |
| | | AAGAGACAAAGTTCGGCAACGCCTTCACCGAAGAGGACAGCGA |
| | | GCTGGTGGTCAACTATATCGCCAAGAAGTCCTTCTGCAGACGGG |
| | | CCATCGAGCTGACACTGCACAGTCTGGGAGTGTCCGAGGAACTG |
| | | CAGAACAAGCTGGAAGATGTGGTCATCGACCGGAACCTGCTGAT |
| | | CCTGGGCAAGATTCTCGGCGAGGGCGAGTTTGGCTCTGTGATGG |
| | | AAGGCAACCTGAAGCAAGAGGACGGCACCTCTCTGAAGGTGGC |
| | | CGTGAAAACCATGAAGCTGGACAACAGCAGCCAGCGCGAGATC |
| | | GAAGAGTTTCTGTCTGAGGCCGCCTGTATGAAGGATTTCTCTCAC |
| | | CCCAACGTGATCCGGCTGCTGGGCGTGTGTATCGAGATGTCTAG |
| | | CCAGGGCATCCCCAAGCCTATGGTCATCCTGCCTTTCATGAAGTA |
| | | CGGCGATCTGCACACCTACCTGCTGTACTCCAGACTGGAAACAG |
| | | GCCCCAAGCACATCCCTCTGCAGACCCTGCTGAAGTTCATGGTG |
| | | GATATCGCCCTCGGCATGGAATACCTGAGCAACCGGAACTTCCT |
| | | GCACCGCGATCTGGCCGCCAGAAATTGCATGCTGAGGGACGACA |
| | | TGACCGTGTGCGTGGCCGATTTTGGCCTGAGCAAGAAGATCTAC |
| | | AGCGGCGACTACTACCGGCAGGGCAGAATTGCCAAGATGCCCGT |
| | | GAAGTGGATCGCCATCGAGAGCCTGGCCGACAGAGTGTACACCA |
| | | GCAAGTCTGACGTGTGGGCCTTCGGCGTGACCATGTGGGAGATT |
| | | GCCACCAGAGGCATGACCCCTTATCCTGGCGTCCAGAACCACGA |
| | | GATGTACGATTACCTGCTGCACGGCCACAGACTGAAGCAGCCAG |
| | | AGGATTGCCTGGACGAGCTGTACGAGATCATGTACTCTTGCTGG |
| | | CGGACCGATCCACTGGACAGACCTACATTCTCCGTGCTGCGGCT |
| | | GCAGCTGGAAAAACTGCTGGAAAGCCTGCCTGACGTGCGGAACC |
| | | AGGCCGATGTGATCTACGTGAACACCCAGCTGCTGGAATCCAGC |
| | | GAAGGACTGGCCCAGGGATCTACACTGGCTCCTCTGGACCTGAA |
| | | CATCGACCCCGACAGCATTATCGCCAGCTGCACACCAAGAGCCG |
| | | CCATCAGCGTTGTGACAGCCGAGGTGCACGATAGCAAGCCTCAC |
| | | GAAGGCCGGTACATCCTGAATGGCGGAAGCGAGGAATGGGAAG |
| | | ATCTGACCAGCGCTCCTTCTGCCGCCGTGACCGCCGAGAAAAAT |
| | | TCTGTGCTGCCTGGCGAGCGGCTCGTGCGAAACGGTGTCTCTTG |
| | | GAGCCACAGCTCCATGCTGCCTCTGGGAAGCTCTCTGCCTGATG |
| | | AGCTGCTGTTCGCCGACGATTCTAGCGAGGGATCCGAGGTGCTG |
| | | ATGTGA |
| 6 | MERTK-4 | ATGGGACCAGCGCCATTGCCTTTGTTGCTCGGGTTATTTCTGCCA |
| | | GCCCTGTGGCGGCGTGCCATTACAGAAGCTCGAGAGGAAGCTAA |
| | | ACCCTATCCATTGTTTCCAGGTCCCTTCCCTGGCTCATTGCAGAC |
| | | AGATCATACGCCTCTCTTGTCATTACCACATGCTAGCGGCTATCA |
| | | ACCAGCTCTCATGTTCTCTCCTACACAACCCGGCCGGCCCCACAC |
| | | CGGTAATGTGGCGATCCCACAAGTCACTAGTGTAGAGAGCAAAC |
| | | CACTGCCCCCTCTGGCTTTTAAGCATACTGTCGGGCATATCATCC |
| | | TGAGTGAGCACAAGGGAGTTAAGTTCAACTGTAGTATTTCCGTC |
| | | CCCAACATTTATCAAGATACGACCATATCCTGGTGGAAGGACGG |
| | | TAAAGAGCTGTTGGGCGCTCACCACGCCATCACCCAATTCTACC |
| | | CTGACGACGAGGTGACTGCCATCATAGCATCCTTTTCAATCACG |
| | | AGCGTACAAAGGTCCGATAACGGCTCTTACATATGCAAAATGAA |
| | | GATTAATAACGAGGAAATTGTTAGTGACCCGATTTATATTGAGG |
| | | TGCAGGGCCTGCCCCATTTCACAAAACAACCGGAATCCATGAAC |
| | | GTAACTCGAAATACCGCTTTTAATTTGACTTGCCAAGCCGTTGGA |
| | | CCCCCAGAACCGGTAAATATCTTTTGGGTGCAGAATAGCTCACG |
| | | AGTAAATGAGCAACCCGAGAAGTCTCCGTCTGTTTTAACAGTCC |
| | | CTGGGCTTACAGAAATGGCAGTGTTTTCTTGCGAAGCGCATAAC |
| | | GATAAGGGTTTGACAGTCAGCAAAGGCGTACAAATTAATATAAA |
| | | GGCCATCCCATCACCACCCACAGAGGTTTCAATACGCAATTCAA |
| | | CAGCCCATTCAATCCTCATTTCTTGGGTGCCCGGATTCGACGGGT |
| | | ATAGCCCTTTTAGAAACTGTTCAATCCAAGTGAAAGAGGCCGAC |
| | | CCTTTATCCAACGGGAGCGTGATGATCTTCAATACTTCAGCACTC |
| | | CCTCACCTTTATCAGATTAAACAACTTCAGGCGTTGGCCAACTAT |
| | | TCCATCGGGGTGAGTTGTATGAACGAGATTGGGTGGAGCGCTGT |
| | | ATCTCCATGGATCCTTGCTAGTACTACTGAGGGTGCACCCAGCGT |
| | | GGCTCCCTTGAACGTGACCGTCTTCCTCAACGAGTCATCCGACA |
| | | ACGTCGATATTCGATGGATGAAACCACCCACCAAACAACAAGAC |
| | | GGTGAGTTAGTCGGATATAGAATTAGTCATGTTTGGCAAAGCGC |
| | | TGGTATCAGTAAGGAATTGCTGGAAGAGGTCGGACAAAACGGA |
| | | AGTCGCGCCAGGATAAGTGTGCAGGTGCATAACGCCACTTGTAC |
| | | TGTAAGAATCGCTGCTGTGACACGAGGCGGCGTCGGCCCGTTTA |
| | | GCGACCCTGTAAAGATTTTCATTCCAGCGCATGGGTGGGTGGAC |
| | | TACGCTCCTAGCTCTACCCCTGCCCCCGGGAATGCTGACCCGGTC |
| | | CTTATTATTTTCGGATGTTTCTGCGGGTTCATCCTGATAGGTCTC |
| | | ATATTGTATATTTCTCTGGCAATTCGTAAGCGCGTGCAAGAAACC |
| | | AAATTCGGTAACGCCTTTACTGAAGAAGACAGCGAGCTGGTCGT |
| | | CAACTACATCGCTAAGAAGAGCTTTTGCAGGAGAGCTATAGAGC |
| | | TGACACTTCACTCACTGGGTGTTAGCGAAGAGCTGCAGAACAAG |
| | | CTGGAAGACGTGGTCATCGATCGAAACCTGCTCATACTGGGCAA |
| | | GATCTTGGGCGAAGGGGAATTCGGTTCCGTGATGGAAGGCAACC |
| | | TGAAACAAGAGGACGGCACTAGCCTCAAGGTTGCCGTCAAAAC |
| | | AATGAAACTCGATAATAGTAGTCAAAGAGAAATTGAAGAATTCC |
| | | TGTCTGAAGCCGCCTGTATGAAGGATTTCTCCCATCCGAACGTG |
| | | ATCAGGCTCTTGGGCGTCTGCATTGAGATGTCTAGCCAGGGGAT |
| | | TCCTAAACCTATGGTTATCCTGCCATTTATGAAGTATGGTGATTT |
| | | GCACACATATCTCTTATACAGCCGCCTCGAAACCGGCCCCAAAC |
| | | ACATCCCATTGCAAACTCTTCTGAAATTTATGGTCGACATCGCTT |
| | | TAGGGATGGAATACTTGAGTAATCGAAACTTCCTGCACCGGGAC |
| | | CTGGCGGCCAGAAATTGTATGCTGAGAGACGATATGACCGTGTG |
| | | CGTGGCCGATTTTGGATTGTCAAAGAAAATCTATAGCGGTGACT |
| | | ACTATAGGCAGGGGAGAATCGCCAAAATGCCCGTGAAGTGGATT |
| | | GCAATCGAGAGCCTGGCGGATAGAGTGTATACTTCAAAGTCCGA |
| | | CGTCTGGGCCTTCGGTGTCACTATGTGGGAGATCGCGACTCGAG |
| | | GGATGACCCCGTACCCCGGAGTTCAAAATCACGAAATGTACGAT |
| | | TACTTACTTCACGGGCATCGCCTCAAACAACCAGAGGATTGTCTT |
| | | GACGAGCTCTACGAGATCATGTATAGTTGTTGGCGCACAGACCC |
| | | TCTGGATCGGCCTACATTCTCTGTGCTCCGCTTACAATTGGAGAA |
| | | GTTGCTCGAGTCTCTCCCAGATGTGAGAAATCAGGCTGATGTGA |
| | | TCTATGTGAACACCCAACTGCTTGAATCCAGCGAAGGACTTGCG |
| | | CAAGGGTCTACACTCGCCCCCTTAGATCTCAATATTGATCCAGAT |
| | | TCAATCATCGCGAGCTGTACCCCAAGAGCGGCAATTAGCGTTGT |
| | | GACCGCCGAGGTGCACGATTCCAAGCCCCACGAGGGGCGCTATA |
| | | TTTTAAACGGCGGATCTGAAGAGTGGGAAGACTTAACAAGTGCA |
| | | CCTAGCGCAGCTGTAACTGCCGAGAAGAATTCTGTACTTCCCGG |
| | | TGAACGCCTGGTGCGGAACGGAGTGAGTTGGTCACACAGCTCCA |
| | | TGCTCCCACTGGGTTCAAGTCTTCCGGACGAGCTCCTGTTCGCAG |
| | | ATGATTCTTCCGAGGGATCCGAGGTGCTTATGTAG |

In some aspects, provided herein are functional MERTK nucleic acid sequences comprising nucleic acid sequence at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identical to any one of SEQ ID NOs: 3-6. In some embodiments, the functional nucleic acid sequence comprises nucleic acid sequence at least 90%, 91%, 92%, 93%, 94%, 95%, 96%, 97%, 98%, 99% or more identical to SEQ ID NO: 3. In some embodiments, the functional nucleic acid sequence comprises nucleic acid sequence at least 90%, 91%, 92%, 93%, 94%, 95%, 96%, 97%, 98%, 99% or more identical to SEQ ID NO: 4. In some embodiments, the functional nucleic acid sequence comprises nucleic acid sequence at least 90%, 91%, 92%, 93%, 94%, 95%, 96%, 97%, 98%, 99% or more identical to SEQ ID NO: 5. In some embodiments, the functional nucleic acid sequence comprises nucleic acid sequence at least 90%, 91%, 92%, 93%, 94%, 95%, 96%, 97%, 98%, 99% or more identical to SEQ ID NO: 6.

A human MERTK protein can include one or more mutations, e.g., mutations at up to about 1%, at up to about 2%, at up to about 3%, at up to about 4%, at up to about 5%, at up to about 10%, at up to about 15%, at up to about 20%, or more of the residues. Such variants can retain the activity of the wild type protein. In some embodiments, the mutation is a conservative substitution. Such changes can include but are not limited to substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine(S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some cases, the length of a reference sequence aligned for comparison purposes is at least about 70%, at least about 75%, at least about 80%, at least about 82%, at least about 84%, at least about 86%, at least about 88%, at least about 90%, at least about 91%, at least about 92%, at least about 93%, at least about 94%, at least about 95%, at least about 96%, at least about 97%, at least about 98%, at least about 99%, or at least about 100% of the length of the reference sequence.

The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at geg.com), using the default parameters, e.g., a Blossum 62 scoring matrix with a gap penalty of 12, a gap extend penalty of 4, and a frameshift gap penalty of 5.

### Vectors

Described herein are targeted expression vectors for delivery, transfection, and expression of a polynucleotide that encodes a MERTK polypeptide as described herein. In some embodiments, the expression is targeted to the retinal cells (e.g., inner retinal cells or retinal pigment epithelium (RPE) cells). In some embodiments, the expression is targeted to photoreceptor cells (e.g., rod cells or cone cells). Expression constructs of such components can be administered in any effective carrier, e.g., any formulation or composition capable of effectively delivering the component gene to cells.

One approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Viral vectors can be used as a recombinant gene delivery system for the transfer of exogenous genes in vivo, particularly into humans. These vectors provide efficient delivery of genes into cells, and in some cases the transferred nucleic acids are stably integrated into the chromosomal DNA of the host. Protocols for producing recombinant viruses and for infecting cells in vitro or in vivo with such viruses can be found in Ausubel, et al., eds., Gene Therapy Protocols Volume 1: Production and In Vivo Applications of Gene Transfer Vectors, Humana Press, (2008), pp. 1-32 and other standard laboratory manuals.

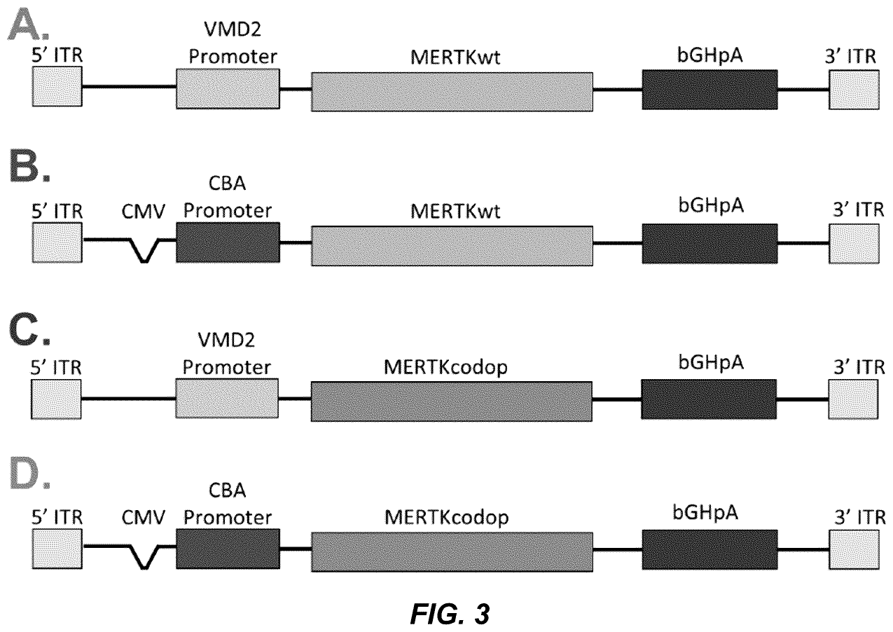

A viral vector can include, but is not limited to, recombinant retroviruses, adenovirus, adeno-associated virus (AAV), lentivirus, herpes simplex virus-1, alphavirus, vaccinia virus, or recombinant bacterial or eukaryotic plasmids. A vector can be a single-stranded vector or a double-stranded vector. A vector can be self-complementary. A vector can be linear. Alternatively, a vector can be circularized. In some cases, a vector can be a plasmid. In some cases, a vector can be the vector of FIG. 1B, pAAV.VMD2.hMERTK.bGH/SV40. In some embodiments, a vector can comprise any one of the vectors of FIG. 3. In some embodiments, the vector can comprise, from 5′ end to 3′ end: a 5′ ITR, a CMV enhancer, a CBA promoter, a codon-optimized MERTK coding nucleic acid sequence, a bovine growth hormone polyadenylation signal (bGHpA), and a 3′ ITR. In some embodiments, the vector can comprise, from 5′ end to 3′ end: a 5′ ITR, a CMV enhancer, a CBA promoter, a wildtype MERTK coding nucleic acid sequence, a bovine growth hormone polyadenylation signal (bGHpA), and a 3′ ITR. In some embodiments, the vector can comprise, from 5′ end to 3′ end: a 5′ ITR, a VMD2 promoter, a codon-optimized MERTK coding nucleic acid sequence, a bovine growth hormone polyadenylation signal (bGHpA), and a 3′ ITR. In some embodiments, the vector can comprise, from 5′ end to 3′ end: a 5′ ITR, a VMD2 promoter, a wildtype MERTK coding nucleic acid sequence, a bovine growth hormone polyadenylation signal (bGHpA), and a 3′ ITR. In some embodiments, the vector comprises a bGH polyA signal and does not comprise a SV40 poly A region.

Viral vectors can include adeno-associated viral vectors (AAV vectors). Adeno-associated virus is a naturally occurring defective virus that requires another virus, such as an adenovirus or a herpes virus, as a helper virus for efficient replication and a productive life cycle. AAV vectors efficiently transduce various cell types and can produce long-term expression of transgenes in vivo. Although AAV vector genomes can persist within cells as episomes, vector integration has been observed. AAV vectors, particularly AAV2, have been extensively used for gene augmentation or replacement and have shown therapeutic efficacy in a range of animal models as well as in the clinic. AAV vectors containing as little as 300 base pairs of AAV can be packaged and can produce recombinant protein expression. Space for exogenous DNA is limited to about 4.5 kb. An AAV can be a variant, or serotype of an AAV. An AAV serotype can be, but is not limited to, AAV1, AAV2, AAV3, AAV4, AAV5, AAV6, AAV7, AAV8, AAV9, AAV10, AAV11, AAV12, or AAV13. A variant of an AAV can comprise a genetically engineered AAV (e.g., AAV2.7m8 or AAV2tYF). A vector may comprise all of an AAV. Alternatively, or in addition to, a vector may comprise a portion of an AAV (e.g., an AAV capsid). A vector may comprise inclusions of amino acids from one AAV serotype into another AAV serotype (e.g., AAV2/5). An AAV vector can be a recombinant AAV (rAAV) vector.

A vector may comprise an AAV capsid protein. An AAV capsid protein is the protein shell enclosure produced by an AAV. An AAV capsid protein comprise viral proteins. An AAV capsid protein can comprise at least 1, at least 2, at least 3, at least 4, at least 5, or more viral proteins. An AAV capsid can be, but is not limited to, an AAV1 capsid protein, an AAV2 capsid protein, an AAV2tYF capsid protein, an AAV2.7m8 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, or an AAV13 capsid protein.

An AAV may be specialized for use in an ocular environment (e.g., AAV2, AAV4, AAV5, or AAV8). An AAV vector can be used to introduce DNA into the retina, e.g., into photoreceptors (e.g., rod cells or cone cells), inner retinal cells, or retinal pigment epithelium (RPE) cells.

Viral vectors can transfect cells directly; plasmid DNA can be delivered naked or with the help of, for example, cationic liposomes (lipofectamine) or derivatized (e.g., antibody conjugated), cationic dendrimers, inorganic vectors (e.g., iron oxide magnetofection), lipidoids, lipid nanoparticles (LNPs) (e.g., solid lipid nanoparticles, polymer lipid nanoparticles), cell-penetrating peptides, pamam dendrimers, phosphorylcholine (e.g., methylphosphorylcholine), cyclodextrin polymer (CDP), polylysine conjugates, gramacidin S, artificial viral envelopes or other such intracellular carriers. Alternatively, plasmid DNA can be delivered through direct injection of the gene construct, electroporation, or using CaPO₄ precipitation.

LNPs can be used to deliver an AAV vector. An LNP can comprise a cationic lipid, a sterol, a neutral lipid, and/or a polyethylene glycol (PEG). An LNP can have size of at least about 5 nm, at least about 10 nm, at least about 15 nm, at least about 20 nm, at least about 30 nm, at least about 40 nm, at least about 50 nm, at least about 60 nm, at least about 70 nm, at least about 80 nm, at least about 90 nm, at least about 100 nm, at least about 110 nm, at least about 120 nm, at least about 130 nm, at least about 140 nm, at least about 150 nm, at least about 160 nm, at least about 170 nm, at least about 180 nm, at least about 190 nm, at least about 200 nm, at least about 250 nm, at least about 300 nm, or greater. An LNP can have a size of at most about 300 nm, at most about 250 nm, at most about 200 nm, at most about 190 nm, at most about 180 nm, at most about 170 nm, at most about 160 nm, at most about 150 nm, at most about 140 nm, at most about 130 nm, at most about 120 nm, at most about 110 nm, at most about 100 nm, at most about 90 nm, at most about 80 nm, at most about 70 nm, at most about 60 nm, at most about 50 nm, at most about 40 nm, at most about 30 nm, at most about 20 nm, at most about 15 nm, at most about 10 nm, at most about 5 nm, or less. In some cases, the AAV vector or LNP is in a liquid (e.g., an emulsion or a solution). In some cases, LNP is a solid LNP or a polymer LNP. A polymer LNP can be but is not limited to poly-lactic-co glycolic acid (PLGA) nanoparticles or poly-β-amino-ester (PBAE) nanoparticles. In some cases, the LNPs are sorted to obtain an LNP population of approximately the same size. Alternatively, the LNPs can be different sizes.

In some embodiments, the AAV vector can comprise one or more helper elements derived from helper viruses such as Adenoviruses (Ad), herpes simplex virus (HSV), or human papillomavirus (HPV). In some cases, the one or more helper elements comprise EIA, EIB, VA, E2A DNA-binding protein (DBP) or E4 region from an adenovirus. In some cases, the AAV vector comprises the E4 gene. In some cases, the AAV vector comprises the E2a gene. In some cases, the AAV vector comprises the VA gene. In some cases, the AAV vector comprises two open reading frames. In some cases, the AAV vector comprises at least one, at least two, at least three, at least four, at least five, at least six, or more open reading frame. In some cases, the AAV vector comprises at most six, at most five, at most four, at most three, at most two, or at most one open reading frame(s).

In some cases, the AAV comprises a non-coding sequence or a regulatory sequence that can modulate the expression of the gene of interest. The regulatory sequence can be derived from a eukaryotic genome or a non-eukaryotic genome (e.g., a viral or a bacterial genome). The non-coding sequence or regulatory sequence can comprise a 5′ untranslated region (5′-UTR), a 3′ untranslated region (3′-UTR), a promoter, an enhancer, a polyadenylation (poly-A) sequence, or a cis-regulatory element. In some embodiments, the cis-regulatory element comprises a silencer, an operator, a woodchuck hepatitis virus post-transcriptional regulatory element (WPRE), or a minute virus of mice (MVM) intron. In some embodiments, the non-coding sequence or regulatory sequence can comprise a portion of an exon or a portion of an intron. In some embodiments, the regulatory sequence comprises a polyadenylation sequence (poly A). The poly A sequence can comprise a bovine growth hormone polyadenylation sequence (e.g., bGH poly A) or a SV40 poly A sequence. In some embodiments, the poly A sequence comprises a bGH poly A and a SV40 poly A sequence. In some embodiments, the poly A sequence comprises a bGH poly A and does not comprise a SV40 poly A sequence. In some embodiments, the regulatory sequence comprises a Kozak sequence.

In some cases, the AAV comprises a promoter sequence. In some cases, the promoter can include, but is not limited to, a Cytomegalovirus (CMV) promoter, a hybrid CMV enhancer/chicken β-acting (CBA) promoter, a CAG promoter, a rhodopsin (RHO) promoter (e.g., a human rhodopsin (hRHO) promoter, a Rho194 promoter), a CASI promoter, a rhodopsin kinase promoter (e.g., a GRK1 promoter), an interphotoreceptor retinoid binding protein (IRBP) promoter, a red opsin promoter, a vitelliform macular dystrophy (VMD2) promoter, and a cadherin promoter (e.g., a CDH5 promoter or a CD144 promoter). Vectors can include promoters that drive expression in many cell types (e.g., CAG, CMV, or CASI). Alternatively, vectors can include promoters that drive expression in specific cell types, such as photoreceptor cells (RHO, rhodopsin kinase (GRK1), cone arrestin (CAR)) or RPE cells (e.g., promotors for RPE-specific proteins such as VMD2, RPE65, RLBP1, RGR, or TIMP3). Synthetic promoters ProC1 and ProD5 can also be used.

In some cases, the AAV comprises an inverted terminal repeat (ITR) sequence. An ITR sequence can be on the 3′ end of a vector. Alternatively or in addition to, an ITR sequence can be on the 5′ end of a vector. An ITR can fold to make double-stranded DNA from single-stranded DNA.

In some cases, the AAV comprises an enhancer. An enhancer can include, but is not limited to a CMV enhancer, a CBA/CAG enhancer, a ubiquitin enhancer, a RHO enhancer, a rhodopsin kinase enhancer, an IRBP enhancer, a red opsin enhancer, a VMD2 enhancer, a CASI enhancer, and a cadherin enhancer. Alternatively, or in addition to, a AAV can comprise another type of cis-regulatory element (e.g., a silencer, an operator, a woodchuck hepatitis virus post-transcriptional regulatory element (WPRE), or a minute virus of mice (MVM) intron).

In some cases, the AAV comprises an expression cassette. An expression cassette can comprise a recombinant DNA molecule containing a desired coding sequence for a gene of interest (e.g., MERTK) and appropriate nucleic acid sequence necessary for the expression of the operably linked coding sequence in a particular host organism. An expression cassette can comprise at least 1, at least 2, at least 3, at least 4, at least 5, at least 6, at least 7, at least 8, at least 9, at least 10, or more gene(s). An expression cassette can comprise at most 10, at most 9, at most 8, at most 7, at most 6, at most 5, at most 4, at most 3, at most 2, or at most 1 gene(s). In some embodiments, the vector provided herein comprises an AAV expression cassette comprising a MERTK coding sequence.

In some embodiments, a gene encoding a MERTK is entrapped in liposomes bearing positive charges on their surface (e.g., lipofectins), which can be tagged with antibodies against cell surface antigens of the target tissue. In some cases, a gene encoding a MERTK is entrapped in liposomes bearing negative charges on their surface. In some cases, a gene encoding a MERTK is entrapped in liposomes bearing zwitterionic charges on their surface. In some cases, a gene encoding a MERTK is entrapped in liposomes bearing no ionic moieties on their surface.

The pharmaceutical preparation of the gene therapy construct can consist essentially of the gene delivery system (viral vector and any associated agents such as helper viruses, proteins, lipids, and so on) in an acceptable diluent, or can comprise a slow-release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

A vector can comprise a pharmaceutically acceptable carrier. A pharmaceutically acceptable carrier can be, but is not limited to, an excipient, an adhesive, a disintegrant, a lubricant, a stabilizer, a diluent, a solvent for injection, an oleaginous base, a sense of touch modifier, a surfactant, a macromolecule, a thick-gelling agent, a solvent, a propellant, an antioxidant, a reducing agent, an oxidant, a chelating agent, an acid, an alkali, powder, inorganic salts, water, an unsaturated monomer, a polyalcohol, a polymeric additive, an auxiliary agent, a wetting agent, a thickener, or a thickening material.

In some aspects, the present disclosure provides a composition comprising any one of the nucleic acids and/or nucleic acid sequences encoding a human MERTK provided herein. The composition can comprise any one of the functional MERTK nucleic acid sequences provided herein. In some embodiments, the composition comprises a functional MERTK nucleic acid sequence at least 90%, 95%, or 99% identical to any one of SEQ ID NOs: 3-6. The composition can further comprise a non-coding nucleic acid sequence and/or a regulatory nucleic acid sequence provided herein. In some embodiments, the composition further comprises a promoter which expresses a product of the functional MERTK nucleic acid sequence in a plurality of photoreceptor cells or retinal pigment epithelium cells. In some embodiments, the composition further comprises a pharmaceutically acceptable carrier.

In some aspects, the present disclosure provides a composition comprising any one of the vectors provided herein. In some embodiments, the composition comprises a recombinant AAV (rAAV) vector provided herein. In some embodiments, the composition comprises a rAAV selected from the group consisting of AAV2, AAV5, AAV8, AAV9, AAV2/5, AAV 2tYF, and AAV2.7m8. In some embodiments, the rAAV is a rAAV2. In some embodiments, the rAAV is a rAAV5. In some embodiments, the rAAV is a rAAV4.

### Therapeutic Methods

MERTK can be essential for the maintenance of proper vision. Gene therapy and other methods disclosed herein can be used for the correction of ocular diseases and disorders. Ocular diseases and disorders can include, but are not limited to uveitis, glaucoma, macular edema, diabetic macular edema, retinopathy, age-related macular degeneration (e.g., wet AMD or dry AMD), scleritis, optic nerve degeneration, geographic atrophy, macular dystrophy, choroidal disease, ocular sarcoidosis, optic neuritis, choroidal neovascularization, ocular cancer, genetic disease(s), autoimmune diseases affecting the posterior segment of the eye, retinitis (e.g., cytomegalovirus retinitis, retinitis pigmentosa), and corneal ulcers. In some embodiments, the ocular disease or disorder comprises a MERTK-mediated disease or disorder. In some embodiments, the MERTK-mediated disease or disorder comprises retinitis pigmentosa.

In some embodiments herein, a gene therapy can treat retinitis pigmentosa. Retinitis pigmentosa (RP) is a group of rare eye diseases (e.g., inherited retinal dystrophies) that affect the retina (the light-sensitive layer of tissue in the back of the eye). RP makes cells in the retina break down slowly over time, causing vision loss, and can be characterized by abnormalities of the photoreceptors (rods and/or cones) of the retinal pigment epithelium (RPE). Early symptoms of RP are loss of night vision and loss of peripheral vision. Other symptoms of RP can include but are not limited to reduced visual field, night blindness, attenuation of retinal vessels, optic disc pallor, auto-fluorescent macula, macular atrophy, and bone spicule pigments. Retinitis pigmentosa is a progressive disease that can eventually cause partial or total blindness. In some embodiments herein, retinitis pigmentosa is autosomal recessive retinitis pigmentosa. In some embodiments herein, retinitis pigmentosa is autosomal dominant retinitis pigmentosa. In some embodiments herein, retinitis pigmentosa is retinitis pigmentosa 38 (RP38). In some embodiments herein, retinitis pigmentosa is a rod-cone dystrophy.

A therapeutic method can comprise the administration of a MERTK gene (e.g., a wildtype gene or a codon-optimized gene) to a subject. The therapeutic method can comprise the administration of a composition provided herein to a subject. In some embodiments, the therapeutic method comprises the administration of a nucleic acid sequence encoding the MERTK gene (e.g., a wildtype gene or a codon-optimized gene) to a subject. In some embodiments, the therapeutic method comprises the administration of a rAAV comprising the MERTK gene. Administration can be topical (e.g., eye drops, ointments), oral (e.g., tablets, capsules, liquids), or through injection. Injection of a therapy into the eye can be intravitreal, subretinal, or suprachoroidal. In some embodiments, administering a MERTK gene to a subject occurs prior to onset of the ocular diseases and disorders. In some embodiments, administering a MERTK gene to a subject occurs after onset of the ocular diseases and disorders.

A gene therapy can be administered in an amount of at least about 10⁷, at least about 10⁸, at least about 10⁹, at least about 10¹⁰, at least about 10¹¹, at least about 10¹², or at least about 10¹³ viral particles per mL. A gene therapy can be administered in an amount of at most about 10¹³, at most about 10¹², at most about 10¹¹, at most about 10¹⁰, at most about 10⁹, at most about 10⁸, or at most about 10⁷ viral particles per mL. A gene therapy can be administered to one eye of a subject. Alternatively, a gene therapy can be administered to both eyes of a subject. In some embodiments, the MERTK gene administering is performed at an amount of at least 10⁹ viral particles per mL. In some embodiments, the MERTK gene administering is performed at an amount of at least 10¹⁰ viral particles per mL.

Administration of a therapy can improve vision loss. In some cases, administration of a therapy can restore at least partial vision to a subject. In some cases, administration of a therapy can fully restore vision to a subject. Administration of a therapy can improve vision loss by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more, when measured using a visual field test. Administration of a therapy can improve vision loss by at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 19%, at most about 18%, at most about 17%, at most about 16%, at most about 15%, at most about 14%, at most about 13%, at most about 12%, at most about 11%, at most about 10%, at most about 9%, at most about 8%, at most about 7%, at most about 6%, at most about 5%, at most about 4%, at most about 3%, at most about 2%, at most about 1%, or less, when measured using a visual field test.

Administration of a therapy can improve phagocytosis activity of retinal pigment epithelium (RPE). The ability of RPE to uptake photoreceptor outer segments (POS) can be measured via phagocytosis assays. For example, POS can be labeled with a fluorescence dye and RPE phagocytosis of labeled POS can be measured by fluorescence microscopy or flow cytometry. In some embodiments, administration of a therapy increases internalization of POS by RPE. Administration of a therapy can increase POS internalization by RPE by at least about 10%, at least about 20%, at least about 30%, at least about 40%, at least about 50%, at least about 60%, at least about 70%, at least about 80%, at least about 90%, at least about 100%, at least about 110%, at least about 120%, at least 130%, at least about 140%, at least about 150%, at least about 160%, at least about 170%, at least about 180%, at least about 190%, at least about 200%, at least about 200%, at least about 210%, at least about 220%, at least 230%, at least about 240%, at least about 250%, at least about 260%, at least about 270%, at least about 280%, at least about 290%, at least 300%, at least 350%, at least 400%, at least 450%, at least 500%, at least 600%, at least 700%, at least 800%, at least 900%, or more, when measured using a phagocytosis assay.

A subject can be an animal such as a monkey, an ape (e.g., a chimpanzee, a gorilla), a horse, a cow, a pig, a sheep, a goat, a dog, a cat, a rabbit, a guinea pig, a gerbil, a hamster, a rat, a mouse, a camillid, a bird, a reptile, or an amphibian. A subject can be a mammal. A subject can be a human. A subject can be a human that has an ocular disease or disorder. Alternatively, or in addition to, a subject can be a human that is genetically susceptible or genetically predisposed to an ocular disease or disorder.

Another aspect provides a kit that comprises the compositions comprising a MERTK coding nucleic acid sequence provided herein. In some embodiments, the kit further comprises a second therapeutic agent. The second therapeutic agent can be a small molecule drug, a protein, a nucleic acid, a virus, or a combination there of. In some embodiments, the kit further comprises an apparatus for administering the compositions provided herein, such as appropriate tubing for administration to the eye. In some embodiments, the kit further comprises instructions for use (IFU). The IFU can include instructions for administering the composition provided herein to an eye of a subject.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: MERTK Gene Therapy in iPSC-RPE Cells

In this experiment, rAAV2-hMERTK constructs were designed and screened to validate their ability to restore MERTK expression and function in a MERTK-based RP patient-derived iPSC-RPE cell line. An exemplary gene construct is depicted in FIG. 1A, while an exemplary vector is shown in FIG. 1B.

### Methods

AAV-hMERTK plasmids were designed with either a hybrid cytomegalovirus chicken β-actin (CBA) promoter or an RPE-specific vitelliform macular dystrophy-2 (VMD2) promoter; and two different MERTK sequences (native, wt and codon optimized, codop) (FIG. 3). MERTK plasmids were generated on a pUC-Kanamyacin backbone containing 5′ and 3′ inverted terminal repeats (ITRs) and a bovine growth hormone polyadenylation signal (bGHpA) with either a variation in the chosen promoter or chosen gene of interest sequence. MERTK plasmid sequences can be found in Table 3.

MERTK expression was measured in multiple RPE cell models by digital PCR using MERTKwt primer sets listed in Table 4. Cell lines tested include ARPE19 cells (human spontaneously arising RPE cells), ARPE19 cells differentiated in MEM-Nicotinamide media for 1 and 2 weeks, JBWT11 p28 (induced Pluripotent stem cells (iPSC) (non-RPE cells)), iPSC-RPE cells (differentiated RPE cells from human iPSCs), and 293T cells (human embryonic kidney cells (non-RPE cells)).

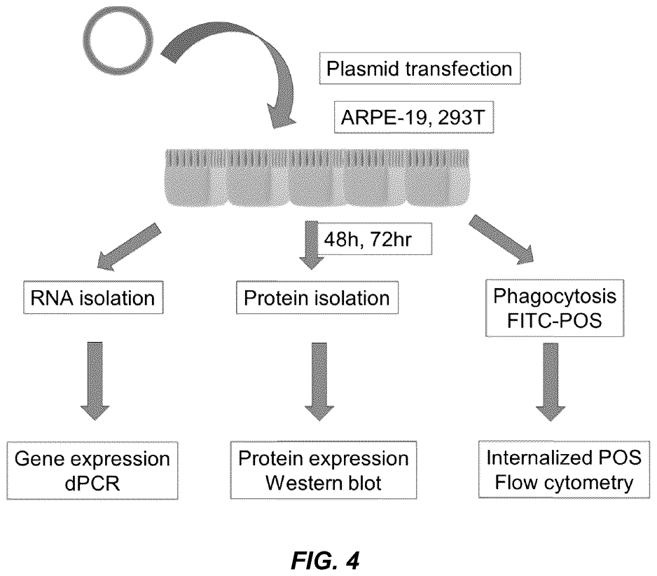

ARPE19, 293T, and iPSC-RPE (proband and parental) cells were transfected with four MERTK constructs: (VMD2-hMERTKwt, VMD2-hMERTKcodop, CBA-hMERTKwt and CBA-hMERTKcodop) and MERTK expression was quantified by digital PCR and western blot (FIG. 4). Cells were transfected using 500 ng plasmid per 400,000 cells using Lipofectamine™ reagent. Mock (Lipofectamine+nuclease free water) transfected cells were used as a control. Cells were collected for protein and RNA isolation after both 48 hours and 72 hours.

RPE cells were infected with MERTK constructs packaged in AAV2-serotype. MERTK expression (protein and RNA) and phagocytic activity were measured. The effect of MERTK mutation on global gene expression in iPSC-RPE cells (parental and proband) was also characterized by RNA-seq. Digital PCR was performed using a QIAcuity2-Plex system. MERTK primer sequences are shown in Table 4. For Western blots, SDS-PAGE was performed on a 10% gel using total cell lysates and the blot was probed with MERTK antibody. Transfected ARPE19 cells were treated with FITC-POS, cells were washed, trypsinized and cell lysates were collected at 30 mins, 60 mins and 120 mins. Blots were probed with rhodopsin antibody to study the time course of POS internalization. GAPDH was used as the loading control. For phagocytosis, the transfected ARPE19 cells were incubated with bPOS-FITC 10bPOS per cell) for three hours. Cells were washed, trypsinized, and stained with DRAQ5 before being assayed by flow cytometry to measure the percentage of DRAQ5+ and FITC+ cells, which represented the percentage of cells with internalized POS.

Viability of transfected cells was measured. The ARPE19 cells were treated with Cell Titer Glo 2.0 48 hours post transfection and luminescence was measured.

One-way ANOVA with Dunnett's test for multiple comparisons was performed; values were considered statistically significant at p<0.05.

ARPE19 cells were transduced with AAV2-VMD2-MERTKwt and AAV2-VMD2-MERTKcodop at multiplicities of infection (MOI) according to Table 2, for 48 hrs. The cells were washed, trypsinized and pelleted. RNA was isolated from the cells, followed by cDNA preparation. Digital PCR was performed (QIAcuity One Plex) with 10 ng cDNA input per well. Primers specific for native MERTK (MERTKwt) were used to detect MERTK expression in cells transduced with AAV2-VMD2-MERTKwt and primers specific for codon optimized MERTK (MERTKcodop) were used to detect MERTKcodop expression in cells transduced with AAV2-VMD2-MERTKcodop. Primers (VIC fluorophore) for housekeeping gene RPP30 (Taqman id: Hs01124518_m1) was used to normalize the MERTK expression in each reaction. Diluent was used as control. Relative expression of gene of interest (GOI, MERTK) was calculated using the following formula and plotted as function of MOI:

$$Relative GOI = ( copies / ng - MERTK ) / ( copies / ng - RPP 30 )$$

TABLE 2
MOI for transduction with AAV2-VMD2-MERTKwt and AAV2-VMD2-MERTKcodop

| | Vector Stock | | Desired | total vg | uL vector |
|---|---|---|---|---|---|
| | (vg/uL) | Cells/well | vg/cell MOI | needed | needed |
| AAV2-VMD2-MERTKwt | 1.81E+08 | 150000 | 1.00E+04 | 1.50E+09 | 8.3 |
| AAV2-VMD2-MERTKwt | 9.05E+08 | 150000 | 5.00E+04 | 7.50E+09 | 8.3 |
| AAV2-VMD2-MERTKwt | 1.81E+09 | 150000 | 1.00E+05 | 1.50E+10 | 8.3 |
| AAV2-VMD2-MERTKwt | 1.10E+10 | 150000 | 6.09E+05 | 9.13E+10 | 8.3 |
| AAV2-VMD2-MERTKcodop | 1.81E+08 | 150000 | 1.00E+04 | 1.50E+09 | 8.3 |
| AAV2-VMD2-MERTKcodop | 9.05E+08 | 150000 | 5.00E+04 | 7.50E+09 | 8.3 |
| AAV2-VMD2-MERTKcodop | 1.81E+09 | 150000 | 1.00E+05 | 1.50E+10 | 8.3 |
| AAV2-VMD2-MERTKcodop | 7.50E+09 | 150000 | 4.15E+05 | 6.23E+10 | 8.3 |
| | Diluent | 150000 | | | 8.3 |

TABLE 3
AAV-hMERTK Plasmids

| SEQ ID | Sequence | |
|---|---|---|
| NO | Name | Sequence |
| 7 | CBA- | TCGCGCGTTTCGGTGATGACGGTGAAAACCTCTGACACATGCA |
| | hMERTK- | GCTCCCGGAGACTGTCACAGCTTGTCTGTAAGCGGATGCCGG |
| | codop | GAGCAGACAAGCCCGTCAGGGCGCGTCAGCGGGTGTTGGCGG |
| | | GTGTCGGGGCTGGCTTAACTATGCGGCATCAGAGCAGATTGT |
| | | ACTGAGAGTGCACCATATGCGGTGTGAAATACCGCACAGATG |
| | | CGTAAGGAGAAAATACCGCATCAGGCGCCATTCGCCATTCAG |
| | | GCTGCGCAACTGTTGGGAAGGGCGATCGGTGCGGGCCTCTTC |
| | | GCTATTACGCCAGCTGGCGAAAGGGGGATGTGCTGCAAGGCG |
| | | ATTAAGTTGGGTAACGCCAGGGTTTTCCCAGTCACGACGTTGT |
| | | AAAACGACGGCCAGTGAATTGACGCGTATTGGGATGTTTAAA |
| | | Cctgcgcgctcgctcgctcactgaggccgcccgggcaaagcccgggcgtcgggcgacctttggtcg |
| | | cccggcctcagtgagcgagcgagcgcgcagagagggagtggccaactccatcactaggggttcctt |
| | | gtagttaatgattaacccgccatgctacttatctacgtagcgcggccgcggacattgattattgactagtta |
| | | ttaatagtaatcaattacggggtcattagttcatagcccatatatggagttccgcgttacataacttacggta |
| | | aatggcccgcctggctgaccgcccaacgacccccgcccattgacgtcaataatgacgtatgttcccata |
| | | gtaacgccaatagggactttccattgacgtcaatgggtggagtatttacggtaaactgcccacttggcag |
| | | tacatcaagtgtatcatatgccaagtacgccccctattgacgtcaatgacggtaaatggcccgcctggca |
| | | ttatgcccagtacatgaccttatgggactttcctacttggcagtacatctacgtattagtcatcgctattacc |
| | | atggtcgaggtgagccccacgttctgcttcactctccccatctcccccccctccccacccccaattttgta |
| | | tttatttattttttaattattttgtgcagcgatgggggcggggggggggggggggcgcgcgccaggcgg |
| | | ggcggggcggggcgaggggcggggcggggcgaggcggagaggtgcggcggcagccaatcaga |
| | | gcggcgcgctccgaaagtttccttttatggcgaggcggcggcggcggcggcccTATAAAaagc |
| | | gaagcgcgcggcgggcgggagtcGctGcGttgcgatcgcaggtaagtttagtctttttgtcttttatttc |
| | | aggtcccggatccggtggtggtgcaaatcaaagaactgctcctcagtggatgttgcctttacttctaggc |
| | | ctgcaggccgccaccATGGGACCTGCTCCTCTGCCTCTGCTGCTGGGA |
| | | CTGTTTCTGCCTGCTCTTTGGCGGAGAGCCATCACCGAGGCCA |
| | | GAGAGGAAGCCAAGCCTTATCCTCTGTTCCCCGGACCTTTTCC |
| | | AGGCAGCCTGCAGACCGATCACACCCCTTTGCTGTCTCTGCCT |
| | | CACGCCTCTGGCTATCAGCCCGCTCTGATGTTCAGCCCCACAC |
| | | AGCCAGGCAGACCTCACACAGGCAATGTGGCCATTCCTCAAG |
| | | TGACCAGCGTGGAAAGCAAGCCCTTGCCTCCTCTGGCCTTCAA |
| | | GCACACAGTGGGCCACATCATCCTGAGCGAGCACAAGGGCGT |
| | | GAAGTTCAACTGCAGCATCAGCGTGCCCAACATCTACCAGGA |
| | | CACCACCATCAGCTGGTGGAAGGACGGCAAAGAACTGCTGGG |
| | | AGCCCACCACGCCATCACACAGTTCTACCCCGACGATGAAGT |
| | | GACCGCCATCATTGCCAGCTTCAGCATCACCAGCGTGCAGAG |
| | | AAGCGACAACGGCAGCTACATCTGCAAGATGAAGATCAACAA |
| | | CGAGGAAATCGTCAGCGACCCCATCTACATCGAGGTGCAGGG |
| | | CCTGCCTCACTTCACCAAGCAGCCCGAGAGCATGAACGTGAC |
| | | CAGAAACACCGCCTTCAACCTGACCTGTCAGGCCGTGGGACC |
| | | TCCTGAGCCTGTGAACATCTTCTGGGTGCAGAACAGCTCCAGA |
| | | GTGAACGAGCAGCCTGAGAAGTCCCCTAGCGTGCTGACAGTG |
| | | CCTGGACTGACAGAGATGGCCGTGTTTTCTTGCGAGGCCCACA |
| | | ACGACAAGGGCCTGACCGTGTCTAAGGGCGTGCAGATCAATA |
| | | TCAAGGCCATTCCATCTCCACCTACCGAGGTGTCCATCCGGAA |
| | | TAGCACAGCCCACTCCATCCTGATCTCTTGGGTGCCCGGCTTC |
| | | GATGGCTACAGCCCCTTCAGAAACTGCTCCATCCAAGTGAAA |
| | | GAGGCCGATCCTCTGAGCAACGGCTCCGTGATGATCTTCAACA |
| | | CAAGCGCCCTGCCACACCTGTACCAGATCAAACAGCTGCAGG |
| | | CTCTGGCCAACTACTCCATCGGCGTGTCCTGCATGAACGAGAT |
| | | CGGCTGGAGTGCCGTGTCTCCTTGGATCCTGGCCAGCACAACT |
| | | GAAGGCGCTCCATCTGTGGCCCCTCTGAATGTGACCGTGTTCC |
| | | TGAACGAGAGCAGCGACAATGTGGACATCCGGTGGATGAAGC |
| | | CACCTACAAAGCAGCAGGACGGCGAACTCGTGGGCTACAGAA |
| | | TCTCTCACGTGTGGCAGTCTGCCGGCATCTCCAAAGAACTCCT |
| | | CGAAGAAGTGGGCCAGAACGGCAGCAGAGCCAGGATCTCTGT |
| | | GCAGGTCCACAACGCCACATGCACAGTGCGGATTGCCGCTGT |
| | | GACAAGAGGCGGAGTGGGCCCTTTTAGCGACCCCGTGAAGAT |
| | | CTTTATCCCCGCTCACGGCTGGGTCGACTACGCCCCATCTTCT |
| | | ACACCAGCTCCAGGCAACGCTGACCCCGTGCTGATCATCTTCG |
| | | GCTGCTTTTGCGGCTTTATCCTGATCGGCCTGATCCTGTACATC |
| | | AGCCTGGCCATCAGAAAGCGGGTGCAAGAGACAAAGTTCGGC |
| | | AACGCCTTCACCGAAGAGGACAGCGAGCTGGTGGTCAACTAT |
| | | ATCGCCAAGAAGTCCTTCTGCAGACGGGCCATCGAGCTGACA |
| | | CTGCACAGTCTGGGAGTGTCCGAGGAACTGCAGAACAAGCTG |
| | | GAAGATGTGGTCATCGACCGGAACCTGCTGATCCTGGGCAAG |
| | | ATTCTCGGCGAGGGCGAGTTTGGCTCTGTGATGGAAGGCAAC |
| | | CTGAAGCAAGAGGACGGCACCTCTCTGAAGGTGGCCGTGAAA |
| | | ACCATGAAGCTGGACAACAGCAGCCAGCGCGAGATCGAAGA |
| | | GTTTCTGTCTGAGGCCGCCTGTATGAAGGATTTCTCTCACCCC |
| | | AACGTGATCCGGCTGCTGGGCGTGTGTATCGAGATGTCTAGCC |
| | | AGGGCATCCCCAAGCCTATGGTCATCCTGCCTTTCATGAAGTA |
| | | CGGCGATCTGCACACCTACCTGCTGTACTCCAGACTGGAAACA |
| | | GGCCCCAAGCACATCCCTCTGCAGACCCTGCTGAAGTTCATGG |
| | | TGGATATCGCCCTCGGCATGGAATACCTGAGCAACCGGAACT |
| | | TCCTGCACCGCGATCTGGCCGCCAGAAATTGCATGCTGAGGG |
| | | ACGACATGACCGTGTGCGTGGCCGATTTTGGCCTGAGCAAGA |
| | | AGATCTACAGCGGCGACTACTACCGGCAGGGCAGAATTGCCA |
| | | AGATGCCCGTGAAGTGGATCGCCATCGAGAGCCTGGCCGACA |
| | | GAGTGTACACCAGCAAGTCTGACGTGTGGGCCTTCGGCGTGA |
| | | CCATGTGGGAGATTGCCACCAGAGGCATGACCCCTTATCCTGG |
| | | CGTCCAGAACCACGAGATGTACGATTACCTGCTGCACGGCCA |
| | | CAGACTGAAGCAGCCAGAGGATTGCCTGGACGAGCTGTACGA |
| | | GATCATGTACTCTTGCTGGCGGACCGATCCACTGGACAGACCT |
| | | ACATTCTCCGTGCTGCGGCTGCAGCTGGAAAAACTGCTGGAA |
| | | AGCCTGCCTGACGTGCGGAACCAGGCCGATGTGATCTACGTG |
| | | AACACCCAGCTGCTGGAATCCAGCGAAGGACTGGCCCAGGGA |
| | | TCTACACTGGCTCCTCTGGACCTGAACATCGACCCCGACAGCA |
| | | TTATCGCCAGCTGCACACCAAGAGCCGCCATCAGCGTTGTGAC |
| | | AGCCGAGGTGCACGATAGCAAGCCTCACGAAGGCCGGTACAT |
| | | CCTGAATGGCGGAAGCGAGGAATGGGAAGATCTGACCAGCGC |
| | | TCCTTCTGCCGCCGTGACCGCCGAGAAAAATTCTGTGCTGCCT |
| | | GGCGAGCGGCTCGTGCGAAACGGTGTCTCTTGGAGCCACAGC |
| | | TCCATGCTGCCTCTGGGAAGCTCTCTGCCTGATGAGCTGCTGT |
| | | TCGCCGACGATTCTAGCGAGGGATCCGAGGTGCTGATGTGAtta |
| | | attaactgtgccttctagttgccagccatctgttgtttgcccctcccccgtgccttccttgaccctggaaggt |
| | | gccactcccactgtcctttcctaataaaatgaggaaattgcatcgcattgtctgagtaggtgtcattctattc |
| | | tggggggtggggtggggcaggacagcaagggggaggattgggaagacaatagcaggcatgctgg |
| | | ggaggcgcgccttctacgtagataagtagcatggcgggttaatcattaactacaaggaacccctagtga |
| | | tggagttggccactccctctctgcgcgctcgctcgctcactgaggccgggcgaccaaaggtcgcccg |
| | | acgcccgggctttgcccgggcggcctcagtgagcgagcgagcgcgcagGTTTAAACATC |
| | | CCAATGGCGCGCCGAGCTTGGCTCGAGCATGGTCATAGCTGTT |
| | | TCCTGTGTGAAATTGTTATCCGCTCACAATTCCACACAACATA |
| | | CGAGCCGGAAGCATAAAGTGTAAAGCCTGGGGTGCCTAATGA |
| | | GTGAGCTAACTCACATTAATTGCGTTGCGCTCACTGCCCGCTT |
| | | TCCAGTCGGGAAACCTGTCGTGCCAGCTGCATTAATGAATCGG |
| | | CCAACGCGCGGGGAGAGGCGGTTTGCGTATTGGGCGCTGTTC |
| | | CGCTTCCTCGCTCACTGACTCGCTGCGCTCGGTCGTTCGGCTG |
| | | CGGCGAGCGGTATCAGCTCACTCAAAGGCGGTAATACGGTTA |
| | | TCCACAGAATCAGGGGATAACGCAGGAAAGAACATGTGAGCA |
| | | AAAGGCCAGCAAAAGGCCAGGAACCGTAAAAAGGCCGCGTT |
| | | GCTGGCGTTTTTCCATAGGCTCCGCCCCCCTGACGAGCATCAC |
| | | AAAAATCGACGCTCAAGTCAGAGGTGGCGAAACCCGACAGGA |
| | | CTATAAAGATACCAGGCGTTTCCCCCTGGAAGCTCCCTCGTGC |
| | | GCTCTCCTGTTCCGACCCTGCCGCTTACCGGATACCTGTCCGC |
| | | CTTTCTCCCTTCGGGAAGCGTGGCGCTTTCTCATAGCTCACGC |
| | | TGTAGGTATCTCAGTTCGGTGTAGGTCGTTCGCTCCAAGCTGG |
| | | GCTGTGTGCACGAACCCCCCGTTCAGCCCGACCGCTGCGCCTT |
| | | ATCCGGTAACTATCGTCTTGAGTCCAACCCGGTAAGACACGAC |
| | | TTATCGCCACTGGCAGCAGCCACTGGTAACAGGATTAGCAGA |
| | | GCGAGGTATGTAGGCGGTGCTACAGAGTTCTTGAAGTGGTGG |
| | | CCTAACTACGGCTACACTAGAAGAACAGTATTTGGTATCTGCG |
| | | CTCTGCTGAAGCCAGTTACCTTCGGAAAAAGAGTTGGTAGCTC |
| | | TTGATCCGGCAAACAAACCACCGCTGGTAGCGGTGGTTTTTTT |
| | | GTTTGCAAGCAGCAGATTACGCGCAGAAAAAAAGGATCTCAA |
| | | GAAGATCCTTTGATCTTTTCTACGGGGTCTGACGCTCAGTGGA |
| | | ACGAAAACTCACGTTAAGGGATTTTGGTCATGAGATTATCAA |
| | | AAAGGATCTTCACCTAGATCCTTTTAAATTAAAAATGAAGTTT |
| | | TAAATCAATCTAAAGTATATATGAGTAAACTTGGTCTGACAGT |
| | | TAGAAAAACTCATCGAGCATCAAATGAAACTGCAATTTATTC |
| | | ATATCAGGATTATCAATACCATATTTTTGAAAAAGCCGTTTCT |
| | | GTAATGAAGGAGAAAACTCACCGAGGCAGTTCCATAGGATGG |
| | | CAAGATCCTGGTATCGGTCTGCGATTCCGACTCGTCCAACATC |
| | | AATACAACCTATTAATTTCCCCTCGTCAAAAATAAGGTTATCA |
| | | AGTGAGAAATCACCATGAGTGACGACTGAATCCGGTGAGAAT |
| | | GGCAAAAGTTTATGCATTTCTTTCCAGACTTGTTCAACAGGCC |
| | | AGCCATTACGCTCGTCATCAAAATCACTCGCATCAACCAAACC |
| | | GTTATTCATTCGTGATTGCGCCTGAGCGAAACGAAATACGCGA |
| | | TCGCTGTTAAAAGGACAATTACAAACAGGAATCGAATGCAAC |
| | | CGGCGCAGGAACACTGCCAGCGCATCAACAATATTTTCACCT |
| | | GAATCAGGATATTCTTCTAATACCTGGAATGCTGTTTTCCCAG |
| | | GGATCGCAGTGGTGAGTAACCATGCATCATCAGGAGTACGGA |
| | | TAAAATGCTTGATGGTCGGAAGAGGCATAAATTCCGTCAGCC |
| | | AGTTTAGTCTGACCATCTCATCTGTAACATCATTGGCAACGCT |
| | | ACCTTTGCCATGTTTCAGAAACAACTCTGGCGCATCGGGCTTC |
| | | CCATACAATCGATAGATTGTCGCACCTGATTGCCCGACATTAT |
| | | CGCGAGCCCATTTATACCCATATAAATCAGCATCCATGTTGGA |
| | | ATTTAATCGCGGCCTAGAGCAAGACGTTTCCCGTTGAATATGG |
| | | CTCATACTCTTCCTTTTTCAATATTATTGAAGCATTTATCAGGG |
| | | TTATTGTCTCATGAGCGGATACATATTTGAATGTATTTAGAAA |
| | | AATAAACAAATAGGGGTTCCGCGCACATTTCCCCGAAAAGTG |
| | | CCACCTGACGTCTAAGAAACCATTATTATCATGACATTAACCT |
| | | ATAAAAATAGGCGTATCACGAGGCCCTTTTGTC |
| 8 | CBA- | TCGCGCGTTTCGGTGATGACGGTGAAAACCTCTGACACATGCA |
| | hMERTKwt | GCTCCCGGAGACTGTCACAGCTTGTCTGTAAGCGGATGCCGG |
| | | GAGCAGACAAGCCCGTCAGGGCGCGTCAGCGGGTGTTGGCGG |
| | | GTGTCGGGGCTGGCTTAACTATGCGGCATCAGAGCAGATTGT |
| | | ACTGAGAGTGCACCATATGCGGTGTGAAATACCGCACAGATG |
| | | CGTAAGGAGAAAATACCGCATCAGGCGCCATTCGCCATTCAG |
| | | GCTGCGCAACTGTTGGGAAGGGCGATCGGTGCGGGCCTCTTC |
| | | GCTATTACGCCAGCTGGCGAAAGGGGGATGTGCTGCAAGGCG |
| | | ATTAAGTTGGGTAACGCCAGGGTTTTCCCAGTCACGACGTTGT |
| | | AAAACGACGGCCAGTGAATTGACGCGTATTGGGATGTTTAAA |
| | | Cctgcgcgctcgctcgctcactgaggccgcccgggcaaagcccgggcgtcgggcgacctttggtcg |
| | | cccggcctcagtgagcgagcgagcgcgcagagagggagtggccaactccatcactaggggttcctt |
| | | gtagttaatgattaacccgccatgctacttatctacgtagcgcggccgcggacattgattattgactagtta |
| | | ttaatagtaatcaattacggggtcattagttcatagcccatatatggagttccgcgttacataacttacggta |
| | | aatggcccgcctggctgaccgcccaacgacccccgcccattgacgtcaataatgacgtatgttcccata |
| | | gtaacgccaatagggactttccattgacgtcaatgggtggagtatttacggtaaactgcccacttggcag |
| | | tacatcaagtgtatcatatgccaagtacgccccctattgacgtcaatgacggtaaatggcccgcctggca |
| | | ttatgcccagtacatgaccttatgggactttcctacttggcagtacatctacgtattagtcatcgctattacc |
| | | atggtcgaggtgagccccacgttctgcttcactctccccatctcccccccctccccacccccaattttgta |
| | | tttatttattttttaattattttgtgcagcgatgggggcggggggggggggggggcgcgcgccaggcgg |
| | | ggcggggcggggcgaggggcggggcggggcgaggcggagaggtgcggcggcagccaatcaga |
| | | gcggcgcgctccgaaagtttccttttatggcgaggcggcggcggcggcggcccTATAAAaagc |
| | | gaagcgcgcggcgggcgggagtcGctGcGttgcgatcgcaggtaagtttagtctttttgtcttttatttc |
| | | aggtcccggatccggtggtggtgcaaatcaaagaactgctcctcagtggatgttgcctttacttctaggc |
| | | ctgcaggccgccaccatggggccggccccgctgccgctgctgctgggcctcttcctccccgcgctct |
| | | ggcgtagagctatcactgaggcaagggaagaagccaagccttacccgctattcccgggaccttttcca |
| | | gggagcctgcaaactgaccacacaccgctgttatcccttcctcacgccagtgggtaccagcctgccttg |
| | | atgttttcaccaacccagcctggaagaccacatacaggaaacgtagccattccccaggtgacctctgtc |
| | | gaatcaaagcccctaccgcctcttgccttcaaacacacagttggacacataatactttctgaacataaag |
| | | gtgtcaaatttaattgctcaatcagtgtacctaatatataccaggacaccacaatttcttggtggaaagatg |
| | | ggaaggaattgcttggggcacatcatgcaattacacagttttatccagatgatgaagttacagcaataatc |
| | | gcttccttcagcataaccagtgtgcagcgttcagacaatgggtcgtatatctgtaagatgaaaataaaca |
| | | atgaagagatcgtgtctgatcccatctacatcgaagtacaaggacttcctcactttactaagcagcctgag |
| | | agcatgaatgtcaccagaaacacagccttcaacctcacctgtcaggctgtgggcccgcctgagcccgt |
| | | caacattttctgggttcaaaacagtagccgtgttaacgaacagcctgaaaaatccccctccgtgctaact |
| | | gttccaggcctgacggagatggcggtcttcagttgtgaggcccacaatgacaaagggctgaccgtgtc |
| | | caagggagtgcagatcaacatcaaagcaattccctccccaccaactgaagtcagcatccgtaacagca |
| | | ctgcacacagcattctgatctcctgggttcctggttttgatggatactccccgttcaggaattgcagcattc |
| | | aggtcaaggaagctgatccgctgagtaatggctcagtcatgatttttaacacctctgccttaccacatctgt |
| | | accaaatcaagcagctgcaagccctggctaattacagcattggtgtttcctgcatgaatgaaataggctg |
| | | gtctgcagtgagcccttggattctagccagcacgactgaaggagccccatcagtagcacctttaaatgt |
| | | cactgtgtttctgaatgaatctagtgataatgtggacatcagatggatgaagcctccgactaagcagcag |
| | | gatggagaactggtgggctaccggatatcccacgtgtggcagagtgcagggatttccaaagagctctt |
| | | ggaggaagttggccagaatggcagccgagctcggatctctgttcaagtccacaatgctacgtgcacag |
| | | tgaggattgcagccgtcaccagagggggagttgggcccttcagtgatccagtgaaaatatttatccctg |
| | | cacacggttgggtagattatgccccctcttcaactccggcgcctggcaacgcagatcctgtgctcatcat |
| | | ctttggctgcttttgtggatttattttgattgggttgattttatacatctccttggccatcagaaaaagagtcc |
| | | aggagacaaagtttgggaatgcattcacagaggaggattctgaattagtggtgaattatatagcaaagaa |
| | | atccttctgtcggcgagccattgaacttaccttacatagcttgggagtcagtgaggaactacaaaataaa |
| | | ctagaagatgttgtgattgacaggaatcttctaattcttggaaaaattctgggtgaaggagagtttgggtct |
| | | gtaatggaaggaaatcttaagcaggaagatgggacctctctgaaagtggcagtgaagaccatgaagtt |
| | | ggacaactcttcacagcgggagatcgaggagtttctcagtgaggcagcgtgcatgaaagacttcagcc |
| | | acccaaatgtcattcgacttctaggtgtgtgtatagaaatgagctctcaaggcatcccaaagcccatggt |
| | | aattttacccttcatgaaatacggggacctgcatacttacttactttattcccgattggagacaggaccaaa |
| | | gcatattcctctgcagacactattgaagttcatggtggatattgccctgggaatggagtatctgagcaaca |
| | | ggaattttcttcatcgagatttagctgctcgaaactgcatgttgcgagatgacatgactgtctgtgttgcgg |
| | | acttcggcctctctaagaagatttacagtggcgattattaccgccaaggccgcattgctaagatgcctgtt |
| | | aaatggatcgccatagaaagtcttgcagaccgagtctacacaagtaaaagtgatgtgtgggcatttggc |
| | | gtgaccatgtgggaaatagctacgcggggaatgactccctatcctggggtccagaaccatgagatgtat |
| | | gactatcttctccatggccacaggttgaagcagcccgaagactgcctggatgaactgtatgaaataatgt |
| | | actcttgctggagaaccgatcccttagaccgccccaccttttcagtattgaggctgcagctagaaaaact |
| | | cttagaaagtttgcctgacgttcggaaccaagcagacgttatttacgtcaatacacagttgctggagagct |
| | | ctgagggcctggcccagggctccacccttgctccactggacttgaacatcgaccctgactctataattgc |
| | | ctcctgcactccccgcgctgccatcagtgtggtcacagcagaagttcatgacagcaaacctcatgaag |
| | | gacggtacatcctgaatgggggcagtgaggaatgggaagatctgacttctgccccctctgctgcagtc |
| | | acagctgaaaagaacagtgttttaccgggggagagacttgttaggaatggggtctcctggtcccattcg |
| | | agcatgctgcccttgggaagctcattgcccgatgaacttttgtttgctgacgactcctcagaaggctcag |
| | | aagtcctgatgtgattaattaactgtgccttctagttgccagccatctgttgtttgcccctcccccgtgcctt |
| | | ccttgaccctggaaggtgccactcccactgtcctttcctaataaaatgaggaaattgcatcgcattgtctga |
| | | gtaggtgtcattctattctggggggtggggtggggcaggacagcaagggggaggattgggaagaca |
| | | atagcaggcatgctggggaggcgcgccttctacgtagataagtagcatggcgggttaatcattaactac |
| | | aaggaacccctagtgatggagttggccactccctctctgcgcgctcgctcgctcactgaggccgggcg |
| | | accaaaggtcgcccgacgcccgggctttgcccgggcggcctcagtgagcgagcgagcgcgcagG |
| | | TTTAAACATCCCAATGGCGCGCCGAGCTTGGCTCGAGCATGGT |
| | | CATAGCTGTTTCCTGTGTGAAATTGTTATCCGCTCACAATTCC |
| | | ACACAACATACGAGCCGGAAGCATAAAGTGTAAAGCCTGGGG |
| | | TGCCTAATGAGTGAGCTAACTCACATTAATTGCGTTGCGCTCA |
| | | CTGCCCGCTTTCCAGTCGGGAAACCTGTCGTGCCAGCTGCATT |
| | | AATGAATCGGCCAACGCGCGGGGAGAGGCGGTTTGCGTATTG |
| | | GGCGCTGTTCCGCTTCCTCGCTCACTGACTCGCTGCGCTCGGT |
| | | CGTTCGGCTGCGGCGAGCGGTATCAGCTCACTCAAAGGCGGT |
| | | AATACGGTTATCCACAGAATCAGGGGATAACGCAGGAAAGAA |
| | | CATGTGAGCAAAAGGCCAGCAAAAGGCCAGGAACCGTAAAA |
| | | AGGCCGCGTTGCTGGCGTTTTTCCATAGGCTCCGCCCCCCTGA |
| | | CGAGCATCACAAAAATCGACGCTCAAGTCAGAGGTGGCGAAA |
| | | CCCGACAGGACTATAAAGATACCAGGCGTTTCCCCCTGGAAG |
| | | CTCCCTCGTGCGCTCTCCTGTTCCGACCCTGCCGCTTACCGGAT |
| | | ACCTGTCCGCCTTTCTCCCTTCGGGAAGCGTGGCGCTTTCTCAT |
| | | AGCTCACGCTGTAGGTATCTCAGTTCGGTGTAGGTCGTTCGCT |
| | | CCAAGCTGGGCTGTGTGCACGAACCCCCCGTTCAGCCCGACC |
| | | GCTGCGCCTTATCCGGTAACTATCGTCTTGAGTCCAACCCGGT |
| | | AAGACACGACTTATCGCCACTGGCAGCAGCCACTGGTAACAG |
| | | GATTAGCAGAGCGAGGTATGTAGGCGGTGCTACAGAGTTCTT |
| | | GAAGTGGTGGCCTAACTACGGCTACACTAGAAGAACAGTATT |
| | | TGGTATCTGCGCTCTGCTGAAGCCAGTTACCTTCGGAAAAAGA |
| | | GTTGGTAGCTCTTGATCCGGCAAACAAACCACCGCTGGTAGC |
| | | GGTGGTTTTTTTGTTTGCAAGCAGCAGATTACGCGCAGAAAAA |
| | | AAGGATCTCAAGAAGATCCTTTGATCTTTTCTACGGGGTCTGA |
| | | CGCTCAGTGGAACGAAAACTCACGTTAAGGGATTTTGGTCAT |
| | | GAGATTATCAAAAAGGATCTTCACCTAGATCCTTTTAAATTAA |
| | | AAATGAAGTTTTAAATCAATCTAAAGTATATATGAGTAAACTT |
| | | GGTCTGACAGTTAGAAAAACTCATCGAGCATCAAATGAAACT |
| | | GCAATTTATTCATATCAGGATTATCAATACCATATTTTTGAAA |
| | | AAGCCGTTTCTGTAATGAAGGAGAAAACTCACCGAGGCAGTT |
| | | CCATAGGATGGCAAGATCCTGGTATCGGTCTGCGATTCCGACT |
| | | CGTCCAACATCAATACAACCTATTAATTTCCCCTCGTCAAAAA |
| | | TAAGGTTATCAAGTGAGAAATCACCATGAGTGACGACTGAAT |
| | | CCGGTGAGAATGGCAAAAGTTTATGCATTTCTTTCCAGACTTG |
| | | TTCAACAGGCCAGCCATTACGCTCGTCATCAAAATCACTCGCA |
| | | TCAACCAAACCGTTATTCATTCGTGATTGCGCCTGAGCGAAAC |
| | | GAAATACGCGATCGCTGTTAAAAGGACAATTACAAACAGGAA |
| | | TCGAATGCAACCGGCGCAGGAACACTGCCAGCGCATCAACAA |
| | | TATTTTCACCTGAATCAGGATATTCTTCTAATACCTGGAATGC |
| | | TGTTTTCCCAGGGATCGCAGTGGTGAGTAACCATGCATCATCA |
| | | GGAGTACGGATAAAATGCTTGATGGTCGGAAGAGGCATAAAT |
| | | TCCGTCAGCCAGTTTAGTCTGACCATCTCATCTGTAACATCAT |
| | | TGGCAACGCTACCTTTGCCATGTTTCAGAAACAACTCTGGCGC |
| | | ATCGGGCTTCCCATACAATCGATAGATTGTCGCACCTGATTGC |
| | | CCGACATTATCGCGAGCCCATTTATACCCATATAAATCAGCAT |
| | | CCATGTTGGAATTTAATCGCGGCCTAGAGCAAGACGTTTCCCG |
| | | TTGAATATGGCTCATACTCTTCCTTTTTCAATATTATTGAAGCA |
| | | TTTATCAGGGTTATTGTCTCATGAGCGGATACATATTTGAATG |
| | | TATTTAGAAAAATAAACAAATAGGGGTTCCGCGCACATTTCCC |
| | | CGAAAAGTGCCACCTGACGTCTAAGAAACCATTATTATCATG |
| | | ACATTAACCTATAAAAATAGGCGTATCACGAGGCCCTTTTGTC |
| 9 | VMD2- | TCGCGCGTTTCGGTGATGACGGTGAAAACCTCTGACACATGCA |
| | hMERTK- | GCTCCCGGAGACTGTCACAGCTTGTCTGTAAGCGGATGCCGG |
| | codop | GAGCAGACAAGCCCGTCAGGGCGCGTCAGCGGGTGTTGGCGG |
| | | GTGTCGGGGCTGGCTTAACTATGCGGCATCAGAGCAGATTGT |
| | | ACTGAGAGTGCACCATATGCGGTGTGAAATACCGCACAGATG |
| | | CGTAAGGAGAAAATACCGCATCAGGCGCCATTCGCCATTCAG |
| | | GCTGCGCAACTGTTGGGAAGGGCGATCGGTGCGGGCCTCTTC |
| | | GCTATTACGCCAGCTGGCGAAAGGGGGATGTGCTGCAAGGCG |
| | | ATTAAGTTGGGTAACGCCAGGGTTTTCCCAGTCACGACGTTGT |
| | | AAAACGACGGCCAGTGAATTGACGCGTATTGGGATGTTTAAA |
| | | Cctgcgcgctcgctcgctcactgaggccgcccgggcaaagcccgggcgtcgggcgacctttggtcg |
| | | cccggcctcagtgagcgagcgagcgcgcagagagggagtggccaactccatcactaggggttcctt |
| | | gtagttaatgattaacccgccatgctacttatctacgtagcgcggccgccgtcagcatatgcagaattctg |
| | | tcattttactagggtgatgaaattcccaagcaacaccatccttttcagataagggcactgaggctgagag |
| | | aggagctgaaacctacccggggtcaccacacacaggtggcaaggctgggaccagaaaccaggact |
| | | gttgactgcagcccggtattcattctttccatagcccacagggctgtcaaagaccccagggcctagtca |
| | | gaggctcctccttcctggagagttcctggcacagaagttgaagctcagcacagccccctaacccccaa |
| | | ctctctctgcaaggcctcaggggtcagaacactggtggagcagatcctttagcctctggattttagggcc |
| | | atggtagagggggtgttgccctaaattccagccctggtctcagcccaacaccctccaagaagaaattag |
| | | aggggccatggccaggctgtgctagccgttgcttctgagcagattacaagaagggactaagacaagg |
| | | actcctttgtggaggtcctggcttagggagtcaagtgacggcggctcagcactcacgtgggcagtgcc |
| | | agcctctaagagtgggcaggggcactggccacagagtcccagggagtcccaccagcctagtcgcca |
| | | gaccgcgatcgcaggtaagtttagtctttttgtcttttatttcaggtcccggatccggtggtggtgcaaatc |
| | | aaagaactgctcctcagtggatgttgcctttacttctaggcctgcaggccgccaccATGGGACC |
| | | TGCTCCTCTGCCTCTGCTGCTGGGACTGTTTCTGCCTGCTCTTT |
| | | GGCGGAGAGCCATCACCGAGGCCAGAGAGGAAGCCAAGCCTT |
| | | ATCCTCTGTTCCCCGGACCTTTTCCAGGCAGCCTGCAGACCGA |
| | | TCACACCCCTTTGCTGTCTCTGCCTCACGCCTCTGGCTATCAGC |
| | | CCGCTCTGATGTTCAGCCCCACACAGCCAGGCAGACCTCACAC |
| | | AGGCAATGTGGCCATTCCTCAAGTGACCAGCGTGGAAAGCAA |
| | | GCCCTTGCCTCCTCTGGCCTTCAAGCACACAGTGGGCCACATC |
| | | ATCCTGAGCGAGCACAAGGGCGTGAAGTTCAACTGCAGCATC |
| | | AGCGTGCCCAACATCTACCAGGACACCACCATCAGCTGGTGG |
| | | AAGGACGGCAAAGAACTGCTGGGAGCCCACCACGCCATCACA |
| | | CAGTTCTACCCCGACGATGAAGTGACCGCCATCATTGCCAGCT |
| | | TCAGCATCACCAGCGTGCAGAGAAGCGACAACGGCAGCTACA |
| | | TCTGCAAGATGAAGATCAACAACGAGGAAATCGTCAGCGACC |
| | | CCATCTACATCGAGGTGCAGGGCCTGCCTCACTTCACCAAGCA |
| | | GCCCGAGAGCATGAACGTGACCAGAAACACCGCCTTCAACCT |
| | | GACCTGTCAGGCCGTGGGACCTCCTGAGCCTGTGAACATCTTC |
| | | TGGGTGCAGAACAGCTCCAGAGTGAACGAGCAGCCTGAGAAG |
| | | TCCCCTAGCGTGCTGACAGTGCCTGGACTGACAGAGATGGCC |
| | | GTGTTTTCTTGCGAGGCCCACAACGACAAGGGCCTGACCGTGT |
| | | CTAAGGGCGTGCAGATCAATATCAAGGCCATTCCATCTCCACC |
| | | TACCGAGGTGTCCATCCGGAATAGCACAGCCCACTCCATCCTG |
| | | ATCTCTTGGGTGCCCGGCTTCGATGGCTACAGCCCCTTCAGAA |
| | | ACTGCTCCATCCAAGTGAAAGAGGCCGATCCTCTGAGCAACG |
| | | GCTCCGTGATGATCTTCAACACAAGCGCCCTGCCACACCTGTA |
| | | CCAGATCAAACAGCTGCAGGCTCTGGCCAACTACTCCATCGG |
| | | CGTGTCCTGCATGAACGAGATCGGCTGGAGTGCCGTGTCTCCT |
| | | TGGATCCTGGCCAGCACAACTGAAGGCGCTCCATCTGTGGCCC |
| | | CTCTGAATGTGACCGTGTTCCTGAACGAGAGCAGCGACAATG |
| | | TGGACATCCGGTGGATGAAGCCACCTACAAAGCAGCAGGACG |
| | | GCGAACTCGTGGGCTACAGAATCTCTCACGTGTGGCAGTCTGC |
| | | CGGCATCTCCAAAGAACTCCTCGAAGAAGTGGGCCAGAACGG |
| | | CAGCAGAGCCAGGATCTCTGTGCAGGTCCACAACGCCACATG |
| | | CACAGTGCGGATTGCCGCTGTGACAAGAGGCGGAGTGGGCCC |
| | | TTTTAGCGACCCCGTGAAGATCTTTATCCCCGCTCACGGCTGG |
| | | GTCGACTACGCCCCATCTTCTACACCAGCTCCAGGCAACGCTG |
| | | ACCCCGTGCTGATCATCTTCGGCTGCTTTTGCGGCTTTATCCTG |
| | | ATCGGCCTGATCCTGTACATCAGCCTGGCCATCAGAAAGCGG |
| | | GTGCAAGAGACAAAGTTCGGCAACGCCTTCACCGAAGAGGAC |
| | | AGCGAGCTGGTGGTCAACTATATCGCCAAGAAGTCCTTCTGCA |
| | | GACGGGCCATCGAGCTGACACTGCACAGTCTGGGAGTGTCCG |
| | | AGGAACTGCAGAACAAGCTGGAAGATGTGGTCATCGACCGGA |
| | | ACCTGCTGATCCTGGGCAAGATTCTCGGCGAGGGCGAGTTTG |
| | | GCTCTGTGATGGAAGGCAACCTGAAGCAAGAGGACGGCACCT |
| | | CTCTGAAGGTGGCCGTGAAAACCATGAAGCTGGACAACAGCA |
| | | GCCAGCGCGAGATCGAAGAGTTTCTGTCTGAGGCCGCCTGTAT |
| | | GAAGGATTTCTCTCACCCCAACGTGATCCGGCTGCTGGGCGTG |
| | | TGTATCGAGATGTCTAGCCAGGGCATCCCCAAGCCTATGGTCA |
| | | TCCTGCCTTTCATGAAGTACGGCGATCTGCACACCTACCTGCT |
| | | GTACTCCAGACTGGAAACAGGCCCCAAGCACATCCCTCTGCA |
| | | GACCCTGCTGAAGTTCATGGTGGATATCGCCCTCGGCATGGAA |
| | | TACCTGAGCAACCGGAACTTCCTGCACCGCGATCTGGCCGCCA |
| | | GAAATTGCATGCTGAGGGACGACATGACCGTGTGCGTGGCCG |
| | | ATTTTGGCCTGAGCAAGAAGATCTACAGCGGCGACTACTACC |
| | | GGCAGGGCAGAATTGCCAAGATGCCCGTGAAGTGGATCGCCA |
| | | TCGAGAGCCTGGCCGACAGAGTGTACACCAGCAAGTCTGACG |
| | | TGTGGGCCTTCGGCGTGACCATGTGGGAGATTGCCACCAGAG |
| | | GCATGACCCCTTATCCTGGCGTCCAGAACCACGAGATGTACG |
| | | ATTACCTGCTGCACGGCCACAGACTGAAGCAGCCAGAGGATT |
| | | GCCTGGACGAGCTGTACGAGATCATGTACTCTTGCTGGCGGAC |
| | | CGATCCACTGGACAGACCTACATTCTCCGTGCTGCGGCTGCAG |
| | | CTGGAAAAACTGCTGGAAAGCCTGCCTGACGTGCGGAACCAG |
| | | GCCGATGTGATCTACGTGAACACCCAGCTGCTGGAATCCAGC |
| | | GAAGGACTGGCCCAGGGATCTACACTGGCTCCTCTGGACCTG |
| | | AACATCGACCCCGACAGCATTATCGCCAGCTGCACACCAAGA |
| | | GCCGCCATCAGCGTTGTGACAGCCGAGGTGCACGATAGCAAG |
| | | CCTCACGAAGGCCGGTACATCCTGAATGGCGGAAGCGAGGAA |
| | | TGGGAAGATCTGACCAGCGCTCCTTCTGCCGCCGTGACCGCCG |
| | | AGAAAAATTCTGTGCTGCCTGGCGAGCGGCTCGTGCGAAACG |
| | | GTGTCTCTTGGAGCCACAGCTCCATGCTGCCTCTGGGAAGCTC |
| | | TCTGCCTGATGAGCTGCTGTTCGCCGACGATTCTAGCGAGGGA |
| | | TCCGAGGTGCTGATGTGAttaattaactgtgccttctagttgccagccatctgttgtttgcc |
| | | cctcccccgtgccttccttgaccctggaaggtgccactcccactgtcctttcctaataaaatgaggaaatt |
| | | gcatcgcattgtctgagtaggtgtcattctattctggggggtggggtggggcaggacagcaaggggga |
| | | ggattgggaagacaatagcaggcatgctggggaggcgcgccttctacgtagataagtagcatggcgg |
| | | gttaatcattaactacaaggaacccctagtgatggagttggccactccctctctgcgcgctcgctcgctc |
| | | actgaggccgggcgaccaaaggtcgcccgacgcccgggctttgcccgggggcctcagtgagcga |
| | | gcgagcgcgcagGTTTAAACATCCCAATGGCGCGCCGAGCTTGGCT |
| | | CGAGCATGGTCATAGCTGTTTCCTGTGTGAAATTGTTATCCGC |
| | | TCACAATTCCACACAACATACGAGCCGGAAGCATAAAGTGTA |
| | | AAGCCTGGGGTGCCTAATGAGTGAGCTAACTCACATTAATTGC |
| | | GTTGCGCTCACTGCCCGCTTTCCAGTCGGGAAACCTGTCGTGC |
| | | CAGCTGCATTAATGAATCGGCCAACGCGCGGGGAGAGGCGGT |
| | | TTGCGTATTGGGCGCTGTTCCGCTTCCTCGCTCACTGACTCGCT |
| | | GCGCTCGGTCGTTCGGCTGCGGCGAGCGGTATCAGCTCACTCA |
| | | AAGGCGGTAATACGGTTATCCACAGAATCAGGGGATAACGCA |
| | | GGAAAGAACATGTGAGCAAAAGGCCAGCAAAAGGCCAGGAA |
| | | CCGTAAAAAGGCCGCGTTGCTGGCGTTTTTCCATAGGCTCCGC |
| | | CCCCCTGACGAGCATCACAAAAATCGACGCTCAAGTCAGAGG |
| | | TGGCGAAACCCGACAGGACTATAAAGATACCAGGCGTTTCCC |
| | | CCTGGAAGCTCCCTCGTGCGCTCTCCTGTTCCGACCCTGCCGC |
| | | TTACCGGATACCTGTCCGCCTTTCTCCCTTCGGGAAGCGTGGC |
| | | GCTTTCTCATAGCTCACGCTGTAGGTATCTCAGTTCGGTGTAG |
| | | GTCGTTCGCTCCAAGCTGGGCTGTGTGCACGAACCCCCCGTTC |
| | | AGCCCGACCGCTGCGCCTTATCCGGTAACTATCGTCTTGAGTC |
| | | CAACCCGGTAAGACACGACTTATCGCCACTGGCAGCAGCCAC |
| | | TGGTAACAGGATTAGCAGAGCGAGGTATGTAGGCGGTGCTAC |
| | | AGAGTTCTTGAAGTGGTGGCCTAACTACGGCTACACTAGAAG |
| | | AACAGTATTTGGTATCTGCGCTCTGCTGAAGCCAGTTACCTTC |
| | | GGAAAAAGAGTTGGTAGCTCTTGATCCGGCAAACAAACCACC |
| | | GCTGGTAGCGGTGGTTTTTTTGTTTGCAAGCAGCAGATTACGC |
| | | GCAGAAAAAAAGGATCTCAAGAAGATCCTTTGATCTTTTCTAC |
| | | GGGGTCTGACGCTCAGTGGAACGAAAACTCACGTTAAGGGAT |
| | | TTTGGTCATGAGATTATCAAAAAGGATCTTCACCTAGATCCTT |
| | | TTAAATTAAAAATGAAGTTTTAAATCAATCTAAAGTATATATG |
| | | AGTAAACTTGGTCTGACAGTTAGAAAAACTCATCGAGCATCA |
| | | AATGAAACTGCAATTTATTCATATCAGGATTATCAATACCATA |
| | | TTTTTGAAAAAGCCGTTTCTGTAATGAAGGAGAAAACTCACCG |
| | | AGGCAGTTCCATAGGATGGCAAGATCCTGGTATCGGTCTGCG |
| | | ATTCCGACTCGTCCAACATCAATACAACCTATTAATTTCCCCT |
| | | CGTCAAAAATAAGGTTATCAAGTGAGAAATCACCATGAGTGA |
| | | CGACTGAATCCGGTGAGAATGGCAAAAGTTTATGCATTTCTTT |
| | | CCAGACTTGTTCAACAGGCCAGCCATTACGCTCGTCATCAAAA |
| | | TCACTCGCATCAACCAAACCGTTATTCATTCGTGATTGCGCCT |
| | | GAGCGAAACGAAATACGCGATCGCTGTTAAAAGGACAATTAC |
| | | AAACAGGAATCGAATGCAACCGGCGCAGGAACACTGCCAGCG |
| | | CATCAACAATATTTTCACCTGAATCAGGATATTCTTCTAATAC |
| | | CTGGAATGCTGTTTTCCCAGGGATCGCAGTGGTGAGTAACCAT |
| | | GCATCATCAGGAGTACGGATAAAATGCTTGATGGTCGGAAGA |
| | | GGCATAAATTCCGTCAGCCAGTTTAGTCTGACCATCTCATCTG |
| | | TAACATCATTGGCAACGCTACCTTTGCCATGTTTCAGAAACAA |
| | | CTCTGGCGCATCGGGCTTCCCATACAATCGATAGATTGTCGCA |
| | | CCTGATTGCCCGACATTATCGCGAGCCCATTTATACCCATATA |
| | | AATCAGCATCCATGTTGGAATTTAATCGCGGCCTAGAGCAAG |
| | | ACGTTTCCCGTTGAATATGGCTCATACTCTTCCTTTTTCAATAT |
| | | TATTGAAGCATTTATCAGGGTTATTGTCTCATGAGCGGATACA |
| | | TATTTGAATGTATTTAGAAAAATAAACAAATAGGGGTTCCGC |
| | | GCACATTTCCCCGAAAAGTGCCACCTGACGTCTAAGAAACCA |
| | | TTATTATCATGACATTAACCTATAAAAATAGGCGTATCACGAG |
| | | GCCCTTTTGTC |
| 10 | VMD2- | TCGCGCGTTTCGGTGATGACGGTGAAAACCTCTGACACATGCA |
| | hMERTKwt | GCTCCCGGAGACTGTCACAGCTTGTCTGTAAGCGGATGCCGG |
| | | GAGCAGACAAGCCCGTCAGGGCGCGTCAGCGGGTGTTGGCGG |
| | | GTGTCGGGGCTGGCTTAACTATGCGGCATCAGAGCAGATTGT |
| | | ACTGAGAGTGCACCATATGCGGTGTGAAATACCGCACAGATG |
| | | CGTAAGGAGAAAATACCGCATCAGGCGCCATTCGCCATTCAG |
| | | GCTGCGCAACTGTTGGGAAGGGCGATCGGTGCGGGCCTCTTC |
| | | GCTATTACGCCAGCTGGCGAAAGGGGGATGTGCTGCAAGGCG |
| | | ATTAAGTTGGGTAACGCCAGGGTTTTCCCAGTCACGACGTTGT |
| | | AAAACGACGGCCAGTGAATTGACGCGTATTGGGATGTTTAAA |
| | | Cctgcgcgctcgctcgctcactgaggccgcccgggcaaagcccgggcgtcgggcgacctttggtcg |
| | | cccggcctcagtgagcgagcgagcgcgcagagagggagtggccaactccatcactaggggttcctt |
| | | gtagttaatgattaacccgccatgctacttatctacgtagcgcggccgccgtcagcatatgcagaattctg |
| | | tcattttactagggtgatgaaattcccaagcaacaccatccttttcagataagggcactgaggctgagag |
| | | aggagctgaaacctacccggggtcaccacacacaggtggcaaggctgggaccagaaaccaggact |
| | | gttgactgcagcccggtattcattctttccatagcccacagggctgtcaaagaccccagggcctagtca |
| | | gaggctcctccttcctggagagttcctggcacagaagttgaagctcagcacagccccctaacccccaa |
| | | ctctctctgcaaggcctcaggggtcagaacactggtggagcagatcctttagcctctggattttagggcc |
| | | atggtagagggggtgttgccctaaattccagccctggtctcagcccaacaccctccaagaagaaattag |
| | | aggggccatggccaggctgtgctagccgttgcttctgagcagattacaagaagggactaagacaagg |
| | | actcctttgtggaggtcctggcttagggagtcaagtgacggcggctcagcactcacgtgggcagtgcc |
| | | agcctctaagagtgggcaggggcactggccacagagtcccagggagtcccaccagcctagtcgcca |
| | | gaccgcgatcgcaggtaagtttagtctttttgtcttttatttcaggtcccggatccggtggtggtgcaaatc |
| | | aaagaactgctcctcagtggatgttgcctttacttctaggcctgcaggccgccaccatggggccggccc |
| | | cgctgccgctgctgctgggcctcttcctccccgcgctctggcgtagagctatcactgaggcaagggaa |
| | | gaagccaagccttacccgctattcccgggaccttttccagggagcctgcaaactgaccacacaccgct |
| | | gttatcccttcctcacgccagtgggtaccagcctgccttgatgttttcaccaacccagcctggaagacca |
| | | catacaggaaacgtagccattccccaggtgacctctgtcgaatcaaagcccctaccgcctcttgccttca |
| | | aacacacagttggacacataatactttctgaacataaaggtgtcaaatttaattgctcaatcagtgtaccta |
| | | atatataccaggacaccacaatttcttggtggaaagatgggaaggaattgcttggggcacatcatgcaat |
| | | tacacagttttatccagatgatgaagttacagcaataatcgcttccttcagcataaccagtgtgcagcgttc |
| | | agacaatgggtcgtatatctgtaagatgaaaataaacaatgaagagatcgtgtctgatcccatctacatc |
| | | gaagtacaaggacttcctcactttactaagcagcctgagagcatgaatgtcaccagaaacacagccttc |
| | | aacctcacctgtcaggctgtgggcccgcctgagcccgtcaacattttctgggttcaaaacagtagccgt |
| | | gttaacgaacagcctgaaaaatccccctccgtgctaactgttccaggcctgacggagatggcggtcttc |
| | | agttgtgaggcccacaatgacaaagggctgaccgtgtccaagggagtgcagatcaacatcaaagcaa |
| | | ttccctccccaccaactgaagtcagcatccgtaacagcactgcacacagcattctgatctcctgggttcct |
| | | ggttttgatggatactccccgttcaggaattgcagcattcaggtcaaggaagctgatccgctgagtaatg |
| | | gctcagtcatgatttttaacacctctgccttaccacatctgtaccaaatcaagcagctgcaagccctggct |
| | | aattacagcattggtgtttcctgcatgaatgaaataggctggtctgcagtgagcccttggattctagccag |
| | | cacgactgaaggagccccatcagtagcacctttaaatgtcactgtgtttctgaatgaatctagtgataatgt |
| | | ggacatcagatggatgaagcctccgactaagcagcaggatggagaactggtgggctaccggatatcc |
| | | cacgtgtggcagagtgcagggatttccaaagagctcttggaggaagttggccagaatggcagccgag |
| | | ctcggatctctgttcaagtccacaatgctacgtgcacagtgaggattgcagccgtcaccagaggggga |
| | | gttgggcccttcagtgatccagtgaaaatatttatccctgcacacggttgggtagattatgccccctcttca |
| | | actccggcgcctggcaacgcagatcctgtgctcatcatctttggctgcttttgtggatttattttgattgggt |
| | | tgattttatacatctccttggccatcagaaaaagagtccaggagacaaagtttgggaatgcattcacagag |
| | | gaggattctgaattagtggtgaattatatagcaaagaaatccttctgtcggcgagccattgaacttacctta |
| | | catagcttgggagtcagtgaggaactacaaaataaactagaagatgttgtgattgacaggaatcttctaat |
| | | tcttggaaaaattctgggtgaaggagagtttgggtctgtaatggaaggaaatcttaagcaggaagatgg |
| | | gacctctctgaaagtggcagtgaagaccatgaagttggacaactcttcacagcgggagatcgaggagt |
| | | ttctcagtgaggcagcgtgcatgaaagacttcagccacccaaatgtcattcgacttctaggtgtgtgtata |
| | | gaaatgagctctcaaggcatcccaaagcccatggtaattttacccttcatgaaatacggggacctgcata |
| | | cttacttactttattcccgattggagacaggaccaaagcatattcctctgcagacactattgaagttcatggt |
| | | ggatattgccctgggaatggagtatctgagcaacaggaattttcttcatcgagatttagctgctcgaaact |
| | | gcatgttgcgagatgacatgactgtctgtgttgcggacttcggcctctctaagaagatttacagtggcgat |
| | | tattaccgccaaggccgcattgctaagatgcctgttaaatggatcgccatagaaagtcttgcagaccga |
| | | gtctacacaagtaaaagtgatgtgtgggcatttggcgtgaccatgtgggaaatagctacgcggggaatg |
| | | actccctatcctggggtccagaaccatgagatgtatgactatcttctccatggccacaggttgaagcagc |
| | | ccgaagactgcctggatgaactgtatgaaataatgtactcttgctggagaaccgatcccttagaccgccc |
| | | caccttttcagtattgaggctgcagctagaaaaactcttagaaagtttgcctgacgttcggaaccaagca |
| | | gacgttatttacgtcaatacacagttgctggagagctctgagggcctggcccagggctccacccttgctc |
| | | cactggacttgaacatcgaccctgactctataattgcctcctgcactccccgcgctgccatcagtgtggtc |
| | | acagcagaagttcatgacagcaaacctcatgaaggacggtacatcctgaatgggggcagtgaggaat |
| | | gggaagatctgacttctgccccctctgctgcagtcacagctgaaaagaacagtgttttaccgggggaga |
| | | gacttgttaggaatggggtctcctggtcccattcgagcatgctgcccttgggaagctcattgcccgatga |
| | | acttttgtttgctgacgactcctcagaaggctcagaagtcctgatgtgattaattaactgtgccttctagttg |
| | | ccagccatctgttgtttgcccctcccccgtgccttccttgaccctggaaggtgccactcccactgtcctttc |
| | | ctaataaaatgaggaaattgcatcgcattgtctgagtaggtgtcattctattctggggggtggggtgggg |
| | | caggacagcaagggggaggattgggaagacaatagcaggcatgctggggaggcgcgccttctacgt |
| | | agataagtagcatggcgggttaatcattaactacaaggaacccctagtgatggagttggccactccctct |
| | | ctgcgcgctcgctcgctcactgaggccgggcgaccaaaggtcgcccgacgcccgggctttgcccgg |
| | | gcggcctcagtgagcgagcgagcgcgcagGTTTAAACATCCCAATGGCGCGC |
| | | CGAGCTTGGCTCGAGCATGGTCATAGCTGTTTCCTGTGTGAAA |
| | | TTGTTATCCGCTCACAATTCCACACAACATACGAGCCGGAAGC |
| | | ATAAAGTGTAAAGCCTGGGGTGCCTAATGAGTGAGCTAACTC |
| | | ACATTAATTGCGTTGCGCTCACTGCCCGCTTTCCAGTCGGGAA |
| | | ACCTGTCGTGCCAGCTGCATTAATGAATCGGCCAACGCGCGG |
| | | GGAGAGGCGGTTTGCGTATTGGGCGCTGTTCCGCTTCCTCGCT |
| | | CACTGACTCGCTGCGCTCGGTCGTTCGGCTGCGGCGAGCGGTA |
| | | TCAGCTCACTCAAAGGCGGTAATACGGTTATCCACAGAATCA |
| | | GGGGATAACGCAGGAAAGAACATGTGAGCAAAAGGCCAGCA |
| | | AAAGGCCAGGAACCGTAAAAAGGCCGCGTTGCTGGCGTTTTT |
| | | CCATAGGCTCCGCCCCCCTGACGAGCATCACAAAAATCGACG |
| | | CTCAAGTCAGAGGTGGCGAAACCCGACAGGACTATAAAGATA |
| | | CCAGGCGTTTCCCCCTGGAAGCTCCCTCGTGCGCTCTCCTGTT |
| | | CCGACCCTGCCGCTTACCGGATACCTGTCCGCCTTTCTCCCTTC |
| | | GGGAAGCGTGGCGCTTTCTCATAGCTCACGCTGTAGGTATCTC |
| | | AGTTCGGTGTAGGTCGTTCGCTCCAAGCTGGGCTGTGTGCACG |
| | | AACCCCCCGTTCAGCCCGACCGCTGCGCCTTATCCGGTAACTA |
| | | TCGTCTTGAGTCCAACCCGGTAAGACACGACTTATCGCCACTG |
| | | GCAGCAGCCACTGGTAACAGGATTAGCAGAGCGAGGTATGTA |
| | | GGCGGTGCTACAGAGTTCTTGAAGTGGTGGCCTAACTACGGCT |
| | | ACACTAGAAGAACAGTATTTGGTATCTGCGCTCTGCTGAAGCC |
| | | AGTTACCTTCGGAAAAAGAGTTGGTAGCTCTTGATCCGGCAA |
| | | ACAAACCACCGCTGGTAGCGGTGGTTTTTTTGTTTGCAAGCAG |
| | | CAGATTACGCGCAGAAAAAAAGGATCTCAAGAAGATCCTTTG |
| | | ATCTTTTCTACGGGGTCTGACGCTCAGTGGAACGAAAACTCAC |
| | | GTTAAGGGATTTTGGTCATGAGATTATCAAAAAGGATCTTCAC |
| | | CTAGATCCTTTTAAATTAAAAATGAAGTTTTAAATCAATCTAA |
| | | AGTATATATGAGTAAACTTGGTCTGACAGTTAGAAAAACTCAT |
| | | CGAGCATCAAATGAAACTGCAATTTATTCATATCAGGATTATC |
| | | AATACCATATTTTTGAAAAAGCCGTTTCTGTAATGAAGGAGAA |
| | | AACTCACCGAGGCAGTTCCATAGGATGGCAAGATCCTGGTAT |
| | | CGGTCTGCGATTCCGACTCGTCCAACATCAATACAACCTATTA |
| | | ATTTCCCCTCGTCAAAAATAAGGTTATCAAGTGAGAAATCACC |
| | | ATGAGTGACGACTGAATCCGGTGAGAATGGCAAAAGTTTATG |
| | | CATTTCTTTCCAGACTTGTTCAACAGGCCAGCCATTACGCTCG |
| | | TCATCAAAATCACTCGCATCAACCAAACCGTTATTCATTCGTG |
| | | ATTGCGCCTGAGCGAAACGAAATACGCGATCGCTGTTAAAAG |
| | | GACAATTACAAACAGGAATCGAATGCAACCGGCGCAGGAACA |
| | | CTGCCAGCGCATCAACAATATTTTCACCTGAATCAGGATATTC |
| | | TTCTAATACCTGGAATGCTGTTTTCCCAGGGATCGCAGTGGTG |
| | | AGTAACCATGCATCATCAGGAGTACGGATAAAATGCTTGATG |
| | | GTCGGAAGAGGCATAAATTCCGTCAGCCAGTTTAGTCTGACC |
| | | ATCTCATCTGTAACATCATTGGCAACGCTACCTTTGCCATGTTT |
| | | CAGAAACAACTCTGGCGCATCGGGCTTCCCATACAATCGATA |
| | | GATTGTCGCACCTGATTGCCCGACATTATCGCGAGCCCATTTA |
| | | TACCCATATAAATCAGCATCCATGTTGGAATTTAATCGCGGCC |
| | | TAGAGCAAGACGTTTCCCGTTGAATATGGCTCATACTCTTCCT |
| | | TTTTCAATATTATTGAAGCATTTATCAGGGTTATTGTCTCATGA |
| | | GCGGATACATATTTGAATGTATTTAGAAAAATAAACAAATAG |
| | | GGGTTCCGCGCACATTTCCCCGAAAAGTGCCACCTGACGTCTA |
| | | AGAAACCATTATTATCATGACATTAACCTATAAAAATAGGCGT |
| | | ATCACGAGGCCCTTTTGTC |

TABLE 4
MERTK Primer Sequences

| SEQ ID | | |
|---|---|---|
| NO | Sequence Name | Sequence |
| 11 | MERTKwt primer | 5′-/56-FAM/CCACTGGACTTGAACATCGACCCT/ |
| | 1 | 36-TAMSp/-3′ |
| 12 | MERTKwt primer | 5′-GGAGTGCAGGAGGCAATTAT-3′ |
| | 2 | |
| 13 | MERTKwt primer | 5′-TCAATACACAGTTGCTGGAGAG-3′ |
| | 3 | |
| 14 | MERTKcodop | 5′-/56-FAM/ATCGAGCTGACACTGCACAGTCTG/ |
| | primer 1 | 36-TAMSp/-3′ |
| 15 | MERTKcodop | 5′-GACCACATCTTCCAGCTTGT-3′ |
| | primer 2 | |
| 16 | MERTKcodop | 5′-CGAGCTGGTGGTCAACTATATC-3′ |
| | primer 3 | |

### Results

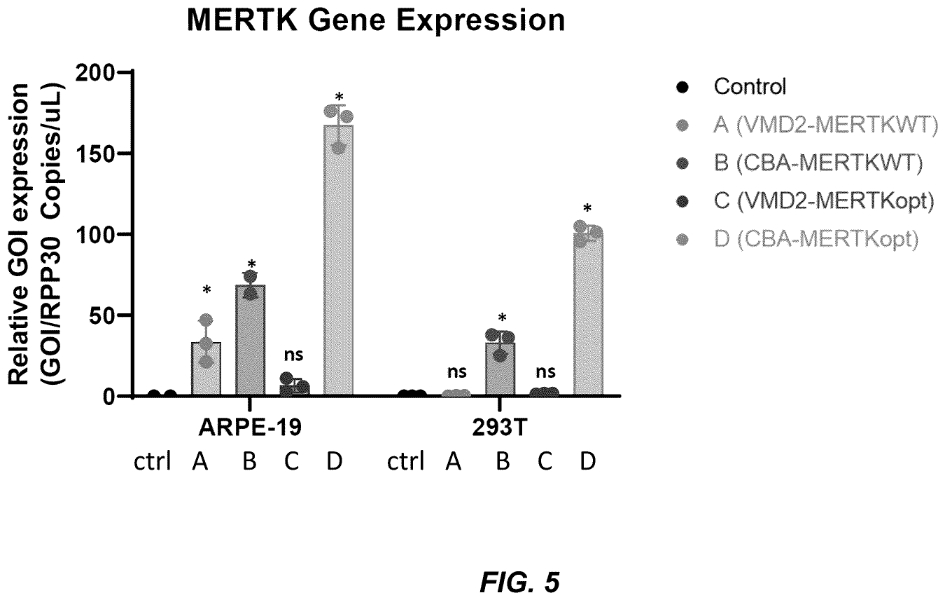

ARPE19 cells expressed extremely low levels of MERTK (˜21 copies/ng cDNA). Transfection with CBA-hMERTKwt, VMD2-hMERTKwt, and VMD2-hMERTKcodop led to significant upregulation of MERTK expression (700-fold, 250-fold, and 3-fold, respectively) as compared to control transfected cells. Consistent with the gene expression data, ARPE19 cells transfected with CBA-hMERTKwt, CBA-hMERTKcodop and VMD2-hMERTKcodop plasmids exhibited significant upregulation of MERTK protein expression. 293T cells exhibited 100-fold upregulation of MERTK expression as a result of transfection with CBA-hMERTKwt and expectedly, no significant induction with VMD2-hMERTKwt. See FIG. 5.

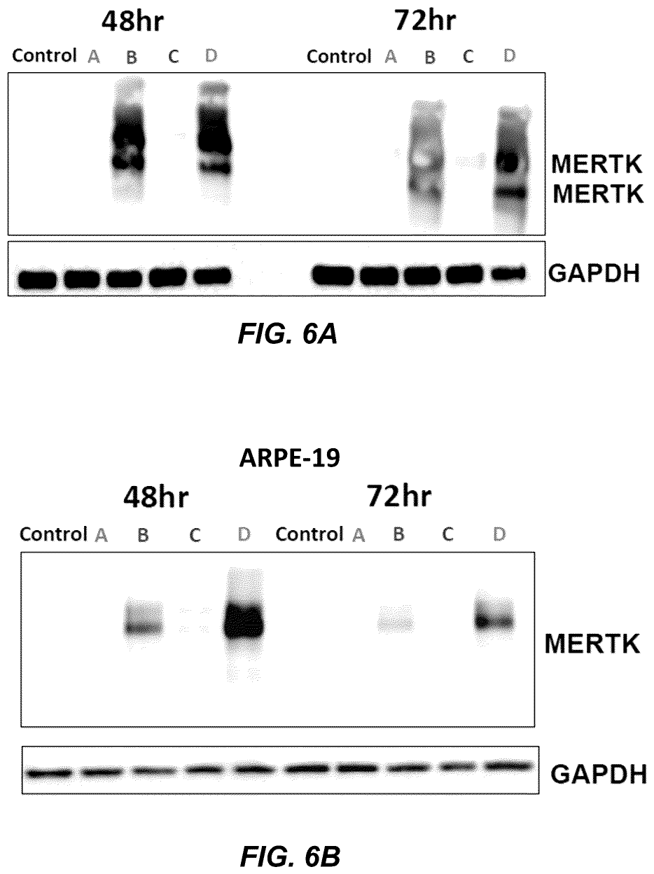

Gene expression results showed that CBA promoter-driven MERTK mRNA expression was relatively higher than VMD2 promoter-driven expression (FIG. 5 and Table 5). Western blot results showed that CBA promoter-driven MERTK protein expression was relatively higher than VMD2 promoter-driven expression (FIGS. 6A-6B).

TABLE 5
MERTK gene expression in response to transfection of MERTK constructs

| | | | VMD2- | CBA- | VMD2- | CBA- |
|---|---|---|---|---|---|---|
| | | Control | MERTKWT | MERTKWT | MERTKopt | MERTKopt |
| ARPE19 | Mean | 0.07 | 33.66 | 68.67 | 6.50 | 167.34 |
| cells | SEM | 0.02 | 7.44 | 4.42 | 2.48 | 7.13 |
| 293T | Mean | 0.03 | 0.32 | 33.08 | 1.54 | 100.64 |
| cells | SEM | 0.00 | 0.08 | 4.05 | 0.19 | 2.69 |

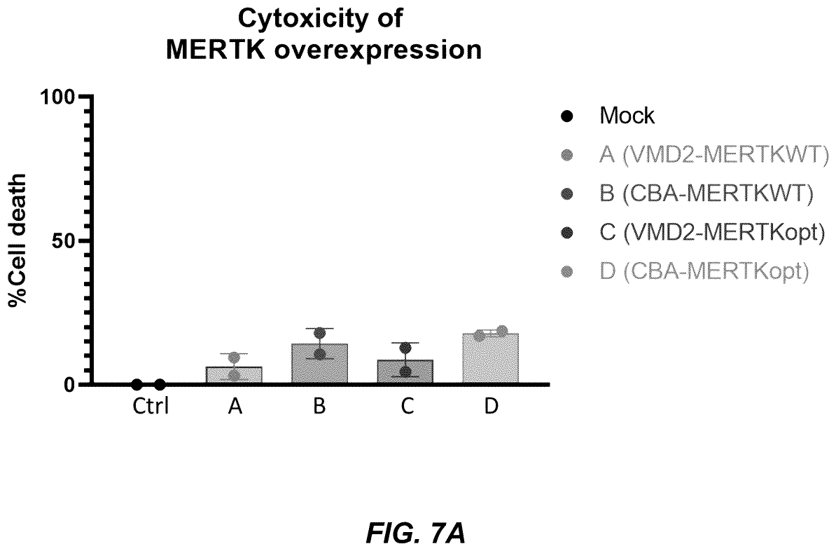

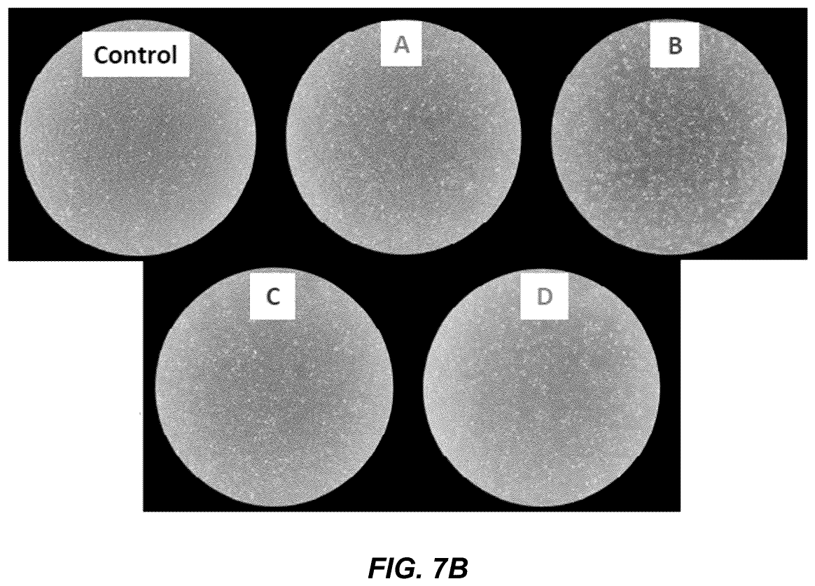

The impact of MERTK overexpression was assessed by measuring cell viability of ARPE19 cell post transfections. Plasmid transfection with MERTK-constructs results in modest cellular toxicity, which is not significantly different from control treated cells (FIGS. 7A-7B). ARPE-19 cells were reverse transfected with each plasmid in duplicate and then incubated for 48 hours. After treating the cells with CellTiter Glo 2.0, cell death was calculated by subtracting normalized RLU from 100, (100-[RLU/Control RLU]*100) (FIG. 7A). Cell density was visualized with phase-contrast microscopy (FIG. 7B).

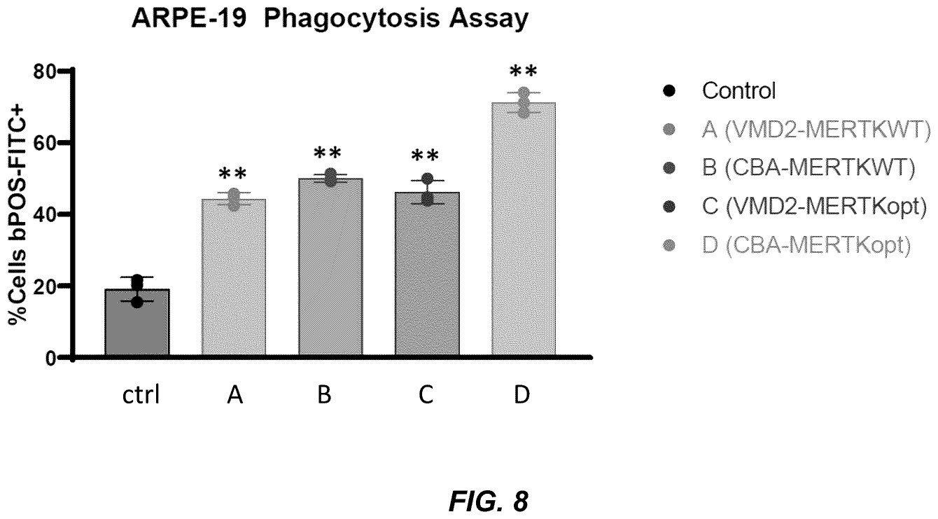

Since MERTK is critical regulator of POS phagocytosis by RPE cells, a flow cytometry-based assay was employed to measure the phagocytosis of FITC-labeled POS by ARPE19 cells. Transfected ARPE-19 cells, treated with bPOS-FITC were analyzed using Flow cytometry to determine the percentage of FITC+ cells, as a measure of MERTK-driven phagocytic activity. Treatment with all four constructs increases phagocytosis of POS over baseline control-transfected ARPE19 cells (FIG. 8). Transfection with all the tested plasmids led to a significant upregulation of POS internalization over the baseline. The CBA-MERTKopt plasmid showed the maximum upregulation of phagocytosis.

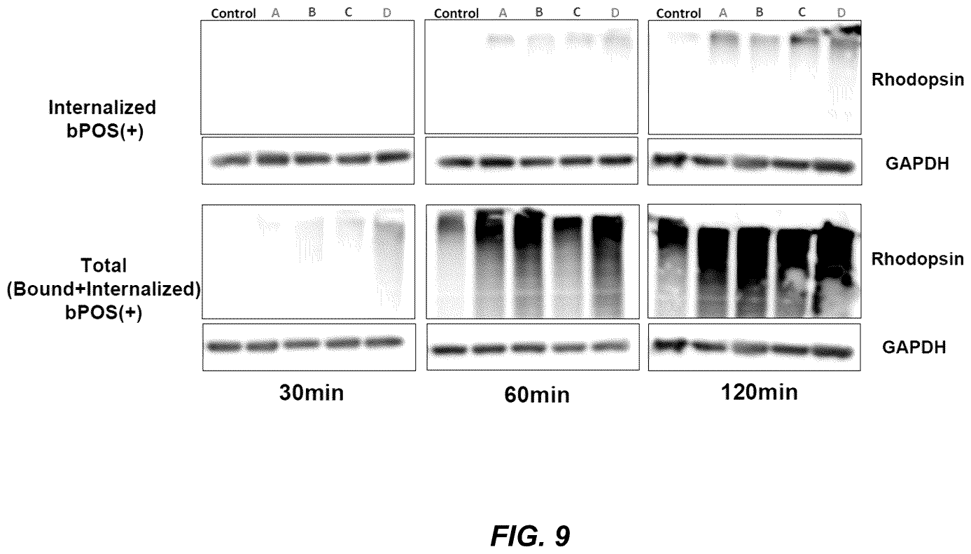

Additionally, a western blot-based assay measuring Rhodopsin internalization was used to assess internalization of POS by transfected ARPE19 cells. A time-dependent increase in Rhodopsin internalization was observed in transfected ARPE19 cells, at 60 mins and 120 mins after incubation with bovine POS (FIG. 9). All four constructs exhibited an upregulation of Rhodopsin internalization in ARPE19 cells suggesting a functional augmentation as result to MERTK protein expression. Treatment with all four constructs increases phagocytosis of POS by RPE cells over baseline control-transfected ARPE19 cells.

Rhodopsin internalization from cell lysates of ARPE-19 cells was assayed using cells washed twice, treated with trypsin and washed twice before being lysed. Transfected ARPE19 cells were incubated with bPOS (bPOS(+)) and control cells were not incubated with bPOS (bPOS (−)) 5 μg of protein was used for SDS-PAGE followed by immunoblotting with rhodopsin (˜36 kDA) and a loading control, GAPDH (˜36 kDa). Total rhodopsin from cell lysates of ARPE-19 cells was also assayed using cells washed twice then lysed directly without trypsinization). 5 μg of protein was used for SDS-PAGE followed by immunoblotting with rhodopsin (˜36 kDA) and a loading control, GAPDH (˜36 kDa). (FIG. 9).

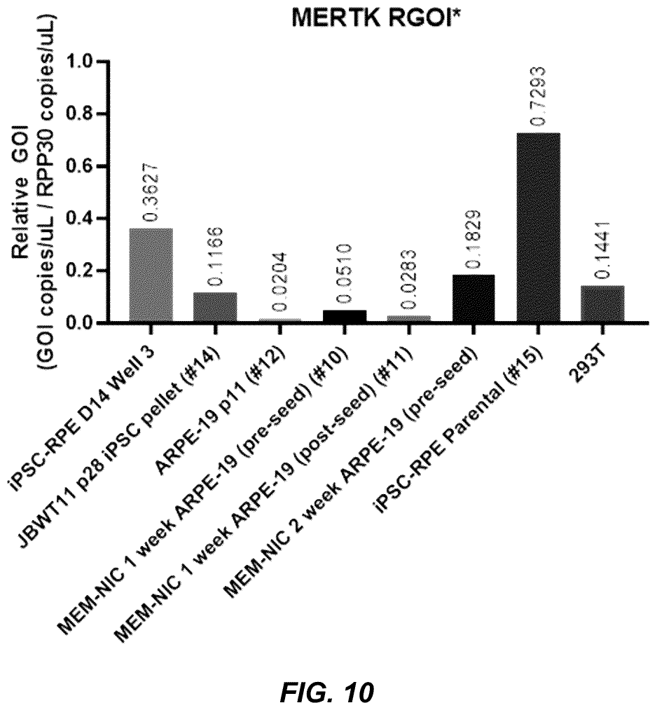

MERTK expression was quantified in the RPE-cell culture models, as depicted in FIG. 10. It was observed that ARPE19 cells had relatively lower baseline expression than iPSC-RPE cells.

The plasmid construct driving expression of the codon optimized MERTK sequence under the control of CBA promoter significantly increased MERTK mRNA and protein expression as compared to the constructs with the VMD2 promoter. ARPE19 cells exhibited a low baseline phagocytic activity which was significantly upregulated by all four constructs. Additionally, all four constructs showed an increase internalization of bPOS relative to control, as measured by rhodopsin immunoblotting.

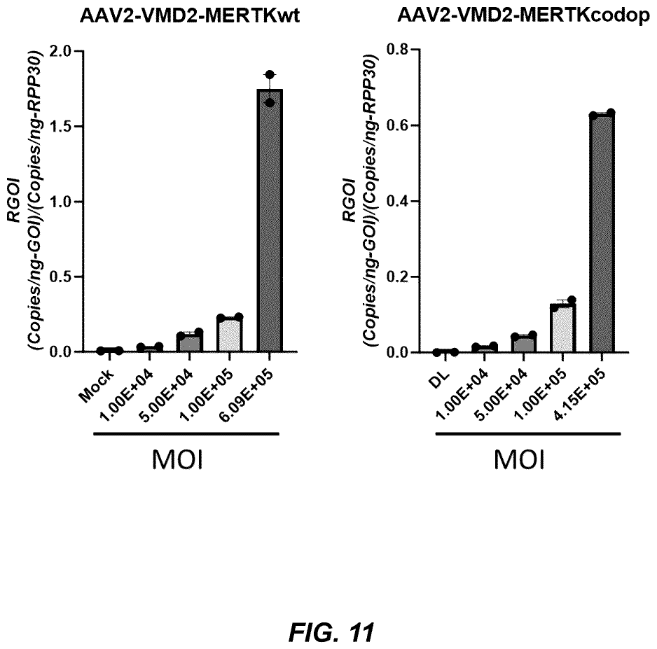

Induction of MERTK expression was assessed in ARPE19 cells 48 hours post-transduction with AAV2-VMD2-MERTKwt and AAV2-VMD2-MERTKcodop. As depicted in FIG. 11, transduction with both the AAVs induced the expression of their relative MERTK sequences (native and codop) in ARPE19 cells. In vitro proof-of-concept data supporting the efficacy of AAV vectors in inducing the expression of native and codop MERTK in ARPE19 cells is also provided.

The results showed that rAAV2-hMERTK constructs containing the CBA promoter and codon optimized sequence induced higher expression than the VMD2 promoter and wild-type sequence but exhibited comparable rescue of POS internalization in ARPE19 cells.

### Example 2: Administration of MERTK Gene Therapy in Mice

Plasmid constructs from Example 1 are placed into recombinant AAV vectors and tested in MERTK+ mice for their ability to induce RPE-specific transgene expression and rescue of retinal degradation.

Mice are bred and maintained in an animal care facility where they were fed 4% fat rodent diet and water ad libitum and housed in a 12-hour light/12-hour dark cycle. A tissue biopsy is prepared for polymerase chain reaction (PCR) using Allele-In-One Mouse Tail Direct Lysis Buffer, according to the manufacturer's instructions. PCR is performed out to amplify a region of MERTK. The 20 μL PCR reactions have final concentrations of 200 μmol/L for each primer, 200 nmol/L for each of the dNTPs (dATP, dGTP, dTTP, and dCTP), 2 mmol/L MgCl2, and I unit of Hot FirePol DNA polymerase. The thermocycling protocol is 95° C. for 14 minutes; 30 cycles of 95° C. for 45 seconds, 53° C. for 45 seconds, 72° C. for 30 seconds; 72° C. for 7 minutes. The amplified product is subjected to Sanger sequencing, and the electropherograms are analyzed at c.25 to identify each mouse as being wildtype, heterozygous, or homozygous for a MERTK mutation.

### DNA Construct and AAV Vector Preparation

Codon-optimized human MERTK cDNA is synthesized into a gBlock gene fragment and incorporated into constructs that are then packaged into recombinant AAV viral vectors.

Plasmids containing the full constructs are generated using standard endotoxin-free molecular cloning techniques and validated by sequencing MERTK and regions crossing ligation sites.

AAV is prepared, purified virus is collected in a final buffer containing 1×PBS, 35 mM NaCl, and 0.001% Pluronic F68 surfactant, and the purified virus is titered. The same buffer is used to further dilute the virus, if required, to achieve the target dose. To assist with the injection procedure, <0.25% of fluorescein (AK-Fluor, Akorn, Lake Forest, IL) is mixed into the working solution as a tracer.

For general anesthesia, a mixture of ketamine/xlyazine is delivered by intraperitoneal injection. Two-week-old mice receive a dose of 37.5 mg/kg ketamine and 3.8 mg/kg xylazine and adult mice receive a dose of 100 mg/kg ketamine and 20 mg/kg xylazine. To counteract the formation of permanent anesthesia-induced corneal opacities, a 2 mg/kg dose of Yohimbine HCL is administered by subcutaneous injection immediately following each recovery procedure in which ketamine/xylazine was used (i.e., AAV injection, in vivo imaging, ERG). For neonatal mice, general anesthesia by hypothermia was induced by indirect exposure to ice.

In two-week-old mice, a Micro4 microinjection pump with RPE kit is used to deliver the viral reagents into either the subretinal space or the vitreous chamber. Pupils are dilated using either Tropicamide (1%) or a half mixture of Tropicamide (0.25%), Phenyephrine hydrochloride (0.25%), and Cyclepentalate (1%). Mice are deeply anesthetized with ketamine/xylazine and local anesthesia is administered topically using Proparacaine hydrochloride (0.5%). Next, the eye is proposed and a 30 g syringe needle is used to puncture the superior-temporal sclera and retina immediately posterior to the episcleral vessels of limbus to make an entry route for a blunt-end 33 g cannula.

For subretinal injections, the traversal of the cannula through the vitreous chamber is visualized via a dissecting microscope through the dilated pupil and the cannula tip is positioned in the subretinal space of the posterior part of the inferior-nasal quadrant of the eye. Four successive 185.5 nL boluses of reagent (0.75 μL total) are injected, and the formation of a bleb is confirmed by visualization that is enhanced by the fluorescein tracer. The cannula is held in place for approximately three seconds following injection to avoid reflux of the reagent and then gently removed from the eye. Finally, the entry wound is treated by tamponade with a cotton swab. The eyes are then hydrated with artificial tears, and the mice recover from anesthesia on a heating pad.

The procedure for intravitreal injection of two-week-old mice is identical, except that the cannula tip is positioned in the center of the vitreous chamber during injection.

In neonates, subretinal injections are performed using the FemtoJet 4i microinjection system (Eppendorf, Hamburg, Germany). While the mice are anesthetized on ice, the tip of a 30 g hypodermic needle is used to separate the upper and lower eyelids. The eye is proptosed and a custom beveled glass needle (Cat #C060609, Origio, Trumbull, CT) is directly inserted through the sclera and positioned in the underlying subretinal space. A single bolus of 0.5 μL of reagent is administered at pressure of 330 hPa over 6 seconds, after which the needle is held in place for approximately three seconds to avoid reflux and then gently removed. The mice recover from anesthesia on a heating pad.

En face and cross-sectional images of the retina are acquired using fundus photography and spectral domain optical coherence tomography (OCT). Using in In VivoVue OCT software, four approximately equally spaced caliper measurements are made from the outer plexiform layer to the retinal pigment epithelium to measure photoreceptor layer thickness.

Full-field, flash electroretinograms are collected from the mice. Briefly, mice are dark adapted overnight, and rod and mixed rod/cone responses are generated using a 0.01 cd·s/m2 (scotopic) and 10 cd·s/m2 (scotopic) broadband light stimuli, respectively. Next, the mice are light adapted by exposure to a steady 30 cd/m² (photopic) broadband light for 10 minutes, and this light remains on in the background during the acquisition of cone-isolated responses to a 20 cd·s/m2 (photopic) broadband light stimulus.

Statistical analyses are completed in Prism. For OCT and electroretinogram (ERG) time courses, a two-way ANOVA using a mixed effects regression model is performed. When analyzing the effect of treatment, the inferior retina of the non-injected eye is used as the negative control to which the means all other measurements are compared. The Dunnett post hoc test is used to account for Type I error generated from multiple comparisons testing.

Additional data is collected from RPE-choroid flatmounts (showing tight junctions, morphology, and sub-retinal immune cells), retinal cross sections (showing MERTK/GFP expression), and tunneling electron microscopy (TEM) (showing RPE morphology, cellular vacuoles, outer-segment rosettes, and sub-RPE deposits).

### Example 3: Administration of MERTK Gene Therapy in Humans

In this example, codon-optimized MERTK sequences are injected into the eye to treat retinitis pigmentosa in human subjects.

Adeno-associated virus proviral plasmids are generated containing codon-optimized MERTK cDNA (e.g., SEQ ID NOs: 3-6). In these proviral plasmids, the hMERTK cDNA is driven by the VMD2 promoter. For example, a dose of the AAV2-VMD2-hMERTKcodop vector (e.g., SEQ ID NO: 9) can be administered to an eye of a human subject to treat retinitis pigmentosa.

The rAAV plasmids are administered by subretinal injection at 1010 viral particles in a suspension in a suitable buffered carrier. Expression of codon optimized hMERTK in transduced cells or retinas is assessed by retinal and visual function.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating a subject with an eye disease or disorder, the method comprising administering to the subject a nucleic acid comprising a nucleotide sequence at least 90% identical to any one of SEQ ID NOs: 3-6.

2. The method of claim 1, wherein the nucleic acid comprises a nucleotide sequence at least 90% identical to SEQ ID NO: 3.

3. The method of claim 1 or 2, wherein the nucleic acid comprises a nucleotide sequence at least 95% identical to SEQ ID NO: 3.

4. The method of any one of claims 1-3, wherein the nucleic acid comprises a nucleotide sequence of SEQ ID NO: 3.

5. The method of claim 1, wherein the nucleic acid comprises a nucleotide sequence at least 90% identical to SEQ ID NO: 4.

6. The method of claim 1 or 5, wherein the nucleic acid comprises a nucleotide sequence at least 95% identical to SEQ ID NO: 4.

7. The method of any one of claim 1 or 5-6, wherein the nucleic acid comprises a nucleotide sequence of SEQ ID NO: 4.

8. The method of claim 1, wherein the nucleic acid comprises a nucleotide sequence at least 90% identical to SEQ ID NO: 5.

9. The method of claim 1 or 8, wherein the nucleic acid comprises a nucleotide sequence at least 95% identical to SEQ ID NO: 5.

10. The method of any one of claim 1 or 8-9, wherein the nucleic acid comprises a nucleotide sequence of SEQ ID NO: 5.

11. The method of claim 1, wherein the nucleic acid comprises a nucleotide sequence at least 90% identical to SEQ ID NO: 6.

12. The method of claim 1 or 11, wherein the nucleic acid comprises a nucleotide sequence at least 95% identical to SEQ ID NO: 6.

13. The method of any one of claim 1 or 11-12, wherein the nucleic acid comprises a nucleotide sequence of SEQ ID NO: 6.

14. The method of any one of claims 1-13, wherein the nucleotide sequence is comprised in a vector.

15. The method of claim 14, wherein the vector is an adeno-associated viral (AAV) vector.

16. The method of claim 15, wherein the AAV vector is a recombinant AAV (rAAV) vector.

17. The method of claim 16, wherein the rAAV vector is selected from the group consisting of AAV2, AAV5, AAV8, AAV9, AAV2/5, AAV 2tYF, and AAV2.7m8.

18. The method of any one of claims 14-17, wherein the vector comprises an AAV capsid protein.

19. The method of claim 18, wherein the AAV capsid protein is selected from the group consisting of an AAV2 capsid protein, an AAV2tYF capsid protein, an AAV5 capsid protein, an AAV8 capsid protein, an AAV9 capsid protein, and a AAV2.7m8 capsid protein.

20. The method of any one of claims 14-19, wherein the vector further comprises: 1) a 5′ AAV ITR, and 2) a 3′ AAV ITR.

21. The method of any one of claims 14-20, wherein the vector comprises an AAV expression cassette.

22. The method of any one of claims 14-21, wherein the vector further comprises a rhodopsin (RHO) promoter.

23. The method of any one of claims 14-22, wherein the vector further comprises a vitelliform macular dystrophy (VMD2) promoter.

24. The method of any one of claims 14-23, wherein the vector further comprises a human rhodopsin kinase (hGRK1) promoter.

25. The method of any one of claims 14-24, wherein the vector further comprises a CBA promoter or a CASI promoter.

26. The method of any one of claims 1-25, wherein the disease or disorder comprises a MERTK-mediated disease or disorder.

27. The method of claim 26, wherein the MERTK-mediated disease comprises retinitis pigmentosa.

28. The method of any one of claims 1-27, wherein the disease or disorder comprises vision loss.

29. The method of any one of claims 1-28, wherein the administering occurs prior to onset of the disease or disorder.

30. The method of any one of claims 1-29, wherein the administering occurs after onset of the disease or disorder.

31. The method of any one of claims 1-30, wherein the administering occurs in at least one eye of the subject.

32. The method of any one of claims 1-31, wherein the administering is performed by subretinal injection, intravitreal injection, or suprachoroidal injection.

33. The method of any one of claims 1-32, wherein the administering is performed at an amount of at least 109 viral particles per mL.

34. The method of any one of claims 1-33, wherein the administering restores at least partial vision of the subject.

35. The method of any one of claims 1-34, wherein the administration improves vision loss by at least 10% when measured using a visual field test.

36. The method of any one of claims 1-35, wherein the subject is a mammal.

37. The method of any one of claims 1-36, wherein the subject is a human.

38. A functional MERTK nucleic acid sequence comprising a nucleic acid sequence at least 90%, 95%, or 99% identical to any one of SEQ ID NOs: 3-6.

39. A composition comprising the functional MERTK nucleic acid sequence of claim 38.

40. The composition of claim 39, further comprising a promoter which expresses a product of the functional MERTK nucleic acid sequence in a plurality of photoreceptor cells or retinal pigment epithelium cells.

41. The composition of any one of claim 39 or 40, further comprising a pharmaceutically acceptable carrier.

42. The composition of any one of claims 39-41, wherein the nucleic acid sequence comprises a nucleotide sequence at least 90% identical to SEQ ID NO: 3.

43. The composition of any one of claims 39-42, wherein the nucleic acid sequence comprises a nucleotide sequence at least 95% identical to SEQ ID NO: 3.

44. The composition of any one of claims 39-43, wherein the nucleic acid sequence comprises a nucleotide sequence of SEQ ID NO: 3.

45. The composition of any one of claims 39-41, wherein the nucleic acid sequence comprises a nucleotide sequence at least 90% identical to SEQ ID NO: 4.

46. The composition of any one of claim 39-41 or 45, wherein the nucleic acid sequence comprises a nucleotide sequence at least 95% identical to SEQ ID NO: 4.

47. The composition of any one of claim 39-41 or 45-46, wherein the nucleic acid sequence comprises a nucleotide sequence of SEQ ID NO: 4.

48. The composition of any one of claims 39-41, wherein the nucleic acid sequence comprises a nucleotide sequence at least 90% identical to SEQ ID NO: 5.

49. The composition of any one of claim 39-41 or 48, wherein the nucleic acid sequence comprises a nucleotide sequence at least 95% identical to SEQ ID NO: 5.

50. The composition of any one of claim 39-41 or 48-49, wherein the nucleic acid sequence comprises a nucleotide sequence of SEQ ID NO: 5.

51. The composition of any one of claims 39-41, wherein the nucleic acid sequence comprises a nucleotide sequence at least 90% identical to SEQ ID NO: 6.

52. The composition of any one of claims 39-41 or 51, wherein the nucleic acid sequence comprises a nucleotide sequence at least 95% identical to SEQ ID NO: 6.

53. The composition of any one of claims 39-41 or 51-52, wherein the nucleic acid sequence comprises a nucleotide sequence of SEQ ID NO: 6.

54. The composition of any one of claims 39-53, wherein the nucleic acid sequence is comprised in a vector.

55. The composition of claim 54, wherein the vector is an adeno-associated viral (AAV) vector.

56. The composition of claim 55, wherein the AAV vector is a recombinant AAV (rAAV) vector.

57. The composition of claim 56, wherein the rAAV vector is selected from the group consisting of AAV2, AAV5, AAV8, AAV9, AAV2/5, AAV 2tYF, and AAV2.7m8.

58. The composition of any one of claims 54-57, wherein the vector comprises an AAV capsid protein.

59. The composition of claim 58, wherein the AAV capsid protein is selected from the group consisting of an AAV2 capsid protein, an AAV2tYF capsid protein, an AAV5 capsid protein, an AAV8 capsid protein, an AAV9 capsid protein, and a AAV2.7m8 capsid protein.

60. The composition of any one of claims 54-59, wherein the vector further comprises: 1) a 5′ AAV ITR, and 2) a 3′ AAV ITR.

61. The composition of any one of claims 54-60, wherein the vector comprises an AAV expression cassette.

62. The composition of any one of claims 54-61, wherein the vector further comprises a rhodopsin (RHO) promoter.

63. The composition of any one of claims 54-62, wherein the vector further comprises a vitelliform macular dystrophy (VMD2) promoter.

64. The composition of any one of claims 54-63, wherein the vector further comprises a human rhodopsin kinase (hGRK1) promoter.

65. The composition of any one of claims 54-64, wherein the vector further comprises a CBA or a CASI promoter.

66. The composition of any one of claims 42-65, wherein the composition is for use to treat a disease or disorder.

67. The composition of claim 66, wherein the disease or disorder comprises a MERTK-mediated disease or disorder.

68. The composition of claim 67, wherein the MERTK-mediated disease comprises retinitis pigmentosa.

69. The composition of any one of claims 66-68, wherein the disease or disorder comprises vision loss.

70. The composition of any one of claims 66-69, wherein the composition is administered to a subject prior to onset of the disease or disorder.

71. The composition of any one of claims 66-70, wherein the composition is administered to a subject after onset of the disease or disorder.

72. The composition of any one of claims 39-71, wherein the composition is administered to a subject in at least one eye.

73. The composition of any one of claims 39-72, wherein the composition is administered to a subject by subretinal injection, intravitreal injection, or suprachoroidal injection.

74. The composition of any one of claims 39-73, wherein the composition comprises at least about 10⁹ viral particles per mL.

75. The composition of any one of claims 39-74, wherein the composition, when administered to a subject, restores at least partial vision to the subject.

76. The composition of any one of claims 39-75, wherein the composition, when administered to a subject, improves vision loss of the subject by at least 10% when measured using a visual field test.

77. Use of the functional MERTK nucleic acid sequence of claim 38 or the composition of any one of claims 39-76 for the manufacture of a medicament.

78. A kit comprising the functional MERTK nucleic acid sequence of claim 38 or the composition of any one of claims 39-76 and optionally instructions for use.

79. The method of any one of claims 1-37, wherein the administering increases internalization of photoreceptor outer segments (POS) by retinal pigment epithelium (RPE) when measured using a phagocytosis assay.

80. The method of claim 79, wherein the administering increases internalization of photoreceptor outer segments (POS) by retinal pigment epithelium (RPE) by at least 50% when measured using a phagocytosis assay.

81. The composition of any one of claims 39-76, wherein the composition, when administered to a subject, increases internalization of photoreceptor outer segments (POS) by retinal pigment epithelium (RPE) when measured using a phagocytosis assay.

82. The composition of claim 81, wherein the composition, when administered to a subject, increases internalization of photoreceptor outer segments (POS) by retinal pigment epithelium (RPE) by at least 50% when measured using a phagocytosis assay.


SEQUENCE LISTING (16 sequences)

SEQ ID NO: 1 - DNA, 3000 bp, Homo sapiens
  source 1..3000: mol_type = genomic DNA
atggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcagtgtacctaatatataccaggacaccacaatttcttggtggaaagatgggaaggaattgcttggggcacatcatgcaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatccccctccgtgctaactgttccaggcctgacggagatggcggtcttcagttgtgaggcccacaatgacaaagggctgaccgtgtccaagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgagtaatggctcagtcatgatttttaacacctctgccttaccacatctgtaccaaatcaagcagctgcaagccctggctaattacagcattggtgtttcctgcatgaatgaaataggctggtctgcagtgagcccttggattctagccagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtga

SEQ ID NO: 2 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 3 - DNA, 3000 bp, synthetic construct
  source 1..3000: mol_type = other DNA
atgggtcccgctcctctgcctctgctgctgggcctgttcctccctgccctgtggcggcgggccatcaccgaggccagagaagaggccaagccctaccctctgttccccggccctttccccggatccctgcaaacagaccacacccctctgctcagcctgcctcatgcctccggctatcagcctgccctgatgttcagccctactcagcctggccggcctcacaccggcaatgtggccatccctcaggtgacatccgtggaaagcaaacccctgcctcctcttgcctttaagcacacagtgggccacattatcctgagcgagcacaagggcgtgaagttcaactgcagtatatctgtgccgaacatctatcaagacacaaccatcagctggtggaaggacggaaaagaactgctgggagcccaccacgccatcacccagttctaccctgatgatgaagtgacagccatcatcgccagcttcagcatcaccagcgttcaaagaagcgacaacggcagctacatctgcaagatgaagatcaataacgaagagatcgtgagcgatcctatctacattgaggtccagggactgccccacttcaccaagcagccagaaagcatgaacgttacaagaaatactgccttcaacctgacctgccaggctgtgggcccacctgagccagtgaatatcttttgggtgcaaaattcctctagagtaaatgagcagcctgaaaagtctcctagcgtgctgaccgtgcctggcctgaccgagatggccgtcttcagctgcgaggctcacaacgataagggactgacagtgagcaagggcgttcaaatcaacatcaaggccatccccagcccaccaaccgaagtgtccatcagaaatagcaccgcccatagcatcctgattagctgggtgcctggttttgacggctactcccccttccggaactgtagcatccaggtgaaggaagccgatccgctgtcaaatggatctgtgatgatcttcaacaccagcgccctgcctcatctgtaccagatcaagcagctgcaagctctggccaactacagcatcggcgtgtcctgcatgaacgagatcggatggtccgctgtgtccccatggattcttgccagcaccaccgagggcgccccttctgttgccccactgaacgtgaccgtatttctgaacgagagcagcgacaacgtggacatcagatggatgaagccacctaccaagcagcaggatggcgagctggtgggctacaggatctcccacgtgtggcagagcgccggcatcagcaaggagctgctggaagaggtcggccagaatggcagcagagcccggatcagcgtccaggttcacaacgcaacctgtacagtcagaatcgccgccgtgacaagaggcggcgtgggccccttcagcgatcctgttaaaatcttcatccccgcccacggatgggtcgactacgcccctagcagcacccctgctcccggcaacgccgaccctgtgctgatcatcttcggatgcttctgcggctttatcctgatcggcctgatcctgtacatctccctggccatccgcaagcgggtgcaggagacaaagttcggcaacgccttcacagaagaggattccgaactggtggtgaactacatcgctaaaaagagcttctgcagaagagccatcgagctgacactgcacagcctgggagtgtctgaggaactgcagaacaagctggaagatgtggtcatcgacagaaacctgctcatactgggcaaaattctgggcgaaggagagttcggcagcgtgatggaaggcaacctgaagcaggaggacgggaccagcctgaaggtggctgtgaaaaccatgaagctggacaacagctcccagagggagatcgaggaattcctgagtgaagccgcctgcatgaaagacttttctcaccccaacgtgattagactgctgggcgtgtgtatcgaaatgagctctcagggcatccccaagcccatggtgatcctgcccttcatgaagtacggcgatctgcacacctacctgctgtattctagactggagacaggccctaagcacatccctctgcagaccctgctgaaattcatggtggatatcgccctgggcatggaatacctgagcaacagaaacttcctgcacagagaccttgctgcccggaactgcatgctgcgggacgacatgacggtgtgcgtggccgacttcggactgagcaagaaaatctacagcggcgactactacagacagggcagaatcgctaagatgcctgtgaagtggatcgccatcgagtctctggccgatagagtgtacacctcgaagagcgatgtgtgggcctttggagttacaatgtgggagatcgccaccagaggcatgaccccttatcctggcgtgcagaaccacgagatgtacgactacctgctgcacgggcaccgactgaaacagcccgaggactgcctggacgagctgtacgagatcatgtacagctgttggcggaccgaccctctggatcgccccacatttagcgtgctgcggctgcagctggaaaagctgctcgagagtctgcccgacgtgcggaaccaggccgatgtgatttacgtgaacacccagctgctggaaagctccgagggcctggcccaggggagcaccctggctcctctggacctgaatatcgacccagacagcattatcgcctcttgtaccccgagagccgctatcagcgtggtgaccgccgaggtgcacgacagcaagccccacgagggcagatacatcctgaacggaggctctgaggagtgggaagatctcacaagcgctccttctgccgccgtgaccgccgagaagaactccgtgctacctggcgagagacttgtgcggaacggcgtcagctggagccacagcagcatgctgcctctgggcagcagtcttcctgatgaactgctcttcgccgacgacagcagcgagggttcagaggtgctgatgtga

SEQ ID NO: 4 - DNA, 3000 bp, synthetic construct
  source 1..3000: mol_type = other DNA
atgggaccagctcctctgcctctgctgctgggactgtttctgccagctctctggaggagggccattacagaggccagagaggaggccaagccttaccctctgtttccaggccctttccccggatctctgcagaccgatcacacccctctgctgtctctgcctcacgctagcggataccagccagccctgatgtttagccctacccagccaggcagacctcacacaggaaacgtggccatccctcaggtcacaagcgtggagagcaagcctctgcctcctctggcctttaagcacaccgtgggacacatcatcctgagcgagcacaagggcgtgaagttcaattgcagcatcagcgtgcccaacatctaccaggacaccaccatcagttggtggaaggacggcaaggaactgctgggagctcatcacgccatcacccagttctaccccgacgacgaagtgaccgccatcatcgccagcttcagcatcaccagcgtgcagaggagcgacaacggcagctacatctgcaagatgaagatcaacaacgaggagatcgtgtccgaccccatctacatcgaggtgcagggactgcctcacttcaccaagcagccagagagcatgaacgtgacccggaacaccgccttcaacctgacttgccaggcagtgggacctccagagcccgtgaacatcttctgggtgcagaacagcagccgcgtgaacgaacagccagagaagagccctagcgtgctgacagtgccaggactgacagagatggccgtgttctcttgcgaggcccacaacgacaagggactgaccgtgtccaagggcgtgcagatcaacatcaaggccatccctagccctcctacagaggtgtccatcaggaacagcaccgcccacagcatcctgatctcttgggtgccaggcttcgacggatacagccccttccggaattgcagcatccaggtgaaggaggccgatcctctgagcaacggaagcgtgatgatcttcaacaccagcgccctgccccacctgtaccagatcaagcagctgcaggccctggctaactacagcatcggcgtgtcttgcatgaacgagatcggttggagcgccgtgagcccttggattctggccagcacaaccgagggagctccatcagtggctccactgaacgtgaccgtgttcctgaacgagagcagcgacaacgtggacatccgctggatgaagcctcccacaaagcagcaggacggagaactcgtgggctacagaatcagccacgtctggcagagtgccggaatcagcaaggagctgctggaggaagtgggccagaacggctctagagccaggatcagcgtgcaggtgcacaacgccacttgcaccgtgagaatcgccgcagtgacaagaggaggagtgggacccttcagcgaccccgtgaagatcttcatcccagctcacggttgggtggattacgctcctagcagcacaccagctccaggaaacgccgatcccgtgctgatcatcttcggctgcttctgcggcttcatcctgatcggcctgatcctgtacatcagcctggccatccggaagagagtgcaggagaccaagttcggcaacgccttcaccgaggaggacagcgagctggtcgtgaactacatcgccaagaagagcttttgccggagggccatcgagctgacactgcactctctgggagtgtccgaggagctgcagaacaagctggaggacgtcgtgatcgaccggaacctgctgattctgggcaagatcctgggcgagggagagtttggcagcgtgatggagggcaacctgaagcaggaggacggaacaagcctgaaggtggccgtgaagaccatgaagctggacaacagcagccagcgggagatcgaagagttcctgagcgaggccgcttgcatgaaggacttcagccaccccaacgtgatcaggctgctcggcgtctgtatcgagatgagcagccagggaatccccaagcccatggtcatcctgcccttcatgaagtacggcgacctgcacacctacctgctgtacagcaggctggagaccggacctaagcacatccctctgcagaccctgctgaagttcatggtggacatcgccctgggcatggagtacctgagcaaccggaacttcctgcacagagacctggccgctcggaattgcatgctgagagacgacatgaccgtctgcgtggcagacttcggcctgagcaagaagatctacagcggcgactactacagacagggcagaatcgccaagatgcccgtcaagtggatcgccatcgagagcctggccgatagggtgtacaccagcaagagcgacgtctgggctttcggagtgaccatgtgggagatcgccaccagaggcatgacaccttacccaggcgtgcagaaccacgagatgtacgactacctgctgcacggccacagactgaagcagccagaggattgcctggacgagctgtacgagatcatgtactcttgttggcggaccgaccctctggacagacctaccttcagcgtgctgaggctgcagctggaaaagctgctggagagcctgccagacgtcagaaatcaggccgacgtgatctacgtgaacacccagctgctggagagcagcgaaggactggctcagggctctacactggcccctctggatctgaacatcgaccccgatagcatcatcgcctcttgcacccctagagccgctatcagcgtggtgacagccgaagtgcacgacagcaagcctcacgagggacgctacatcctgaacggaggaagcgaggagtgggaggatctgacaagcgctccttctgctgccgtgacagccgaaaagaacagcgtgctgccaggagagaggctcgtgagaaacggcgtgtcttggagccacagcagcatgctgcctctgggaagctctctgccagacgagctgctctttgccgacgatagcagcgagggaagcgaggtgctgatgtag

SEQ ID NO: 5 - DNA, 3000 bp, synthetic construct
  source 1..3000: mol_type = other DNA
atgggacctgctcctctgcctctgctgctgggactgtttctgcctgctctttggcggagagccatcaccgaggccagagaggaagccaagccttatcctctgttccccggaccttttccaggcagcctgcagaccgatcacacccctttgctgtctctgcctcacgcctctggctatcagcccgctctgatgttcagccccacacagccaggcagacctcacacaggcaatgtggccattcctcaagtgaccagcgtggaaagcaagcccttgcctcctctggccttcaagcacacagtgggccacatcatcctgagcgagcacaagggcgtgaagttcaactgcagcatcagcgtgcccaacatctaccaggacaccaccatcagctggtggaaggacggcaaagaactgctgggagcccaccacgccatcacacagttctaccccgacgatgaagtgaccgccatcattgccagcttcagcatcaccagcgtgcagagaagcgacaacggcagctacatctgcaagatgaagatcaacaacgaggaaatcgtcagcgaccccatctacatcgaggtgcagggcctgcctcacttcaccaagcagcccgagagcatgaacgtgaccagaaacaccgccttcaacctgacctgtcaggccgtgggacctcctgagcctgtgaacatcttctgggtgcagaacagctccagagtgaacgagcagcctgagaagtcccctagcgtgctgacagtgcctggactgacagagatggccgtgttttcttgcgaggcccacaacgacaagggcctgaccgtgtctaagggcgtgcagatcaatatcaaggccattccatctccacctaccgaggtgtccatccggaatagcacagcccactccatcctgatctcttgggtgcccggcttcgatggctacagccccttcagaaactgctccatccaagtgaaagaggccgatcctctgagcaacggctccgtgatgatcttcaacacaagcgccctgccacacctgtaccagatcaaacagctgcaggctctggccaactactccatcggcgtgtcctgcatgaacgagatcggctggagtgccgtgtctccttggatcctggccagcacaactgaaggcgctccatctgtggcccctctgaatgtgaccgtgttcctgaacgagagcagcgacaatgtggacatccggtggatgaagccacctacaaagcagcaggacggcgaactcgtgggctacagaatctctcacgtgtggcagtctgccggcatctccaaagaactcctcgaagaagtgggccagaacggcagcagagccaggatctctgtgcaggtccacaacgccacatgcacagtgcggattgccgctgtgacaagaggcggagtgggcccttttagcgaccccgtgaagatctttatccccgctcacggctgggtcgactacgccccatcttctacaccagctccaggcaacgctgaccccgtgctgatcatcttcggctgcttttgcggctttatcctgatcggcctgatcctgtacatcagcctggccatcagaaagcgggtgcaagagacaaagttcggcaacgccttcaccgaagaggacagcgagctggtggtcaactatatcgccaagaagtccttctgcagacgggccatcgagctgacactgcacagtctgggagtgtccgaggaactgcagaacaagctggaagatgtggtcatcgaccggaacctgctgatcctgggcaagattctcggcgagggcgagtttggctctgtgatggaaggcaacctgaagcaagaggacggcacctctctgaaggtggccgtgaaaaccatgaagctggacaacagcagccagcgcgagatcgaagagtttctgtctgaggccgcctgtatgaaggatttctctcaccccaacgtgatccggctgctgggcgtgtgtatcgagatgtctagccagggcatccccaagcctatggtcatcctgcctttcatgaagtacggcgatctgcacacctacctgctgtactccagactggaaacaggccccaagcacatccctctgcagaccctgctgaagttcatggtggatatcgccctcggcatggaatacctgagcaaccggaacttcctgcaccgcgatctggccgccagaaattgcatgctgagggacgacatgaccgtgtgcgtggccgattttggcctgagcaagaagatctacagcggcgactactaccggcagggcagaattgccaagatgcccgtgaagtggatcgccatcgagagcctggccgacagagtgtacaccagcaagtctgacgtgtgggccttcggcgtgaccatgtgggagattgccaccagaggcatgaccccttatcctggcgtccagaaccacgagatgtacgattacctgctgcacggccacagactgaagcagccagaggattgcctggacgagctgtacgagatcatgtactcttgctggcggaccgatccactggacagacctacattctccgtgctgcggctgcagctggaaaaactgctggaaagcctgcctgacgtgcggaaccaggccgatgtgatctacgtgaacacccagctgctggaatccagcgaaggactggcccagggatctacactggctcctctggacctgaacatcgaccccgacagcattatcgccagctgcacaccaagagccgccatcagcgttgtgacagccgaggtgcacgatagcaagcctcacgaaggccggtacatcctgaatggcggaagcgaggaatgggaagatctgaccagcgctccttctgccgccgtgaccgccgagaaaaattctgtgctgcctggcgagcggctcgtgcgaaacggtgtctcttggagccacagctccatgctgcctctgggaagctctctgcctgatgagctgctgttcgccgacgattctagcgagggatccgaggtgctgatgtga

SEQ ID NO: 6 - DNA, 3000 bp, synthetic construct
  source 1..3000: mol_type = other DNA
atgggaccagcgccattgcctttgttgctcgggttatttctgccagccctgtggcggcgtgccattacagaagctcgagaggaagctaaaccctatccattgtttccaggtcccttccctggctcattgcagacagatcatacgcctctcttgtcattaccacatgctagcggctatcaaccagctctcatgttctctcctacacaacccggccggccccacaccggtaatgtggcgatcccacaagtcactagtgtagagagcaaaccactgccccctctggcttttaagcatactgtcgggcatatcatcctgagtgagcacaagggagttaagttcaactgtagtatttccgtccccaacatttatcaagatacgaccatatcctggtggaaggacggtaaagagctgttgggcgctcaccacgccatcacccaattctaccctgacgacgaggtgactgccatcatagcatccttttcaatcacgagcgtacaaaggtccgataacggctcttacatatgcaaaatgaagattaataacgaggaaattgttagtgacccgatttatattgaggtgcagggcctgccccatttcacaaaacaaccggaatccatgaacgtaactcgaaataccgcttttaatttgacttgccaagccgttggacccccagaaccggtaaatatcttttgggtgcagaatagctcacgagtaaatgagcaacccgagaagtctccgtctgttttaacagtccctgggcttacagaaatggcagtgttttcttgcgaagcgcataacgataagggtttgacagtcagcaaaggcgtacaaattaatataaaggccatcccatcaccacccacagaggtttcaatacgcaattcaacagcccattcaatcctcatttcttgggtgcccggattcgacgggtatagcccttttagaaactgttcaatccaagtgaaagaggccgaccctttatccaacgggagcgtgatgatcttcaatacttcagcactccctcacctttatcagattaaacaacttcaggcgttggccaactattccatcggggtgagttgtatgaacgagattgggtggagcgctgtatctccatggatccttgctagtactactgagggtgcacccagcgtggctcccttgaacgtgaccgtcttcctcaacgagtcatccgacaacgtcgatattcgatggatgaaaccacccaccaaacaacaagacggtgagttagtcggatatagaattagtcatgtttggcaaagcgctggtatcagtaaggaattgctggaagaggtcggacaaaacggaagtcgcgccaggataagtgtgcaggtgcataacgccacttgtactgtaagaatcgctgctgtgacacgaggcggcgtcggcccgtttagcgaccctgtaaagattttcattccagcgcatgggtgggtggactacgctcctagctctacccctgcccccgggaatgctgacccggtccttattattttcggatgtttctgcgggttcatcctgataggtctcatattgtatatttctctggcaattcgtaagcgcgtgcaagaaaccaaattcggtaacgcctttactgaagaagacagcgagctggtcgtcaactacatcgctaagaagagcttttgcaggagagctatagagctgacacttcactcactgggtgttagcgaagagctgcagaacaagctggaagacgtggtcatcgatcgaaacctgctcatactgggcaagatcttgggcgaaggggaattcggttccgtgatggaaggcaacctgaaacaagaggacggcactagcctcaaggttgccgtcaaaacaatgaaactcgataatagtagtcaaagagaaattgaagaattcctgtctgaagccgcctgtatgaaggatttctcccatccgaacgtgatcaggctcttgggcgtctgcattgagatgtctagccaggggattcctaaacctatggttatcctgccatttatgaagtatggtgatttgcacacatatctcttatacagccgcctcgaaaccggccccaaacacatcccattgcaaactcttctgaaatttatggtcgacatcgctttagggatggaatacttgagtaatcgaaacttcctgcaccgggacctggcggccagaaattgtatgctgagagacgatatgaccgtgtgcgtggccgattttggattgtcaaagaaaatctatagcggtgactactataggcaggggagaatcgccaaaatgcccgtgaagtggattgcaatcgagagcctggcggatagagtgtatacttcaaagtccgacgtctgggccttcggtgtcactatgtgggagatcgcgactcgagggatgaccccgtaccccggagttcaaaatcacgaaatgtacgattacttacttcacgggcatcgcctcaaacaaccagaggattgtcttgacgagctctacgagatcatgtatagttgttggcgcacagaccctctggatcggcctacattctctgtgctccgcttacaattggagaagttgctcgagtctctcccagatgtgagaaatcaggctgatgtgatctatgtgaacacccaactgcttgaatccagcgaaggacttgcgcaagggtctacactcgcccccttagatctcaatattgatccagattcaatcatcgcgagctgtaccccaagagcggcaattagcgttgtgaccgccgaggtgcacgattccaagccccacgaggggcgctatattttaaacggcggatctgaagagtgggaagacttaacaagtgcacctagcgcagctgtaactgccgagaagaattctgtacttcccggtgaacgcctggtgcggaacggagtgagttggtcacacagctccatgctcccactgggttcaagtcttccggacgagctcctgttcgcagatgattcttccgagggatccgaggtgcttatgtag

SEQ ID NO: 7 - DNA, 7017 bp, synthetic construct
  source 1..7017: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtttaaacctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcgcggccgcggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgcgatcgcaggtaagtttagtctttttgtcttttatttcaggtcccggatccggtggtggtgcaaatcaaagaactgctcctcagtggatgttgcctttacttctaggcctgcaggccgccaccatgggacctgctcctctgcctctgctgctgggactgtttctgcctgctctttggcggagagccatcaccgaggccagagaggaagccaagccttatcctctgttccccggaccttttccaggcagcctgcagaccgatcacacccctttgctgtctctgcctcacgcctctggctatcagcccgctctgatgttcagccccacacagccaggcagacctcacacaggcaatgtggccattcctcaagtgaccagcgtggaaagcaagcccttgcctcctctggccttcaagcacacagtgggccacatcatcctgagcgagcacaagggcgtgaagttcaactgcagcatcagcgtgcccaacatctaccaggacaccaccatcagctggtggaaggacggcaaagaactgctgggagcccaccacgccatcacacagttctaccccgacgatgaagtgaccgccatcattgccagcttcagcatcaccagcgtgcagagaagcgacaacggcagctacatctgcaagatgaagatcaacaacgaggaaatcgtcagcgaccccatctacatcgaggtgcagggcctgcctcacttcaccaagcagcccgagagcatgaacgtgaccagaaacaccgccttcaacctgacctgtcaggccgtgggacctcctgagcctgtgaacatcttctgggtgcagaacagctccagagtgaacgagcagcctgagaagtcccctagcgtgctgacagtgcctggactgacagagatggccgtgttttcttgcgaggcccacaacgacaagggcctgaccgtgtctaagggcgtgcagatcaatatcaaggccattccatctccacctaccgaggtgtccatccggaatagcacagcccactccatcctgatctcttgggtgcccggcttcgatggctacagccccttcagaaactgctccatccaagtgaaagaggccgatcctctgagcaacggctccgtgatgatcttcaacacaagcgccctgccacacctgtaccagatcaaacagctgcaggctctggccaactactccatcggcgtgtcctgcatgaacgagatcggctggagtgccgtgtctccttggatcctggccagcacaactgaaggcgctccatctgtggcccctctgaatgtgaccgtgttcctgaacgagagcagcgacaatgtggacatccggtggatgaagccacctacaaagcagcaggacggcgaactcgtgggctacagaatctctcacgtgtggcagtctgccggcatctccaaagaactcctcgaagaagtgggccagaacggcagcagagccaggatctctgtgcaggtccacaacgccacatgcacagtgcggattgccgctgtgacaagaggcggagtgggcccttttagcgaccccgtgaagatctttatccccgctcacggctgggtcgactacgccccatcttctacaccagctccaggcaacgctgaccccgtgctgatcatcttcggctgcttttgcggctttatcctgatcggcctgatcctgtacatcagcctggccatcagaaagcgggtgcaagagacaaagttcggcaacgccttcaccgaagaggacagcgagctggtggtcaactatatcgccaagaagtccttctgcagacgggccatcgagctgacactgcacagtctgggagtgtccgaggaactgcagaacaagctggaagatgtggtcatcgaccggaacctgctgatcctgggcaagattctcggcgagggcgagtttggctctgtgatggaaggcaacctgaagcaagaggacggcacctctctgaaggtggccgtgaaaaccatgaagctggacaacagcagccagcgcgagatcgaagagtttctgtctgaggccgcctgtatgaaggatttctctcaccccaacgtgatccggctgctgggcgtgtgtatcgagatgtctagccagggcatccccaagcctatggtcatcctgcctttcatgaagtacggcgatctgcacacctacctgctgtactccagactggaaacaggccccaagcacatccctctgcagaccctgctgaagttcatggtggatatcgccctcggcatggaatacctgagcaaccggaacttcctgcaccgcgatctggccgccagaaattgcatgctgagggacgacatgaccgtgtgcgtggccgattttggcctgagcaagaagatctacagcggcgactactaccggcagggcagaattgccaagatgcccgtgaagtggatcgccatcgagagcctggccgacagagtgtacaccagcaagtctgacgtgtgggccttcggcgtgaccatgtgggagattgccaccagaggcatgaccccttatcctggcgtccagaaccacgagatgtacgattacctgctgcacggccacagactgaagcagccagaggattgcctggacgagctgtacgagatcatgtactcttgctggcggaccgatccactggacagacctacattctccgtgctgcggctgcagctggaaaaactgctggaaagcctgcctgacgtgcggaaccaggccgatgtgatctacgtgaacacccagctgctggaatccagcgaaggactggcccagggatctacactggctcctctggacctgaacatcgaccccgacagcattatcgccagctgcacaccaagagccgccatcagcgttgtgacagccgaggtgcacgatagcaagcctcacgaaggccggtacatcctgaatggcggaagcgaggaatgggaagatctgaccagcgctccttctgccgccgtgaccgccgagaaaaattctgtgctgcctggcgagcggctcgtgcgaaacggtgtctcttggagccacagctccatgctgcctctgggaagctctctgcctgatgagctgctgttcgccgacgattctagcgagggatccgaggtgctgatgtgattaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcgcgccttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcaggtttaaacatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 8 - DNA, 7017 bp, synthetic construct
  source 1..7017: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtttaaacctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcgcggccgcggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgcgatcgcaggtaagtttagtctttttgtcttttatttcaggtcccggatccggtggtggtgcaaatcaaagaactgctcctcagtggatgttgcctttacttctaggcctgcaggccgccaccatggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcagtgtacctaatatataccaggacaccacaatttcttggtggaaagatgggaaggaattgcttggggcacatcatgcaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatccccctccgtgctaactgttccaggcctgacggagatggcggtcttcagttgtgaggcccacaatgacaaagggctgaccgtgtccaagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgagtaatggctcagtcatgatttttaacacctctgccttaccacatctgtaccaaatcaagcagctgcaagccctggctaattacagcattggtgtttcctgcatgaatgaaataggctggtctgcagtgagcccttggattctagccagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtgattaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcgcgccttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcaggtttaaacatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 9 - DNA, 6981 bp, synthetic construct
  source 1..6981: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtttaaacctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcgcggccgccgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccgcgatcgcaggtaagtttagtctttttgtcttttatttcaggtcccggatccggtggtggtgcaaatcaaagaactgctcctcagtggatgttgcctttacttctaggcctgcaggccgccaccatgggacctgctcctctgcctctgctgctgggactgtttctgcctgctctttggcggagagccatcaccgaggccagagaggaagccaagccttatcctctgttccccggaccttttccaggcagcctgcagaccgatcacacccctttgctgtctctgcctcacgcctctggctatcagcccgctctgatgttcagccccacacagccaggcagacctcacacaggcaatgtggccattcctcaagtgaccagcgtggaaagcaagcccttgcctcctctggccttcaagcacacagtgggccacatcatcctgagcgagcacaagggcgtgaagttcaactgcagcatcagcgtgcccaacatctaccaggacaccaccatcagctggtggaaggacggcaaagaactgctgggagcccaccacgccatcacacagttctaccccgacgatgaagtgaccgccatcattgccagcttcagcatcaccagcgtgcagagaagcgacaacggcagctacatctgcaagatgaagatcaacaacgaggaaatcgtcagcgaccccatctacatcgaggtgcagggcctgcctcacttcaccaagcagcccgagagcatgaacgtgaccagaaacaccgccttcaacctgacctgtcaggccgtgggacctcctgagcctgtgaacatcttctgggtgcagaacagctccagagtgaacgagcagcctgagaagtcccctagcgtgctgacagtgcctggactgacagagatggccgtgttttcttgcgaggcccacaacgacaagggcctgaccgtgtctaagggcgtgcagatcaatatcaaggccattccatctccacctaccgaggtgtccatccggaatagcacagcccactccatcctgatctcttgggtgcccggcttcgatggctacagccccttcagaaactgctccatccaagtgaaagaggccgatcctctgagcaacggctccgtgatgatcttcaacacaagcgccctgccacacctgtaccagatcaaacagctgcaggctctggccaactactccatcggcgtgtcctgcatgaacgagatcggctggagtgccgtgtctccttggatcctggccagcacaactgaaggcgctccatctgtggcccctctgaatgtgaccgtgttcctgaacgagagcagcgacaatgtggacatccggtggatgaagccacctacaaagcagcaggacggcgaactcgtgggctacagaatctctcacgtgtggcagtctgccggcatctccaaagaactcctcgaagaagtgggccagaacggcagcagagccaggatctctgtgcaggtccacaacgccacatgcacagtgcggattgccgctgtgacaagaggcggagtgggcccttttagcgaccccgtgaagatctttatccccgctcacggctgggtcgactacgccccatcttctacaccagctccaggcaacgctgaccccgtgctgatcatcttcggctgcttttgcggctttatcctgatcggcctgatcctgtacatcagcctggccatcagaaagcgggtgcaagagacaaagttcggcaacgccttcaccgaagaggacagcgagctggtggtcaactatatcgccaagaagtccttctgcagacgggccatcgagctgacactgcacagtctgggagtgtccgaggaactgcagaacaagctggaagatgtggtcatcgaccggaacctgctgatcctgggcaagattctcggcgagggcgagtttggctctgtgatggaaggcaacctgaagcaagaggacggcacctctctgaaggtggccgtgaaaaccatgaagctggacaacagcagccagcgcgagatcgaagagtttctgtctgaggccgcctgtatgaaggatttctctcaccccaacgtgatccggctgctgggcgtgtgtatcgagatgtctagccagggcatccccaagcctatggtcatcctgcctttcatgaagtacggcgatctgcacacctacctgctgtactccagactggaaacaggccccaagcacatccctctgcagaccctgctgaagttcatggtggatatcgccctcggcatggaatacctgagcaaccggaacttcctgcaccgcgatctggccgccagaaattgcatgctgagggacgacatgaccgtgtgcgtggccgattttggcctgagcaagaagatctacagcggcgactactaccggcagggcagaattgccaagatgcccgtgaagtggatcgccatcgagagcctggccgacagagtgtacaccagcaagtctgacgtgtgggccttcggcgtgaccatgtgggagattgccaccagaggcatgaccccttatcctggcgtccagaaccacgagatgtacgattacctgctgcacggccacagactgaagcagccagaggattgcctggacgagctgtacgagatcatgtactcttgctggcggaccgatccactggacagacctacattctccgtgctgcggctgcagctggaaaaactgctggaaagcctgcctgacgtgcggaaccaggccgatgtgatctacgtgaacacccagctgctggaatccagcgaaggactggcccagggatctacactggctcctctggacctgaacatcgaccccgacagcattatcgccagctgcacaccaagagccgccatcagcgttgtgacagccgaggtgcacgatagcaagcctcacgaaggccggtacatcctgaatggcggaagcgaggaatgggaagatctgaccagcgctccttctgccgccgtgaccgccgagaaaaattctgtgctgcctggcgagcggctcgtgcgaaacggtgtctcttggagccacagctccatgctgcctctgggaagctctctgcctgatgagctgctgttcgccgacgattctagcgagggatccgaggtgctgatgtgattaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcgcgccttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcaggtttaaacatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 10 - DNA, 6981 bp, synthetic construct
  source 1..6981: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgtttaaacctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcgcggccgccgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccgcgatcgcaggtaagtttagtctttttgtcttttatttcaggtcccggatccggtggtggtgcaaatcaaagaactgctcctcagtggatgttgcctttacttctaggcctgcaggccgccaccatggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcagtgtacctaatatataccaggacaccacaatttcttggtggaaagatgggaaggaattgcttggggcacatcatgcaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatccccctccgtgctaactgttccaggcctgacggagatggcggtcttcagttgtgaggcccacaatgacaaagggctgaccgtgtccaagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgagtaatggctcagtcatgatttttaacacctctgccttaccacatctgtaccaaatcaagcagctgcaagccctggctaattacagcattggtgtttcctgcatgaatgaaataggctggtctgcagtgagcccttggattctagccagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtgattaattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcgcgccttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcaggtttaaacatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 11 - DNA, 24 bp, synthetic construct
  source 1..24: mol_type = other DNA
ccactggacttgaacatcgaccct

SEQ ID NO: 12 - DNA, 20 bp, synthetic construct
  source 1..20: mol_type = other DNA
ggagtgcaggaggcaattat

SEQ ID NO: 13 - DNA, 22 bp, synthetic construct
  source 1..22: mol_type = other DNA
tcaatacacagttgctggagag

SEQ ID NO: 14 - DNA, 24 bp, synthetic construct
  source 1..24: mol_type = other DNA
atcgagctgacactgcacagtctg

SEQ ID NO: 15 - DNA, 20 bp, synthetic construct
  source 1..20: mol_type = other DNA
gaccacatcttccagcttgt

SEQ ID NO: 16 - DNA, 22 bp, synthetic construct
  source 1..22: mol_type = other DNA
cgagctggtggtcaactatatc